(19) United States
(12) Patent Application Publication
(10) Pub. No.: US 2025/0092043 A1
(43) Pub. Date: Mar. 20, 2025

(54) Kinase Inhibitors and Degraders

(71) Applicant: Incyte Corporation, Wilmington, DE (US)

(72) Inventors: David M. Burns, Plymouth Meeting, PA (US); Yanran Ai, West Chester, PA (US); Kai Liu, Chadds Ford, PA (US); Cooper Ashley Taylor, Wilmington, DE (US); Eddy W. Yue, Landenberg, PA (US); Zhitong Zheng, Glen Mills, PA (US)

(21) Appl. No.: 18/827,205

(22) Filed: Sep. 6, 2024

Related U.S. Application Data
(60) Provisional application No. 63/537,327, filed on Sep. 8, 2023.

(51) Int. Cl. C07D 471/14; A61K 31/437; A61K 31/4545; A61K 31/496; A61K 31/5377

(52) U.S. Cl. CPC C07D 471/14; A61K 31/437; A61K 31/4545; A61K 31/496; A61K 31/5377

(57) ABSTRACT

The present application provides heterocyclic compounds that modulate the activity of JAK2, which are useful in the treatment of various diseases, including cancer.

### TECHNICAL FIELD

The present invention provides heterocyclic compounds that modulate the activity of Janus kinase(s) (JAK) through binding to the JH2 or pseudokinase domain and either inhibiting or degrading the JAK(s), which are useful, for example, in the treatment of diseases in which the JAK-STAT signaling pathway is dysregulated, including cancer, inflammatory, autoimmune, and immune related diseases.

### BACKGROUND

Janus kinase (JAK) 2 μlays pivotal roles in signaling by several cytokine receptors. The mutant JAK2 V617F is the most common molecular event associated with myeloproliferative neoplasms and has also been associated with autoimmune related diseases. Selective targeting of the JAK2 V617F mutant using a small molecule inhibitor or targeted protein degrader may be useful for treating various pathologies, while sparing essential JAK2 functions.

### SUMMARY

The present invention relates to, inter alia, compounds of Formula I.

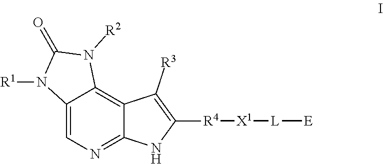

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides methods of treating a disease or a disorder associated with expression or activity of the V617F variant of JAK2 kinase in a patient by administering to a patient a therapeutically effective amount of a compound of Formula I, or a pharmaceutically acceptable salt thereof.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

Targeted protein degradation (TPD) can occur through various mechanisms including molecular glues, lysosome-targeted chimeras (LYTACs), and proteolysis targeting chimeras (PROTAC®s). PROTAC®s may also be referred to as specific and non-genetic inhibitor of apoptosis (IAP)-dependent protein erasers (SNIPERs) and chemical inducers of degradation (CIDEs). PROTAC®s are heterobifunctional molecules consisting of three components: protein of interest (POI) inhibitor, linker, and E3 ligase recruiter. The PROTAC® can form a ternary complex by binding to both the POI and an E3 ligase, such as cereblon (CRBN), inhibitor of apoptosis protein (IAP), Von Hippel-Lindau (VHL), mouse double minute 2 (MDM2), and the like. Upon ternary complex formation the POI is polyubiquitinated and marked for proteasome-mediated degradation utilizing the ubiquitin-proteasome system (UPS). Protein-protein interactions during the formation of the ternary complex can lead to enhanced selectivity of a PROTAC® vs. its parent compound due to cooperativity (see e.g., Crews, G. M. et al. Cell Chem. Biol. 2018, 25, 78; and Ciulli, A. et al. Nat. Chem. Biol. 2017, 13, 514). These differences in selectivity profiles may offer several advantages for PROTAC®s in comparison to conventional small molecule inhibitors. Because PROTAC®s degrade a POI, in contrast to conventional small molecules that inhibit the POI, some divergence may occur in pharmacological effects, safety profile, drug resistance, and dosing. PROTAC®s may be used to selectively degrade JAK for the treatment of JAK related pathologies, including: cancers, inflammatory, and immune related diseases. In particular, it may be advantageous to predominately degrade individual isoforms of JAK, such as JAK2, and/or mutated JAKs, such as V617F JAK2, or various combinations of isoforms and/or mutated JAKs (e.g. degradation of JAK2, JAK1/JAK2, V617F JAK2, or V617F JAK2/WT JAK2).

Accordingly, the present application provides a compound of Formula I:

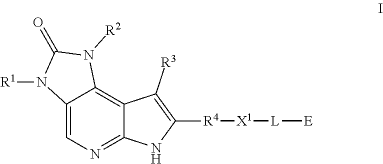

or a pharmaceutically acceptable salt thereof, wherein:
R¹ is selected from H, C₁₋₆ alkyl, C₂₋₆ alkenyl, and C₂₋₆ alkynyl, wherein the C₁₋₆ alkyl, C₂₋₆ alkenyl, and C₂₋₆ alkynyl of R¹ are each optionally substituted with 1, 2, or 3 independently selected R^{1A} substituents;
each R^{1A} is independently selected from halo, oxo, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}NR^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11};
each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{a11}, R^{b11}, R^{c11} and R^{d11} are each optionally substituted with 1, 2, 3, 4, 5, 6, 7, or 8 independently selected R^{M} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, and C₂₋₆ alkynyl;
R² is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{2A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}R^{c21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f21} and R^{g21} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h21} and Rⁱ²¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)R^{b22}, NR^{c22}S(O)NR^{c22}R^{d22}, NR^{c22}S(O)₂R^{b22}, NR^{c22}S(O)(═NR^{e22})R^{b22}, NR^{c22}S(O)₂NR^{c22}R^{d22} S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2B} are each optionally substituted with 1, 2, 3, 4, 5, 6, 7, or 8 independently selected R²¹ substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a22}, R^{c22} and R^{d22} are each optionally substituted with 1, 2, 3, 4, 5, 6, 7, or 8 independently selected R^{2C} substituents;
or, any R^{c22} and R^{d22} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2C} substituents;
each R^{b22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b22} are each optionally substituted with 1, 2, 3, 4, 5, 6, 7, or 8 independently selected R^{2C} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f22} and R^{g22} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h22} and Rⁱ²² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a23}, SR^{a23}, NHOR^{a23}, C(O)R^{b23}, C(O)NR^{e23}R^{d23}, C(o)NR^{c23}(OR^{a23}), C(O)OR^{a23}, OC(O)R^{b23}, OC(O)NR^{c23}R^{d23}, NR^{c23}R^{d23}, NR^{c23}NR^{c23}R^{d23}, NR^{c23}C(O)R^{b23}, NR^{c23}C(O)OR^{a23}, NR^{c23}C(O)NR^{c23}R^{d23}C(═NR^{e23})R^{b23}, C(═NR^{e23})NR^{c23}R^{d23}, NR^{c23}C(═NR^{e23})NR^{c23}R^{d23}, NR^{c23}C(═NR^{e23})R^{b23}, NR^{c23}S(O)R^{b23}, NR^{c23}S(O)NR^{c23}R^{d23}, NR^{e23}S(O)₂R^{b23}, NR^{c23}S(O)(═NR^{e23})R^{b23}, NR^{c23}S(O)₂NR^{c23}R^{d23}, S(O)R^{b23}, S(O)NR^{e23}R^{d23}, S(O)₂R^{b23}, S(O)₂NR^{c23}R^{d23}, OS(O)(═NR^{e23})R^{b23}, and OS(O)₂R^{b23}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{2C} are each optionally substituted with 1, 2, 3, or 4 independently selected R^{M} substituents;
each R^{a23}, R^{c23}, and R^{d23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a23}, R^{c23} and R^{d23} are each optionally substituted with 1, 2, 3, or 4 independently selected R^{M} substituents;
or, any R^{c23} and R^{d23} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b23} are each optionally substituted with 1, 2, 3, or 4 independently selected R^{M} substituents;
each R^{e23} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R³ is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)NR^{c3}R^{d3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, 4, 5, 6, 7, or 8 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a3}, R^{c3} and R^{d3} are each optionally substituted with 1, 2, 3, 4, 5, 6, 7, or 8 independently selected R^{3A} substituents;
or, any R^{c3} and R^{d3} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3A} substituents;
each R^{b3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b3} are each optionally substituted with 1, 2, 3, 4, 5, 6, 7, or 8 independently selected R^{3A} substituents;
each R^{c3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f3} and R^{g3} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h3} and Rⁱ³ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j3} and R^{k3} is independently selected from OH, C₁₋₆ alkoxy, and C₁₋₆ haloalkoxy;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a31}, SR^{a31}, NHOR^{a31}, C(O)R^{b31}, C(O)NR^{c31}R^{d31}, C(O)NR^{c31}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}C(O)NR^{c31}R^{d31}C(═NR^{e31})R^{b31}, C(═NR^{e31})NR^{c31}R^{d31}, NR^{c31}C(═NR^{e31})NR^{c31}R^{d31}, NR^{c31}C(═NR^{e31})R^{b31}, NR^{c31}S(O)R^{b31}, NR^{c31}S(O)NR^{c31}R^{d31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, SF₅, P(O)R^{f31}R^{g31}, OP(O)(OR^{h31})(ORⁱ³¹), P(O)(OR^{h31})(ORⁱ³), and BR^{j31}R^{k31}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{3A} are each optionally substituted with 1, 2, 3, 4, 5, 6, 7, or 8 independently selected R^{3B} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a31}, R^{c31} and R^{d31} are each optionally substituted with 1, 2, 3, 4, 5, 6, 7, or 8 independently selected R^{3B} substituents;
or, any R^{c31} and R^{d31} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3B} substituents;
each R^{b31} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b31} are each optionally substituted with 1, 2, 3, 4, 5, 6, 7, or 8 independently selected R^{3B} substituents;
each R^{c31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f31} and R^{g31} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h31} and Rⁱ³¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j31} and R^{k31} is independently selected from OH, C₁₋₆ alkoxy, and C₁₋₆ haloalkoxy;
or any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a32}, SR^{a32}, NHOR^{a32}, C(O)R^{b32}, C(O)NR^{c32}R^{d32}, C(O)NR^{c32}(OR^{a32}), C(O)OR^{a32}, OC(O)R^{b32}, OC(O)NR^{c32}R^{d32}, NR^{c32}R^{d32}, NR^{c32}NR^{c32}R^{d32}, NR^{c32}C(O)R^{b32}, NR^{c32}C(O)OR^{a32}, NR^{c32}C(O)NR^{c32}R^{d32}C(═NR^{e32})R^{b32}, C(═NR^{e32})NR^{c32}R^{d32}, NR^{c32}C(═NR^{e32})NR^{c32}R^{d32}, NR^{c32}C(═NR^{e32})R^{b32}, NR^{c32}S(O)R^{b32}, NR^{c32}S(O)NR^{c32}R^{d32}, NR^{c32}S(O)₂R^{b32}, NR^{c32}S(O)(═NR^{e32})R^{b32}, NR^{c32}S(O)₂NR^{c32}R^{d32} S(O)R^{b32}, S(O)NR^{c32}R^{d32}, S(O)₂R^{b32}, S(O)₂NR^{c32}R^{d32}, OS(O)(═NR^{e32})R^{b32}, OS(O)₂R^{b32}, SF₅, P(O)R^{f32}R^{g32}, OP(O)(OR^{h32})(ORⁱ³²), P(O)(OR^{h32})(ORⁱ³²), and BR^{j32}R^{k32}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{3B} are each optionally substituted with 1, 2, 3, 4, 5, 6, 7, or 8 independently selected R^{3C} substituents;
each R^{a32}, R^{c32}, and R^{d32} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a32}, R^{c32} and R^{d32} are each optionally substituted with 1, 2, 3, 4, 5, 6, 7, or 8 independently selected R^{3C} substituents;
or, any R^{c32} and R^{d32} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3C} substituents;
each R^{b32} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b32} are each optionally substituted with 1, 2, 3, 4, 5, 6, 7, or 8 independently selected R^{3C} substituents;
each R^{e32} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f32} and R^{g32} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h32} and Rⁱ³² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j32} and R^{k32} is independently selected from OH, C₁₋₆ alkoxy, and C₁₋₆ haloalkoxy;
or any R^{j32} and R^{k32} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a3}, SR^{a33}, NHOR^{a33}, C(O)R^{b33}, C(O)NR^{c33}R^{d33}, C(O)NR^{c33}(OR^{a33}), C(O)OR^{a33}, OC(O)R^{b33}, OC(O)NR^{c33}R^{d33}, NR^{c33}R^{d33}, NR^{c33}NR^{c33}R^{d33}, NR^{c33}C(O)R^{b33}, NR^{c33}C(O)OR^{a33}, NR^{c33}C(O)NR^{c33}R^{d33}, C(═NR^{e33})R^{b33}, C(═NR^{e33})NR^{c33}R^{d33}, NR^{c33}C(═NR^{e33})NR^{c33}R^{d33}, NR^{c33}C(═NR^{e33})R^{b33}, NR^{c33}S(O)R^{b33}, NR^{c33}S(O)NR^{c33}R^{d33}, NR^{c33}S(O)₂R^{b33}, NR^{c33}S(O)(═NR^{e33})R^{b33}, NR^{c33}S(O)₂NR^{c33}R^{d33}, S(O)R^{b33}, S(O)NR^{c33}R^{d33}, S(O)₂R^{b33}, S(O)₂NR^{c33}R^{d33}, OS(O)(═NR^{e33})R^{b33}, and OS(O)₂R^{b33}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{3C} are each optionally substituted with 1, 2, 3, or 4 independently selected R^{M} substituents;
each R^{a33}, R^{c33}, and R^{d33} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a33}, R^{c33} and R^{d33} are each optionally substituted with 1, 2, 3, or 4 independently selected R^{M} substituents;
or, any R^{c33} and R^{d33} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b33} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b33} are each optionally substituted with 1, 2, 3, or 4 independently selected R^{M} substituents;
each R^{c33} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from C₁₋₆ alkylene, C₁₋₆ haloalkylene, C₂₋₆ alkenylene, C₂₋₆ alkynylene, C₆₋₁₀ arylene, C₃₋₁₀ cycloalkylene, 5-10 membered heteroarylene, 4-10 membered heterocycloalkylene, —C₆₋₁₀ arylene-C₁₋₆ alkylene-, —C₃₋₁₀ cycloalkylene-C₁₋₆ alkylene-, -(5-10 membered heteroarylene)-C₁₋₆ alkylene-, and -(4-10 membered heterocycloalkylene)-C₁₋₆ alkylene-, wherein the C₁₋₆ alkylene, C₂₋₆ alkenylene, C₂₋₆ alkynylene, C₆₋₁₀ arylene, C₃₋₁₀ cycloalkylene, 5-10 membered heteroarylene, 4-10 membered heterocycloalkylene, —C₆₋₁₀ aryl-C₁₋₆ alkylene-, —C₃₋₁₀ cycloalkyl-C₁₋₆ alkylene-, -(5-10 membered heteroaryl)-C₁₋₆ alkylene-, and -(4-10 membered heterocycloalkyl)-C₁₋₆ alkylene- of R⁴ are each optionally substituted with 1, 2, 3, 4, 5, 6, 7, or 8 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, SF₅, P(O)R^{f41}R^{g41}, OP(O)(OR^{h41})(ORⁱ⁴¹), P(O)(OR^{h41})(ORⁱ⁴¹), and BR^{j41}R^{k41}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{4A} are each optionally substituted with 1, 2, 3, 4, 5, 6, 7, or 8 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a41}, R^{c41} and R^{d41} are each optionally substituted with 1, 2, 3, 4, 5, 6, 7, or 8 independently selected R^{4B} substituents;
or, any R^{c41} and R^{d41} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4B} substituents;
each R^{b41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b41} are each optionally substituted with 1, 2, 3, 4, 5, 6, 7, or 8 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f41} and R^{g41} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h41} and Rⁱ⁴¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j41} and R^{k41} is independently selected from OH, C₁₋₆ alkoxy, and C₁₋₆ haloalkoxy;
or any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R⁴², OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)R^{b42}, NR^{c42}S(O)NR^{c42}R^{d42}, NR^{c42}S(O)₂R^{b42}, NR^{c42}S(O)(═NR^{e42})R^{b42}, NR^{c42}S(O)₂NR^{c42}R^{d42} S(O)R^{b42}, S(O)NR^{c42}R^{d42}, S(O)₂R^{b42}, S(O)₂NR^{c42}R^{d42}, OS(O)(═NR^{e42})R^{b42}, OS(O)₂R^{b42}, SF₅, P(O)R^{f42}R^{g42}, OP(O)(OR^{h42})(ORⁱ⁴²), P(O)(OR^{h42})(ORⁱ⁴²), and BR^{j42}R^{k42}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{4B} are each optionally substituted with 1, 2, 3, 4, 5, 6, 7, or 8 independently selected R^{4C} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a42}, R^{c42} and R^{d42} are each optionally substituted with 1, 2, 3, 4, 5, 6, 7, or 8 independently selected R^{4C} substituents;
or, any R^{c42} and R^{d42} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4C} substituents;
each R^{b42} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b42} are each optionally substituted with 1, 2, 3, 4, 5, 6, 7, or 8 independently selected R^{4C} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f42} and R^{g42} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h42} and Rⁱ⁴² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j42} and R^{k42} is independently selected from OH, C₁₋₆ alkoxy, and C₁₋₆ haloalkoxy;
or any R^{j42} and R^{k42} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a43}SR^{a43}, NHOR^{a43}, C(O)R^{b43}, C(O)NR^{c43}R^{d43}, C(O)NR^{c43}(OR^{a43}), C(O)OR^{a43}, OC(O)R^{b43}, OC(O)NR^{c43}R^{d43}, NR^{c43}R^{d43}, NR^{c43}NR^{c43}R^{d43}, NR^{c43}C(O)R^{b43}, NR^{c43}C(O)OR^{a43}, NR^{c43}C(O)NR^{c43}R^{d43}, C(═NR^{e43})R^{b43}, C(═NR^{e43})NR^{c43}R^{d43}, NR^{c43}C(═NR^{e43})NR^{c43}R^{d43}, NR^{c43}C(═NR^{e43})R^{b43}, NR^{c43}S(O)R^{b43}, NR^{c43}S(O)NR^{c43}R^{d43}, NR^{c43}S(O)₂R^{b43}, NR^{c43}S(O)(═NR^{e43})R^{b43}, NR^{c43}S(O)₂NR^{c43}R^{d43}, S(O)R^{b43}, S(O)NR^{c43}R^{d43}, S(O)₂R^{b43}, S(O)₂NR^{c43}R^{d43}, OS(O)(═NR^{e43})R^{b43}, and OS(O)₂R^{b43}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{4C} are each optionally substituted with 1, 2, 3, or 4 independently selected R^{M} substituents;
each R^{a43}, R^{c43}, and R^{d43} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a43}, R^{c43} and R^{d43} are each optionally substituted with 1, 2, 3, or 4 independently selected R^{M} substituents;
or, any R^{c43} and R^{d43} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b43} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b43} are each optionally substituted with 1, 2, 3, or 4 independently selected R^{M} substituents;
each R^{c43} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
X¹ is absent or is selected from C₁₋₆ alkylene, C₁₋₆ alkoxylene, C₂₋₆ alkynylene, C₃₋₁₀ cycloalkylene, 4-7 membered heterocycloalkylene, —C(O)—, —CH₂C(O)—, —O—, —NR^{NX1}— and —NR^{NX1}C(O)—;
R^{NX1} is selected from H and C₁₋₆ alkyl;
L is a linker;
E is an E3 ubiquitin ligase binding moiety (e.g., a binding moiety which is capable of binding to an E3 ubiquitin ligase); and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments, R¹ is selected from H, C₁₋₆ alkyl, C₂₋₆ alkenyl, and C₂₋₆ alkynyl.

In some embodiments, R¹ is selected from H and C₁₋₆ alkyl.

In some embodiments, R¹ is C₁₋₆ alkyl.

In some embodiments, R¹ is C₁₋₃ alkyl.

In some embodiments, R¹ is methyl or trideuteromethyl.

In some embodiments, R¹ is methyl.

In some embodiments, R¹ is trideuteromethyl.

In some embodiments, R² is selected from C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents.

In some embodiments, R² is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents.

In some embodiments, R² is selected from C₁₋₆ alkyl, phenyl, C₃₋₇ cycloalkyl, 5-6 membered heteroaryl, 4-10 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R² are each optionally substituted with 1, 2, 3, or 4 independently selected R^{2A} substituents.

In some embodiments, R² is selected from phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R² are each optionally substituted with 1, 2, 3, or 4 independently selected R^{2A} substituents.

In some embodiments, R² is selected from C₁₋₆ alkyl, phenyl, C₃₋₇ cycloalkyl, 5-6 membered heteroaryl, and 4-7 membered heterocycloalkyl, wherein the C₁₋₆ alkyl, phenyl, C₃₋₇ cycloalkyl, 5-6 membered heteroaryl, and 4-7 membered heterocycloalkyl of R² are each optionally substituted with 1, 2, 3, or 4 independently selected R^{2A} substituents.

In some embodiments, R² is selected from phenyl, C₃₋₇ cycloalkyl, 5-6 membered heteroaryl, and 4-10 membered heterocycloalkyl, wherein the phenyl, C₃₋₇ cycloalkyl, 5-6 membered heteroaryl, and 4-10 membered heterocycloalkyl of R² are each optionally substituted with 1, 2, 3, or 4 independently selected R^{2A} substituents.

In some embodiments, R² is selected from C₁₋₆ alkyl, C₃₋₇ cycloalkyl, and 4-10 membered heterocycloalkyl, wherein the C₁₋₆ alkyl, C₃₋₇ cycloalkyl, and 4-10 membered heterocycloalkyl ofR² are each optionally substituted with 1, 2, 3, or 4 independently selected R^{2A} substituents.

In some embodiments, R² is C₁₋₆ alkyl, which is optionally substituted with 1, 2, 3, or 4 independently selected R^{2A} substituents.

In some embodiments, R² is isopropyl, which is optionally substituted with 1, 2, 3, or 4 independently selected R^{2A} substituents.

In some embodiments, R² is C₃₋₁₀ cycloalkyl, which is optionally substituted with 1, 2, 3, or 4 independently selected R^{2A} substituents.

In some embodiments, R² is C₃₋₇ cycloalkyl, which is optionally substituted with 1, 2, 3, or 4 independently selected R^{2A} substituents.

In some embodiments, R² is cyclopentyl, which is optionally substituted with 1, 2, 3, or 4 independently selected R^{2A} substituents.

In some embodiments, R² is cyclopentyl, which is optionally substituted with 1 or 2 independently selected R^{2A} substituents.

In some embodiments, R² is cyclopentyl, which is optionally substituted with 1 R^{2A} substituent.

In some embodiments, R² is cyclopentyl, which is substituted with 1 R^{2A} substituent.

In some embodiments, R² is

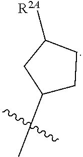

In some embodiments, R² is cyclopropyl, which is optionally substituted with 1, 2, 3, or 4 independently selected R^{2A} substituents.

In some embodiments, R² is 4-10 membered heterocycloalkyl, which is optionally substituted with 1, 2, 3, or 4 independently selected R^{2A} substituents.

In some embodiments, R² is 4-8 membered heterocycloalkyl, which is optionally substituted with 1, 2, 3, or 4 independently selected R^{2A} substituents.

In some embodiments, R² is tetrahydrofuran, which is optionally substituted with 1, 2, 3, or 4 independently selected R^{2A} substituents.

In some embodiments, R² is tetrahydropyran, spiro-tetrahydropyran, bridged bicycle containing tetrahydropyran, and fused bicycle containing tetrahydropyran, which is optionally substituted with 1, 2, 3, or 4 independently selected R^{2A} substituents.

In some embodiments, each R^{2A} is independently selected from C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21} NR^{c21}NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)NR^{c21}R^{d21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and OS(O)₂R^{b21}.

In some embodiments, each R^{2A} is independently selected from NR^{c21}C(O)R^{b21}, NR^{c21}C(O)OR^{a21} and NR^{c21}C(O)NR^{c21}R^{d21}.

In some embodiments, each R^{2A} is independently selected from NR^{c21}C(O)OR^{a21}.

In some embodiments, each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl.

In some embodiments, each R^{a21}, R^{b21}, R^{c21}, and R^{d21} is independently selected from H, C₁₋₆ alkyl, and C₁₋₆ haloalkyl.

In some embodiments, each R^{a21}, R^{b21}, R^{c21}, and R^{d21} is independently selected from H and C₁₋₆ alkyl.

In some embodiments, each R^{a21}, R^{b21}, R^{c21}, and R^{d21} is independently selected from H and C₁₋₃ alkyl.

In some embodiments, each R^{a21} and R^{c21} is independently selected from H, C₁₋₆ alkyl, C₁₋₆ haloalkyl, C₂₋₆ alkenyl, and C₂₋₆ alkynyl.

In some embodiments, each R^{a21} and R^{c21} is independently selected from H, C₁₋₆ alkyl, and C₁₋₆ haloalkyl.

In some embodiments, each R^{a21} and R^{c21} is independently selected from H and C₁₋₆ alkyl.

In some embodiments, each R^{a21} and R^{c21} is independently selected from H and C₁₋₃ alkyl.

In some embodiments, each R^{a21} and R^{c21} is independently selected from H and methyl.

In some embodiments, each R^{2A} is independently selected from C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)NR^{c21}R^{d21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and OS(O)₂R^{b21}; and
each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl.

In some embodiments, each R^{2A} is independently selected from NR^{c21}C(O)R^{b21}, NR^{c21}C(O)OR^{a21}, and NR^{c21}C(O)NR^{c21}R^{d21}; and
each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl.

In some embodiments, each R^{2A} is independently selected from NR^{c21}C(O)OR^{a21}; and
each R^{a21} and R^{c21} is independently selected from H, C₁₋₆ alkyl, C₁₋₆ haloalkyl, C₂-₆ alkenyl, and C₂₋₆ alkynyl.

In some embodiments, each R^{2A} is independently selected from NR^{c21}C(O)OR^{a21}; and
each R^{a21} and R^{c21} is independently selected from H and C₁₋₆ alkyl.

In some embodiments, each R^{2A} is independently selected from NHC(O)OR^{a21}; and
each R^{a21} is independently selected from C₁₋₆ alkyl.

In some embodiments, each R^{2A} is independently selected from NHC(O)OR^{a21}; and
each R^{a21} is independently selected from C₁₋₃ alkyl.

In some embodiments, each R^{2A} is NHC(O)OCH₃.

In some embodiments, R² is

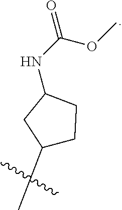 [SMILES: *C1CCC(NC(=O)OC)C1]

In some embodiments, R³ is selected from H, C₁₋₆ alkyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, or 4 independently selected R^{3A} substituents.

In some embodiments, R³ is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, or 4 independently selected R^{3A} substituents.

In some embodiments, R³ is H.

In some embodiments, R³ is selected from H, C₁₋₆ alkyl, C₆₋₁₀ aryl, C₃₋₁₀ cycloalkyl, 5-10 membered heteroaryl, and 4-10 membered heterocycloalkyl, wherein the C₁₋₆ alkyl, C₆₋₁₀ aryl, C₃₋₁₀ cycloalkyl, 5-10 membered heteroaryl, and 4-10 membered heterocycloalkyl of R³ are each optionally substituted with 1, 2, 3, or 4 independently selected R^{3A} substituents.

In some embodiments, R³ is selected from C₆₋₁₀ aryl, C₃₋₁₀ cycloalkyl, 5-10 membered heteroaryl, and 4-10 membered heterocycloalkyl, wherein the C₆₋₁₀ aryl, C₃₋₁₀ cycloalkyl, 5-10 membered heteroaryl, and 4-10 membered heterocycloalkyl of R³ are each optionally substituted with 1, 2, 3, or 4 independently selected R^{3A} substituents.

In some embodiments, R³ is selected from C₆₋₁₀ aryl, 5-10 membered heteroaryl, and 4-10 membered heterocycloalkyl, wherein the C₆₋₁₀ aryl, 5-10 membered heteroaryl, and 4-10 membered heterocycloalkyl of R³ are each optionally substituted with 1, 2, 3, or 4 independently selected R^{3A} substituents.

In some embodiments, R³ is selected from phenyl, 8-10 membered heteroaryl, and 8-10 membered heterocycloalkyl, wherein the phenyl, 8-10 membered heteroaryl, and 8-10 membered heterocycloalkyl of R³ are each optionally substituted with 1, 2, 3, or 4 independently selected R^{3A} substituents.

In some embodiments, R³ is selected from phenyl, bicyclic 8-10 membered heteroaryl, and bicyclic 8-10 membered heterocycloalkyl, wherein the phenyl, bicyclic 8-10 membered heteroaryl, and bicyclic 8-10 membered heterocycloalkyl of R³ are each optionally substituted with 1, 2, 3, or 4 independently selected R^{3A} substituents.

In some embodiments, R³ is selected from phenyl, indazolyl, and dihydroisobenzofuranyl, wherein the phenyl, indazolyl, and dihydroisobenzofuranyl of R³ are each optionally substituted with 1, 2, 3, or 4 independently selected R^{3A} substituents.

In some embodiments R³ is selected from

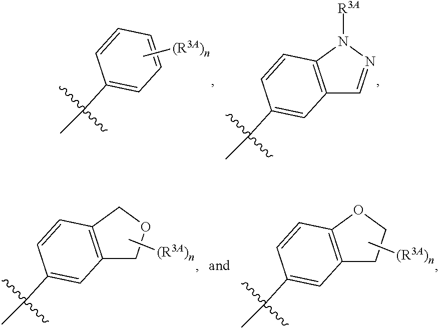

wherein n is 0, 1, 2, 3, or 4.

In some embodiments, R³ is selected from

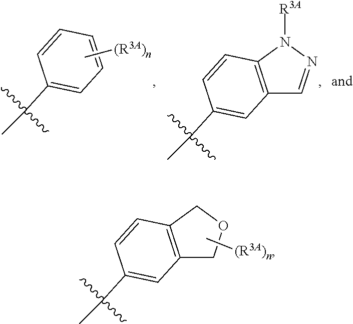

wherein n is 0, 1, 2, 3, or 4.

In some embodiments, R³ is selected from

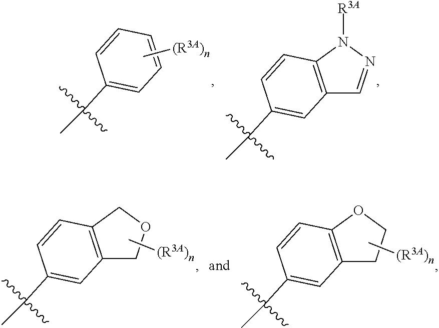

and, wherein n is 0, 1, or 2.

In some embodiments, R³ is selected from

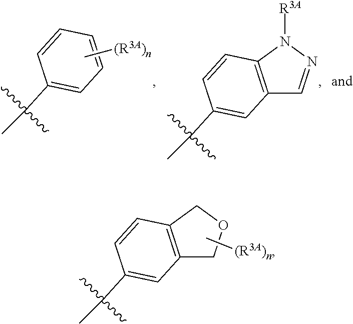

wherein n is 0, 1, or 2.

In some embodimetns, R³ is selected from

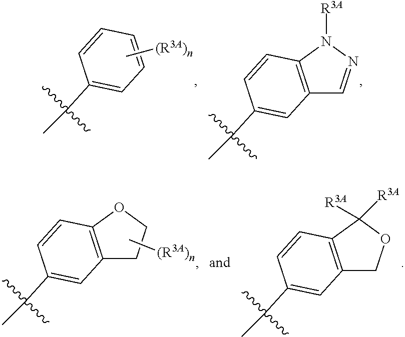

In some embodimetns, R³ is selected from

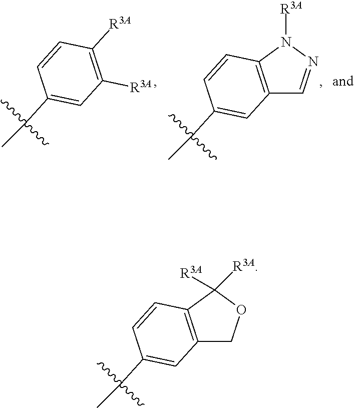

In some embodiments, each R^{3A} is independently selected from halo, oxo, C₁₋₆ alkyl, C₁₋₆ haloalkyl, C₂₋₆ alkenyl, C₂₋₆ alkynyl, and OR^{a31}.

In some embodiments, each R^{3A} is independently selected from halo, C₁₋₆ alkyl, C₁₋₆ haloalkyl, and OR^{a31}.

In some embodiments, each R^{3A} is independently selected from halo, C₁₋₆ alkyl, and OR^{a31}.

In some embodiments, each R^{a31}, R^{b31}, R^{c31}, and R^{d31} is independently selected from H, C₁₋₆ alkyl, C₁₋₆ haloalkyl, C₂₋₆ alkenyl, and C₂₋₆ alkynyl.

In some embodiments, each R^{a31}, R^{b31}, R^{c31}, and R^{d31} is independently selected from H, C₁₋₆ alkyl, and C₁₋₆ haloalkyl.

In some embodiments, each R^{a31}, R^{b31}, R^{c31}, and R^{d31} is independently selected from H and C₁₋₆ alkyl.

In some embodiments, each R^{a31}, R^{b31}, R^{c31}, and R^{d31} is independently selected from H and C₁₋₃ alkyl.

In some embodiments, each R^{a31} is independently selected from H, C₁₋₆ alkyl, C₁₋₆ haloalkyl, C₂₋₆ alkenyl, and C₂₋₆ alkynyl.

In some embodiments, each R^{a31} is independently selected from H, C₁₋₆ alkyl, and C₁₋₆ haloalkyl.

In some embodiments, each R^{a31} is independently selected from H and C₁₋₆ alkyl.

In some embodiments, each R^{a31} is independently selected from H and C₁₋₃ alkyl.

In some embodiments, each R^{a31} is methyl.

In some embodiments, each R^{3A} is independently selected from halo, oxo, C₁₋₆ alkyl, C₁₋₆ haloalkyl, C₂₋₆ alkenyl, C₂₋₆ alkynyl, and OR^{a31}; and
each R^{a31} is independently selected from H, C₁₋₆ alkyl, C₁₋₆ haloalkyl, C₂₋₆ alkenyl, and C₂₋₆ alkynyl.

In some embodiments, each R^{3A} is independently selected from halo, C₁₋₆ alkyl, C₁₋₆ haloalkyl, and OR^{a31}; and
each R^{a31} is independently selected from H and C₁₋₆ alkyl.

In some embodiments, each R^{3A} is independently selected from methyl, isopropyl, fluoro, and methoxy.

In some embodiments, R³ is selected from

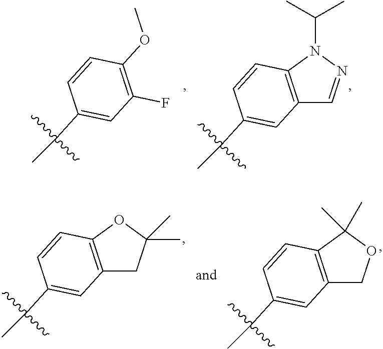

In some embodiments, R³ is selected from

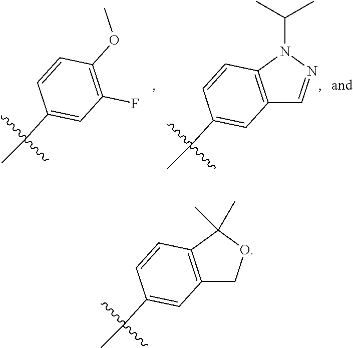

In some embodiments, R³ is

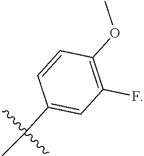 [SMILES: *c1ccc(OC)c(F)c1]

In some embodiments, R³ is

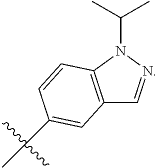 [SMILES: *c1ccc2c(cnn2C(C)C)c1]

In some embodiments, R³ is

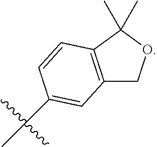 [SMILES: *c1ccc2c(c1)COC2(C)C]

In some embodiments, R³ is

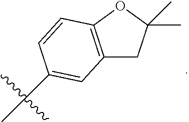 [SMILES: *c1ccc2c(c1)CC(C)(C)O2]

In some embodiments, R⁴ is selected from C₂₋₆ alkynylene, C₆₋₁₀ arylene, C₃₋₁₀ cycloalkylene, 5-10 membered heteroarylene, 4-10 membered heterocycloalkylene, —C₆₋₁₀ arylene-C₁₋₆ alkylene-, —C₃₋₁₀ cycloalkylene-C₁₋₆ alkylene-, -(5-10 membered heteroarylene)-C₁₋₆ alkylene-, and -(4-10 membered heterocycloalkylene)-C₁₋₆ alkylene-, wherein the C₂₋₆ alkynylene, C₆₋₁₀ arylene, C₃₋₁₀ cycloalkylene, 5-10 membered heteroarylene, 4-10 membered heterocycloalkylene, —C₆₋₁₀ arylene-C₁₋₆ alkylene-, —C₃₋₁₀ cycloalkylene-C₁₋₆ alkylene-, -(5-10 membered heteroarylene)-C₁₋₆ alkylene-, and -(4-10 membered heterocycloalkylene)-C₁₋₆ alkylene- of R⁴ are each optionally substituted with 1, 2, 3, or 4 independently selected R^{4A} substituents.

In some embodiments, R⁴ is selected from C₆₋₁₀ arylene, C₃₋₁₀ cycloalkylene, 5-10 membered heteroarylene, 4-10 membered heterocycloalkylene, —C₆₋₁₀ arylene-C₁₋₆ alkylene-, —C₃₋₁₀ cycloalkylene-C₁₋₆ alkylene-, -(5-10 membered heteroarylene)-C₁₋₆ alkylene-, and -(4-10 membered heterocycloalkylene)-C₁₋₆ alkylene-, wherein the C₆₋₁₀ arylene, C₃₋₁₀ cycloalkylene, 5-10 membered heteroarylene, 4-10 membered heterocycloalkylene, —C₆₋₁₀ arylene-C₁₋₆ alkylene-, —C₃₋₁₀ cycloalkylene-C₁₋₆ alkylene-, -(5-10 membered heteroarylene)-C₁₋₆ alkylene-, and -(4-10 membered heterocycloalkylene)-C₁₋₆ alkylene- of R⁴ are each optionally substituted with 1, 2, 3, or 4 independently selected R^{4A} substituents.

In some embodiments, R⁴ is selected from C₆₋₁₀ arylene, C₃₋₁₀ cycloalkylene, 5-10 membered heteroarylene, and 4-10 membered heterocycloalkylene, wherein the C₆₋₁₀ arylene, C₃₋₁₀ cycloalkylene, 5-10 membered heteroarylene, and 4-10 membered heterocycloalkylene of R⁴ are each optionally substituted with 1, 2, 3, or 4 independently selected R^{4A} substituents.

In some embodiments, R⁴ is selected from phenylene, C₃₋₇ cycloalkylene, 5-6 membered heteroarylene, and 4-7 membered heterocycloalkylene, wherein the phenylene, C₃₋₇ cycloalkylene, 5-6 membered heteroarylene, and 4-7 membered heterocycloalkylene of R⁴ are each optionally substituted with 1, 2, 3, or 4 independently selected R^{4A} substituents.

In some embodiments, R⁴ is selected from phenylene and 5-6 membered heteroarylene, wherein the phenylene and 5-6 membered heteroarylene of R⁴ are each optionally substituted with 1, 2, 3, or 4 independently selected R^{4A} substituents.

In some embodiments, R⁴ is selected from phenylene and pyrazolylene, wherein the phenylene and 5-6 membered heteroarylene of R⁴ are each optionally substituted with 1, 2, 3, or 4 independently selected R^{4A} substituents.

In some embodiments, R⁴ is selected from 1,4-phenylene and 1,3-pyrazolylene, wherein the phenylene and 5-6 membered heteroarylene of R⁴ are each optionally substituted with 1, 2, 3, or 4 independently selected R^{4A} substituents.

In some embodiments, R⁴ is selected from

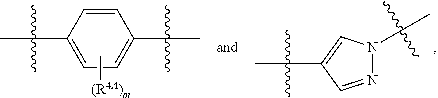

wherein m is 0, 1, 2, 3, or 4.

In some embodiments, R⁴ is selected from

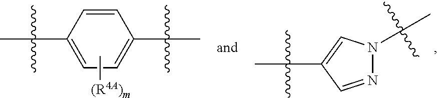

wherein m is 0, 1, or 2.

In some embodiments, R⁴ is selected from

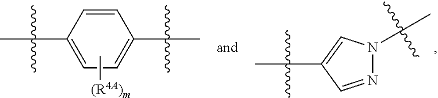

wherein m is 0 or 1.

In some embodiments, R⁴ is selected from

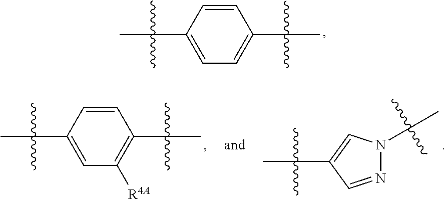

In some embodiments, each R^{4A} is independently selected from halo, C₁₋₆ alkyl, C₁₋₆ haloalkyl, C₂₋₆ alkenyl, C₂₋₆ alkynyl, and OR^{a41}.

In some embodiments, each R^{4A} is independently selected from halo, C₁₋₆ alkyl, C₁₋₆ haloalkyl, and OR^{a41}.

In some embodiments, each R^{4A} is independently selected from halo and OR^{a41}.

In some embodiments, each R^{a41}, R^{b41}, R^{c41}, and R^{d41} is independently selected from H, C₁₋₆ alkyl, C₁₋₆ haloalkyl, C₂₋₆ alkenyl, and C₂₋₆ alkynyl.

In some embodiments, each R^{a41}, R^{b41}, R^{c41}, and R^{d41} is independently selected from H, C₁₋₆ alkyl, and C₁₋₆ haloalkyl.

In some embodiments, each R^{a41}, R^{b41}, R^{c41}, and R^{d41} is independently selected from H and C₁₋₆ alkyl.

In some embodiments, each R^{a41}, R^{b41}, R^{c41}, and R^{d41} is independently selected from H and C₁₋₃ alkyl.

In some embodiments, each R^{a41} is independently selected from H, C₁₋₆ alkyl, C₁₋₆ haloalkyl, C₂₋₆ alkenyl, and C₂₋₆ alkynyl.

In some embodiments, each R^{a41} is independently selected from H, C₁₋₆ alkyl, and C₁₋₆ haloalkyl.

In some embodiments, each R^{a41} is independently selected from H and C₁₋₆ alkyl.

In some embodiments, each R^{a41} is independently selected from H and C₁₋₃ alkyl.

In some embodiments, each R^{a41} is methyl.

In some embodiments, each R^{4A} is independently selected from halo, C₁₋₆ alkyl, C₁₋₆ haloalkyl, C₂₋₆ alkenyl, C₂₋₆ alkynyl, and OR^{a41}; and
each R^{a41} is independently selected from H, C₁₋₆ alkyl, C₁₋₆ haloalkyl, C₂₋₆ alkenyl, and C₂₋₆ alkynyl.

In some embodiments, each R^{4A} is independently selected from halo and OR^{a41}; and
each R^{a41} is independently selected from H and C₁₋₆ alkyl.

In some embodiments, each R^{4A} is independently selected from halo and OR^{a41}; and
each R^{a41} is independently selected from H and C₁₋₃ alkyl.

In some embodiments, each R^{4A} is independently selected from fluoro and methoxy.

In some embodiments, R⁴ is selected from

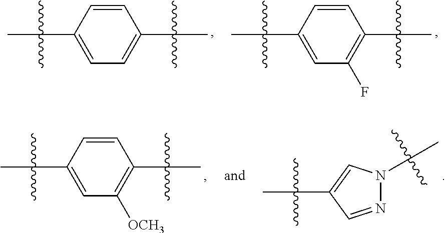 [SMILES: *c1ccc(*)cc1 ; *c1ccc(*)c(F)c1 ; *c1ccc(*)c(OC)c1 ; *c1cnn(*)c1]

In some embodiments, R⁴ is

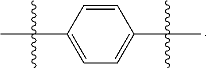 [SMILES: *c1ccc(*)cc1]

In some embodiments, R⁴ is

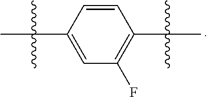 [SMILES: *c1ccc(*)c(F)c1]

In some embodiments, R⁴ is

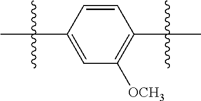 [SMILES: *c1ccc(*)c(OC)c1]

In some embodiments, R⁴ is

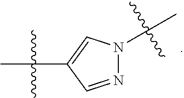 [SMILES: *c1cnn(*)c1]

In some embodiments, L is —W—R⁵—Y—X²—, wherein:
X² is absent or is selected from C₁₋₆ alkylene, C₁₋₆ alkoxylene, C₂₋₆ alkynylene, C₃₋₁₀ cycloalkylene, —C(O)—, —CH₂C(O)—, —NR^{NX2}—, and —NR^{NX2}C(O)—;
R^{NX2} is selected from H and C₁₋₆ alkyl;
W is absent or is selected from C₁₋₆ alkyl, C₁₋₆ alkoxy, C₁₋₆ haloalkyl, C₂₋₆ alkenyl, C₂₋₆ alkynyl, 4-11 membered heterocycloalkylene, 4-11 membered cycloalkylene, 5-10 membered heteroarylene, C₆₋₁₀ arylene wherein the 4-11 membered heterocycloalkylene, 4-11 membered cycloalkylene, 5-10 membered heteroarylene, C₆₋₁₀ arylene are each optionally substituted with 1, 2, 3, 4, 5, 6, 7, or 8 independently selected R^{e42} substituents;
Y is absent or is selected from C₁₋₆ alkyl, C₁₋₆ alkoxy, C₁₋₆ haloalkyl, C₂₋₆ alkenyl, C₂₋₆ alkynyl, 4-11 membered heterocycloalkylene, 4-11 membered cycloalkylene, 5-10 membered heteroarylene, C₆₋₁₀ arylene wherein the 4-11 membered heterocycloalkylene, 4-11 membered cycloalkylene, 5-10 membered heteroarylene, C₆₋₁₀ arylene are each optionally substituted with 1, 2, 3, 4, 5, 6, 7, or 8 independently selected R^{e42} substituents; and
R⁵ is absent or selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ alkylene-O—C₁₋₆ alkylene-, —C₁₋₆ alkylene-O—C₁₋₆ haloalkylene-O—C₁₋₆ alkylene-C(O)—, —C₁₋₆ alkylene-C₃₋₁₀ cycloalkylene-C(O)—, —C₁₋₆ alkylene-C₆₋₁₀ arylene-C(O)—, 4-11 membered heterocycloalkylene, 4-11 membered cycloalkylene, 5-10 membered heteroarylene, C₆₋₁₀ arylene, —C₁₋₆ alkylene-4-11 membered heterocycloalkylene, —C₁₋₆ alkylene-4-11 membered cycloalkylene, —C₁₋₆ alkylene-5-10 membered heteroarylene, and —C₁₋₆ alkylene-C₆₋₁₀ arylene.

In some embodiments, L is —W—R⁵—Y—X²—, wherein:
X² is absent or is selected from —CH₂—, C(O), —CH₂C(O)—, —NR^{NX2}—, and C(O)NR^{NX2}—.

W is absent or is selected from 4-11 membered heterocycloalkylene, 4-11 membered cycloalkylene, 5-10 membered heteroarylene, C₆₋₁₀ arylene wherein the 4-11 membered heterocycloalkylene, 4-11 membered cycloalkylene, 5-10 membered heteroarylene, C₆₋₁₀ arylene are each optionally substituted with 1, 2, 3, 4, 5, 6, 7, or 8 independently selected R^{e42} substituents;
Y is absent or is selected from 4-11 membered heterocycloalkylene, 4-11 membered cycloalkylene, 5-10 membered heteroarylene, C₆₋₁₀ arylene wherein the 4-11 membered heterocycloalkylene, 4-11 membered cycloalkylene, 5-10 membered heteroarylene, C₆₋₁₀ arylene are each optionally substituted with 1, 2, 3, 4, 5, 6, 7, or 8 independently selected R^{e42} substituents; and
R⁵ is absent or selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ alkylene-O—C₁₋₆ alkylene-, —C₁₋₆ alkylene-O—C₁₋₆ haloalkylene-O—C₁₋₆ alkylene-C(O)—, —C₁₋₆ alkylene-C₃₋₁₀ cycloalkylene-C(O)—, and —C₁₋₆ alkylene-C₆₋₁₀ arylene-C(O)—.

In some embodiments, X¹ is selected from —CH₂—, C(O), and —CH₂C(O)—.

In some embodiments, X¹ is —CH₂—.

In some embodiments, X¹ is C(O).

In some embodiments, X¹ is —CH₂C(O)—.

In some embodiments, X¹ is absent.

In some embodiments, L is a linker selected from 4-11 membered heterocycloalkyl, (4-11 membered heterocycloalkyl)-C₁₋₆ alkyl-N(R^{N1}), (4-11 membered heterocycloalkyl)-C₁₋₆ alkyl-C(O)N(R^{N1}), (4-11 membered heterocycloalkyl)-R⁵-(4-11 membered heterocycloalkyl), (4-11 membered heterocycloalkyl)-R⁵-(4-11 membered heterocycloalkyl)-N(R^{N1}), (4-11 membered heterocycloalkyl)-R⁵-(4-11 membered heterocycloalkyl)-C(O)N(R^{N1}), (4-11 membered cycloalkyl)-C₁₋₆ alkyl-N(R^{N1}), (4-11 membered cycloalkyl)-C₁₋₆ alkyl-C(O)N(R^{N1}), (4-11 membered cycloalkyl)-R⁵-(4-11 membered heterocycloalkyl), (4-11 membered cycloalkyl)-R⁵-(4-11 membered heterocycloalkyl)-N(R^{N1}), (4-11 membered cycloalkyl)-R⁵-(4-11 membered heterocycloalkyl)-C(O)N(R^{N1}), (4-11 membered cycloalkyl)-R⁵-(4-11 membered heterocycloalkyl)-C₁₋₆ alkoxy, (4-11 membered cycloalkyl)-R⁵-(4-11 membered cycloalkyl), (4-11 membered cycloalkyl)-R⁵-(4-11 membered cycloalkyl)-N(R^{N1}), (4-11 membered cycloalkyl)-R⁵-(4-11 membered cycloalkyl)-C(O)N(R^{N1}), 5-10 membered heteroaryl, (5-10 membered heteroaryl)-C₁₋₆ alkyl-N(R^{N1}), (5-10 membered heteroaryl)-C₁₋₆ alkyl-C(O)N(R^{N1}), (5-10 membered heteroaryl)-R⁵-(5-10 membered heterocycloalkyl), (5-10 membered heteroaryl)-R⁵-(5-10 membered heterocycloalkyl)-N(R^{N1}), (5-10 membered heteroaryl)-R⁵-(5-10 membered heterocycloalkyl)-C(O)N(R^{N1}) 5-10 membered aryl, (5-10 membered aryl)-C₁₋₆ alkyl-N(R^{N1}), (5-10 membered aryl)-C₁₋₆ alkyl-C(O)N(R^{N1}), (5-10 membered aryl)-R⁵-(5-10 membered heterocycloalkyl), (5-10 membered aryl)-R⁵-(5-10 membered heterocycloalkyl)-N(R^{N1}), (5-10 membered aryl)-R⁵-(5-10 membered heterocycloalkyl)-C(O)N(R^{N1}), —N(R^{N1})—R⁵—N(R^{N2})—, and —N(R^{N1})—R⁵—O—, —O—R⁵—O—.

In some embodiments, L is a linker selected from

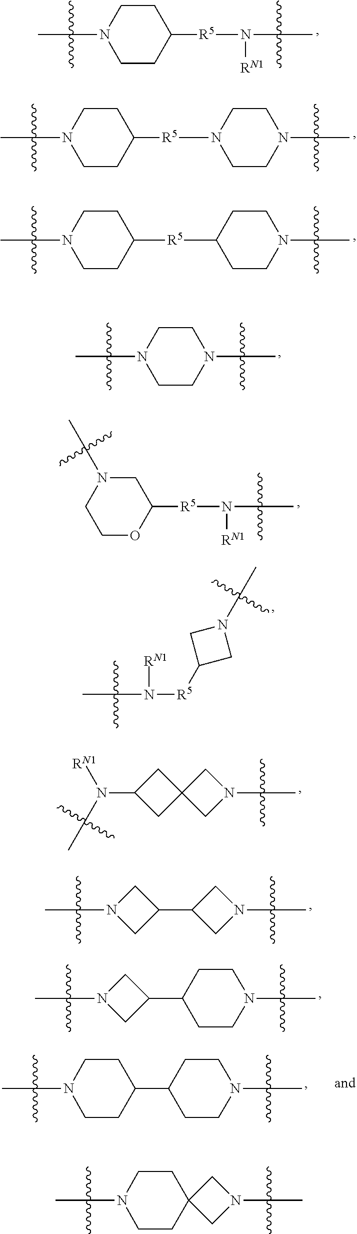

In some embodiments, L is a linker selected from

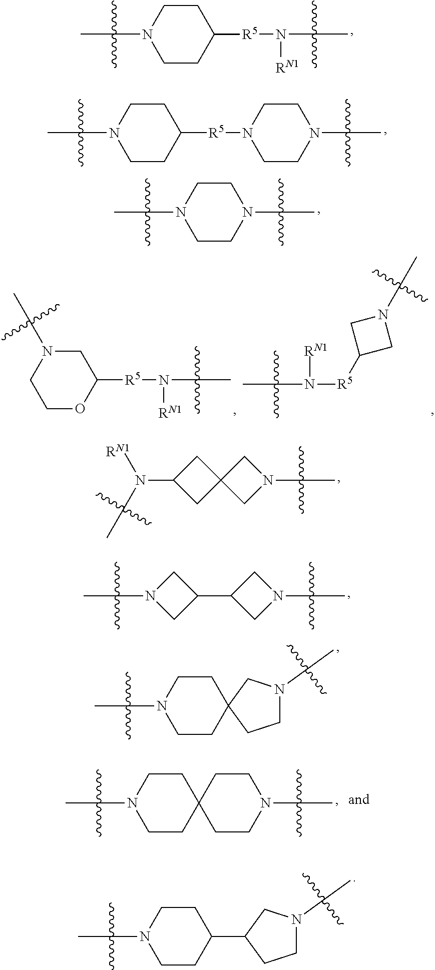

In some embodiments, L is a linker selected from —N(R^{N1})—R⁵—N(R^{N2})—, —N(R^{N1})—R⁵—O—,

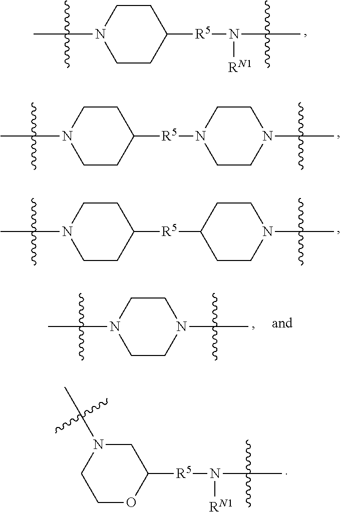

In some embodiments, L is a linker selected from —N(R^{N1})—R⁵—N(R^{N2})—, —N(R^{N1})—R⁵—O—,

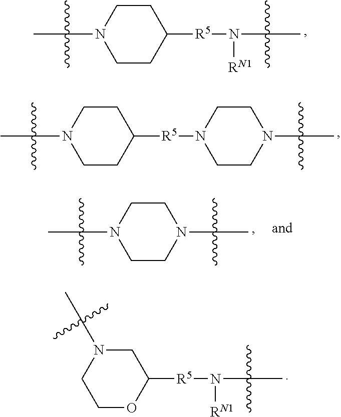

In some embodiments, R^{N1} is selected from H and C₁₋₃ alkyl.

In some embodiments, R^{N1} is selected from H and methyl.

In some embodiments, R^{N1} is H.

In some embodiments, R^{N1} is methyl.

In some embodiments, R^{N2} is selected from H and C₁₋₃ alkyl.

In some embodiments, R^{N2} is selected from H and methyl.

In some embodiments, R^{N2} is H.

In some embodiments, R^{N2} is methyl.

In some embodiments, R^{N1} is selected from H and C₁₋₃ alkyl; and R^{N2} is H.

In some embodiments, R^{N1} is selected from H and methyl; and R^{N2} is H.

In some embodiments, R⁵ is absent or selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ alkylene-O—C₁₋₆ alkylene-, —C₁₋₆ alkylene-O—C₁₋₆ haloalkylene-O—C₁₋₆ alkylene-C(O)—, —C₁₋₆ alkylene-C₃₋₁₀ cycloalkylene-C(O)—, and —C₁₋₆ alkylene-C₆₋₁₀ arylene-C(O)—.

In some embodiments, R⁵ is selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ haloalkylene-O—C₁₋₆ alkylene-C(O)—, —C₁₋₆ alkylene-C₃₋₁₀ cycloalkylene-C(O)—, and —C₁₋₆ alkylene-C₆₋₁₀ arylene-C(O)—.

In some embodiments, R⁵ is selected from —C(O)—, —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂C(O)—, —CH₂CH₂CH₂CH₂CH₂CH₂C(O)—, —CH₂CH₂CH₂CH₂—O—CH₂CF₂CH₂—O—CH₂C(O)—, —CH₂CH₂—O—CH₂CH₂—, —CH₂CH₂—O—CH₂CH₂—O—CH₂C(O)—, —CH₂CH₂—O—CH₂CH₂—O—CH₂CH₂C(O)—, —CH₂CH₂—O—CH₂C(O)—, —CH₂CH₂—O—CH₂CH₂C(O)—,

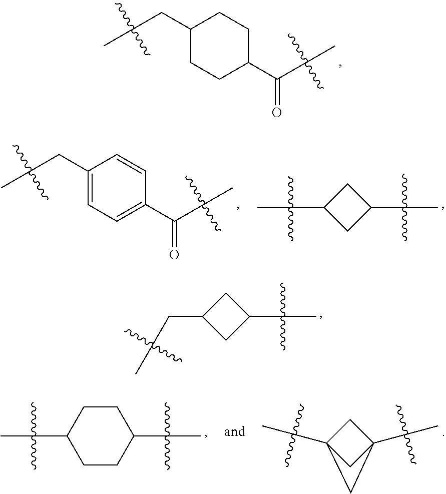 [SMILES: *CC1CCC(C(*)=O)CC1 ; *Cc1ccc(C(*)=O)cc1 ; *C1CC(*)C1 ; *CC1CC(*)C1 ; *C1CCC(*)CC1 ; *C12CC(*)(C1)C2]

In some embodiments, R⁵ is selected from —C(O)—, —CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂C(O)—, —CH₂CH₂CH₂CH₂CH₂CH₂C(O)—, —CH₂CH₂CH₂CH₂—O—CH₂CF₂CH₂—O—CH₂C(O)—, —CH₂CH₂—O—CH₂CH₂—, —CH₂CH₂—O—CH₂CH₂—O—CH₂C(O)—, —CH₂CH₂—O—CH₂CH₂—O—CH₂CH₂C(O)—, —CH₂CH₂—O—CH₂C(O)—, —CH₂CH₂—O—CH₂CH₂C(O)—,

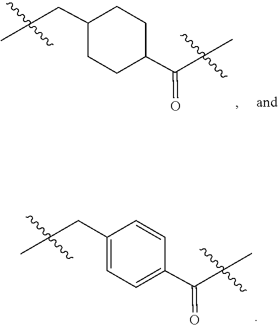 [SMILES: *CC1CCC(C(*)=O)CC1 ; *Cc1ccc(C(*)=O)cc1]

In some embodiments, L is selected from:

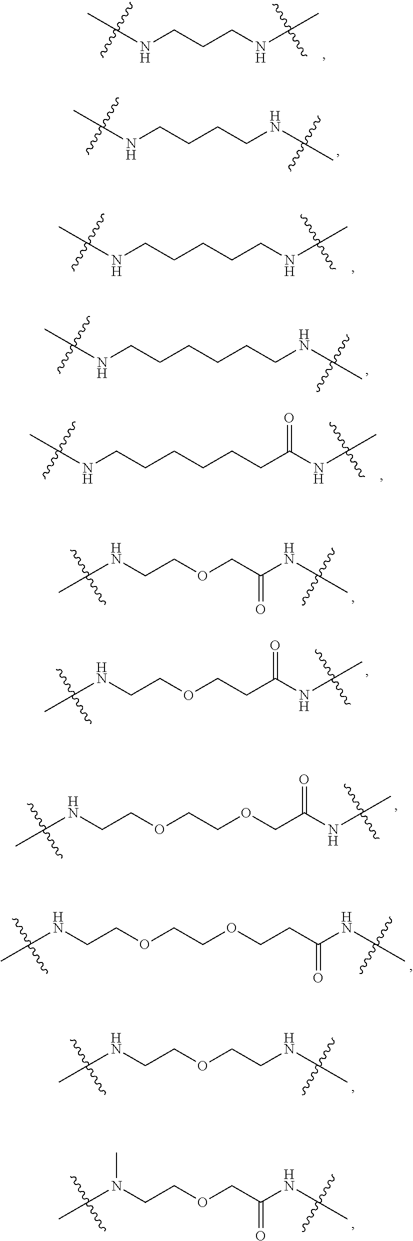

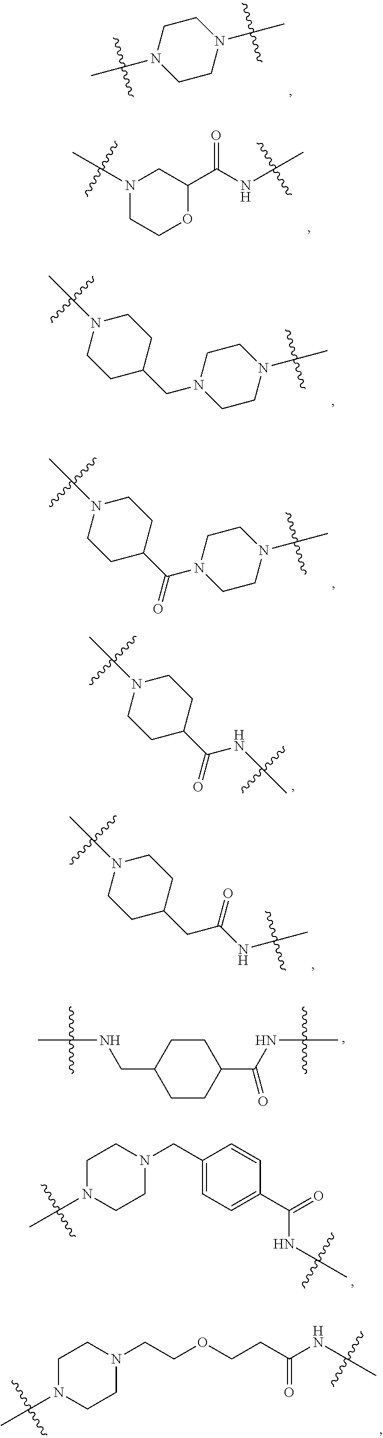

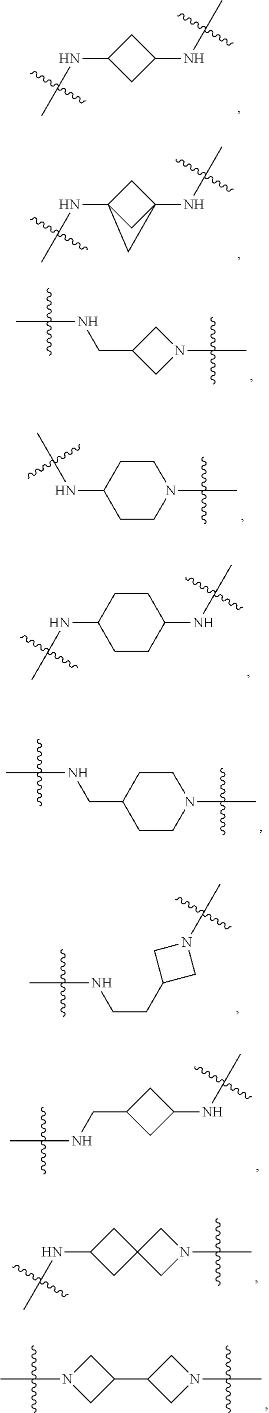

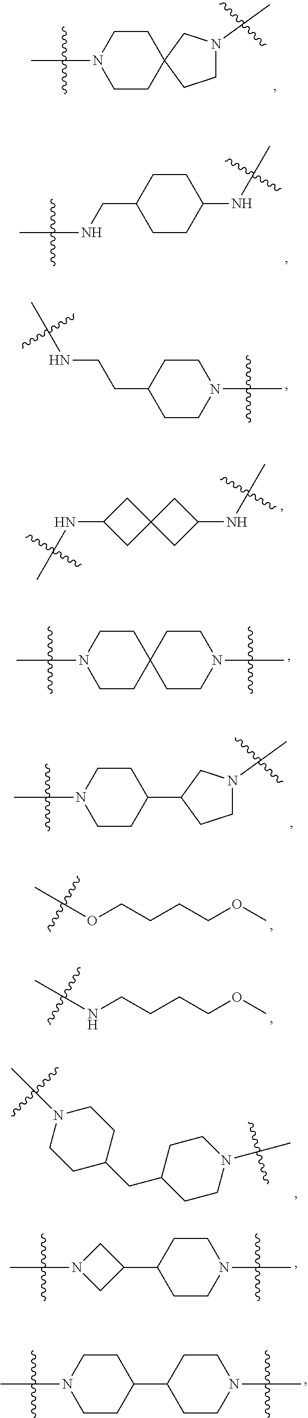

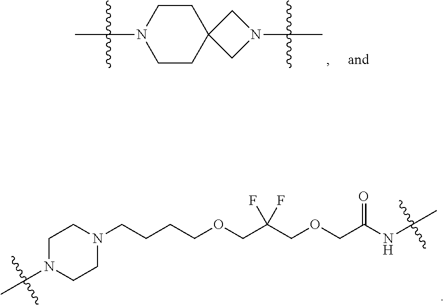 [SMILES: *N1CCC2(CC1)CN(*)C2 ; *NC(=O)COCC(F)(F)COCCCCN1CCN(*)CC1]

In some embodiments, L is selected from:

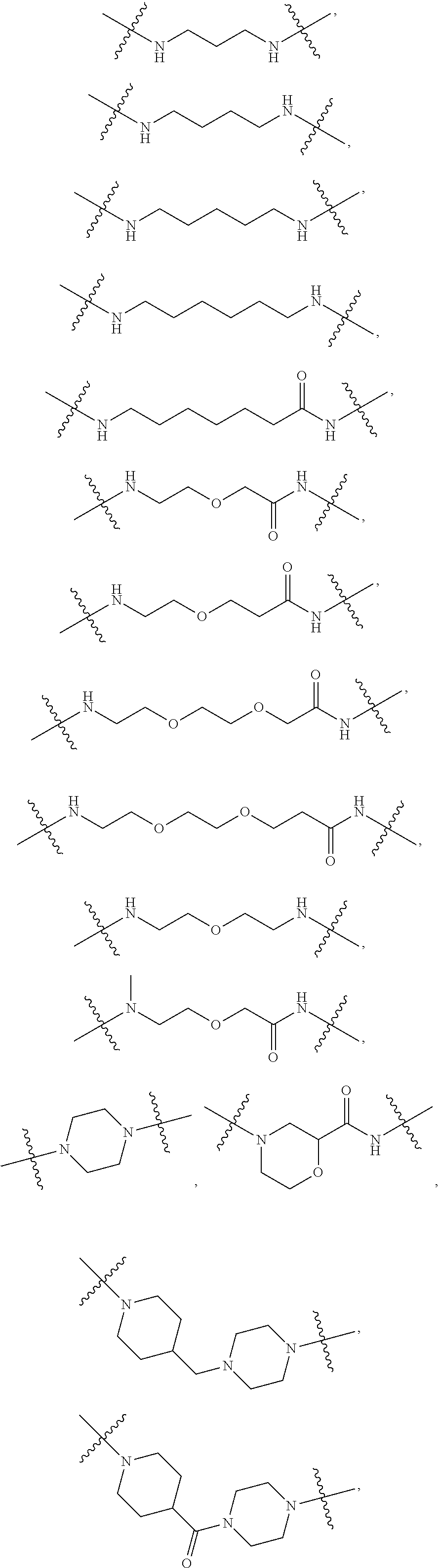

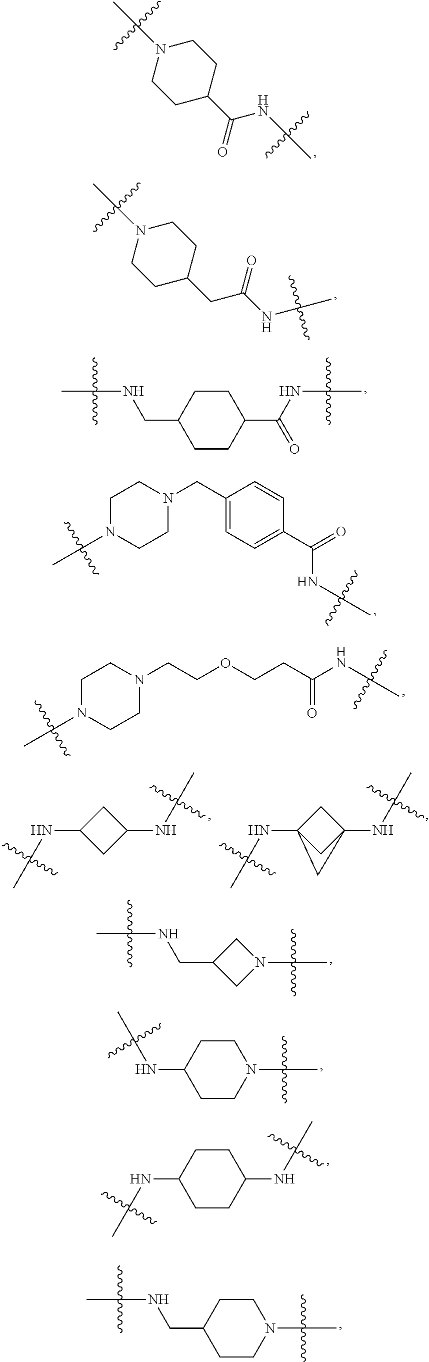

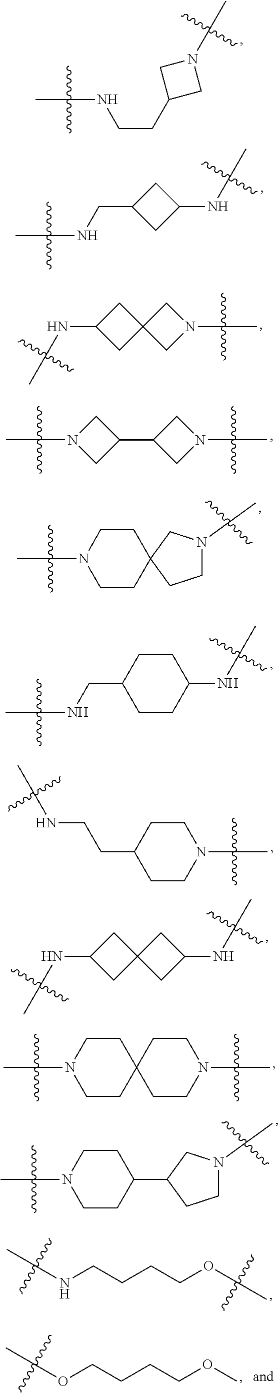

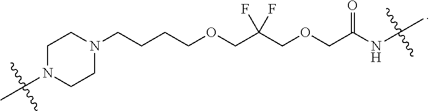 [SMILES: *NC(=O)COCC(F)(F)COCCCCN1CCN(*)CC1]

In some embodiments, L is selected from:

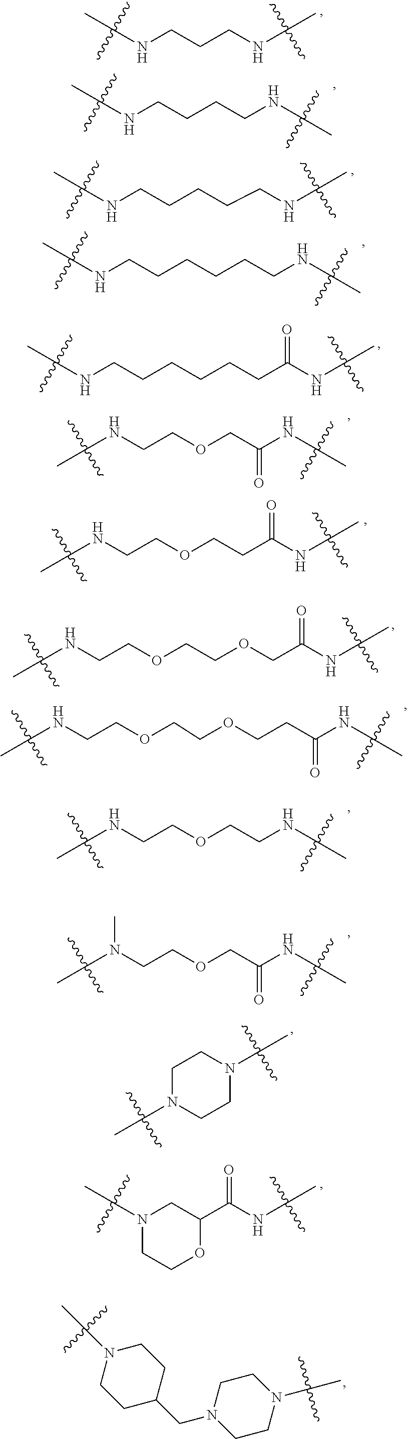

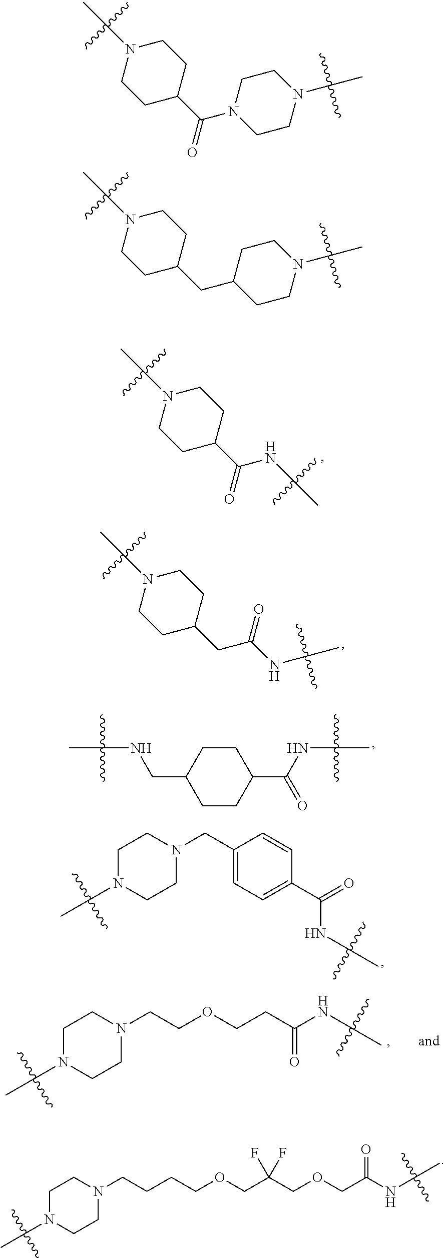

In some embodiments, L is selected from:

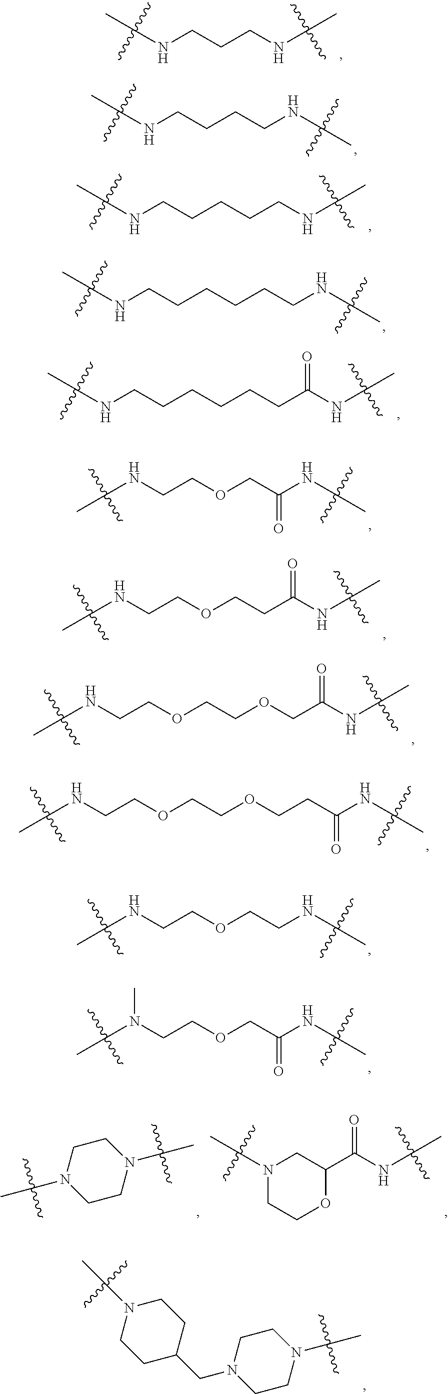

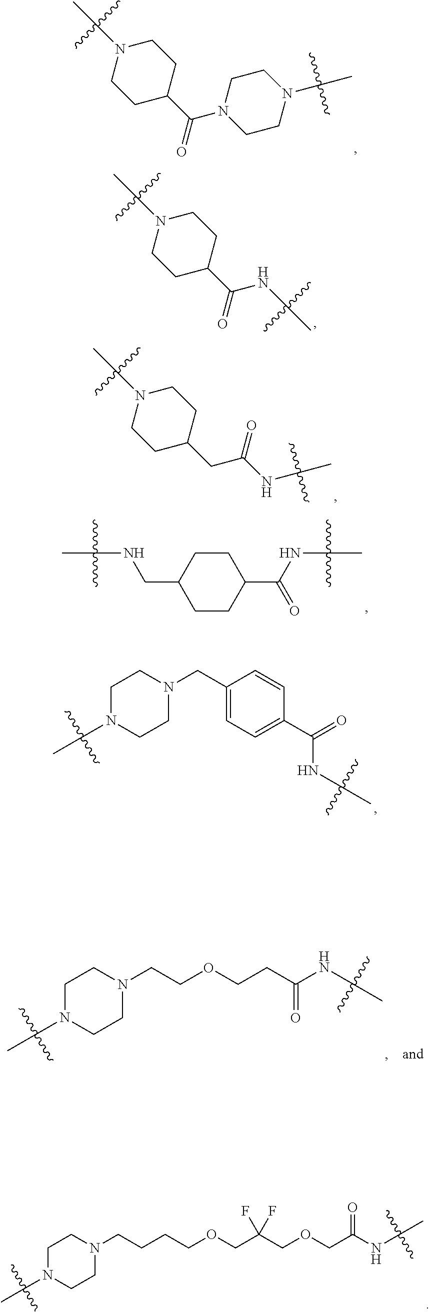

In some embodiments, E is an E3 ubiquitin ligase binding moiety. Ubiquitin ligase binding moieties are known and well-described in the art. Example E3 ubiquitin ligase binding moieties can be found, for example in Sosic, I. et al. Front. Chem. 2021, 9:707317; Han, X, Sun, Y. MedComm. 2023; 4:e290; Ma, Z. et al. Eur. J. Med. Chem. 2021, 216, 113247; Kannt, A; Dikic, I. Cell Chem. Biol. 2021, 28, 1014, and International Application Publication WO 2020/200291A1, the disclosure of each of which is incorporated herein by reference in its entirety. Example E3 ligase recruiters (E) can be purchased from various compound libraries, including, but not limited to, Biotechne/Tocris' PROTAC® Degraders & Targeted Protein Degradation library, Enamine's Protein Degrader Toolbox library, Millipore Sigma's (Aldrich) Targeted Protein Degradation libraries, or can be prepared using methods known by one skilled in the art, such as those described, for example, in Bricelj, A. et al. Front. Chem. 2021, 9, 707317 and International Application Publication No.: WO 2020/2000291A1, the disclosure of each of which is incorporated herein by reference in its entirety.

In some embodiments, E is selected from an E3 ubiquitin ligase binding moiety such as, but not limited to, Von Hippel-Lindau (VHL), mouse double minute 2 (MDM2), cereblon (CRBN), an inhibitor of apoptosis proteins (IAP), RING-H2 finger (RNF), aryl hydrocarbon receptor (AhR), DDB1-associated and Cul4-associated factor (DCAF), Kelch-like ECH-associated protein 1 (KEAP1), ring finger protein 114 (RNF₁₁₄), ring finger protein 4 (RNF₄), ring finger protein 43 (RNF₄₃), ring finger protein 7 (RNF₇) baculoviral IAP repeat containing 3 (BIRC₃), and RBBP7.

In some embodiments, E is a Von Hippel-Lindau (VHL) E3 ubiquitin ligase binding moiety.

In some embodiments, E is a MDM2 E3 ubiquitin ligase binding moiety.

In some embodiments, E is a cereblon E3 ubiquitin ligase binding moiety.

In some embodiments, E is an inhibitor of apoptosis protein (IAP) E3 ubiquitin ligase binding moiety.

In some embodiments, E is selected from:

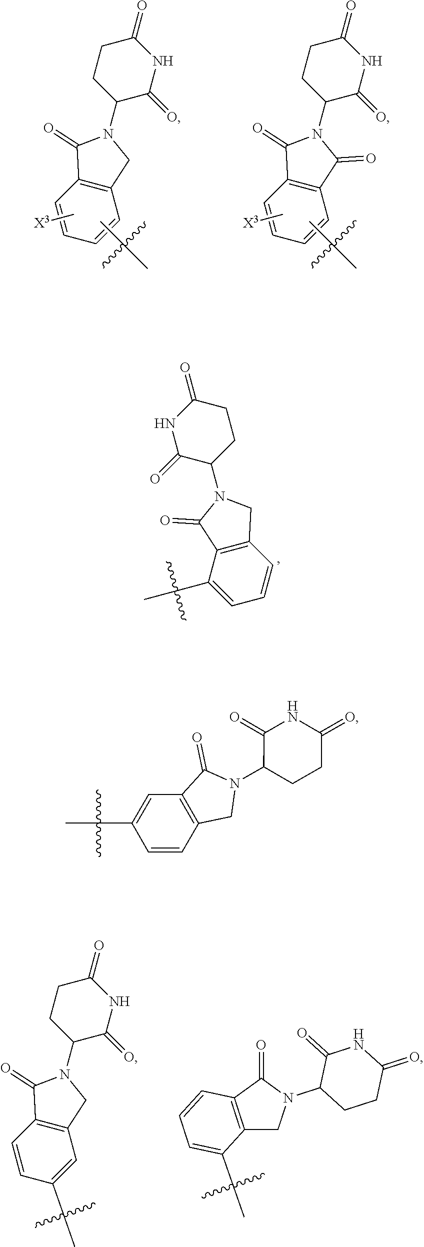

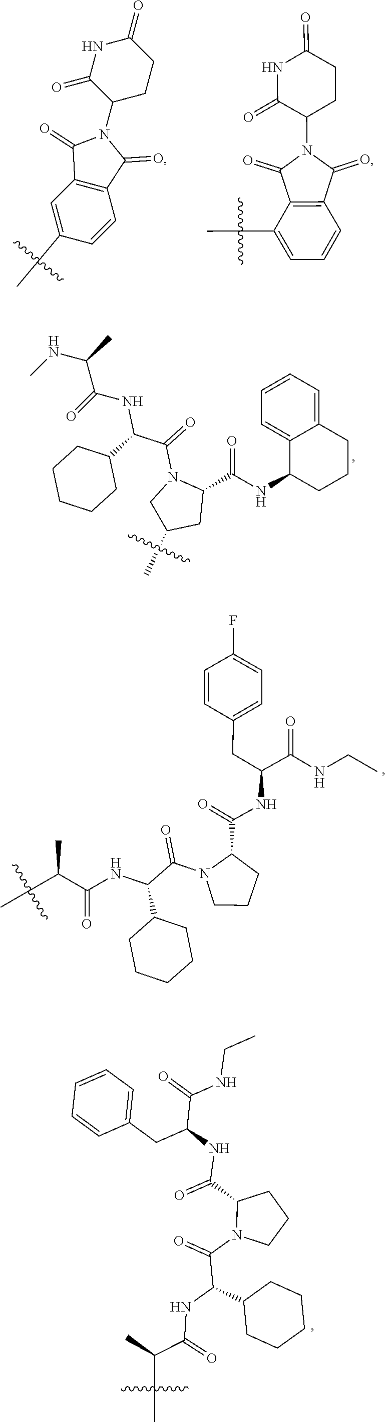

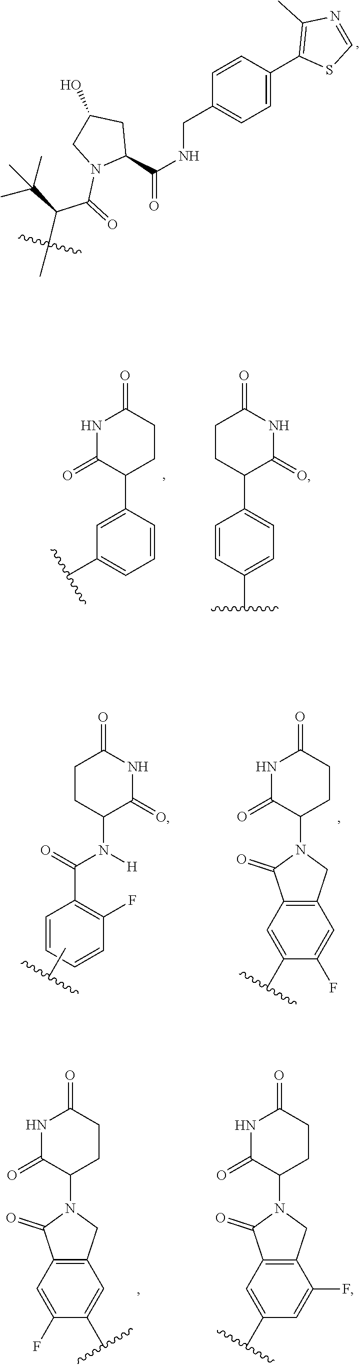

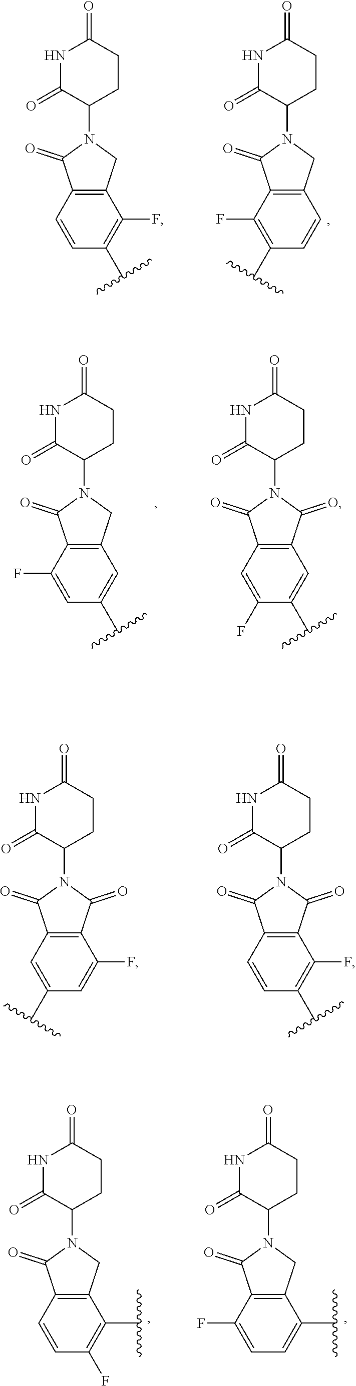

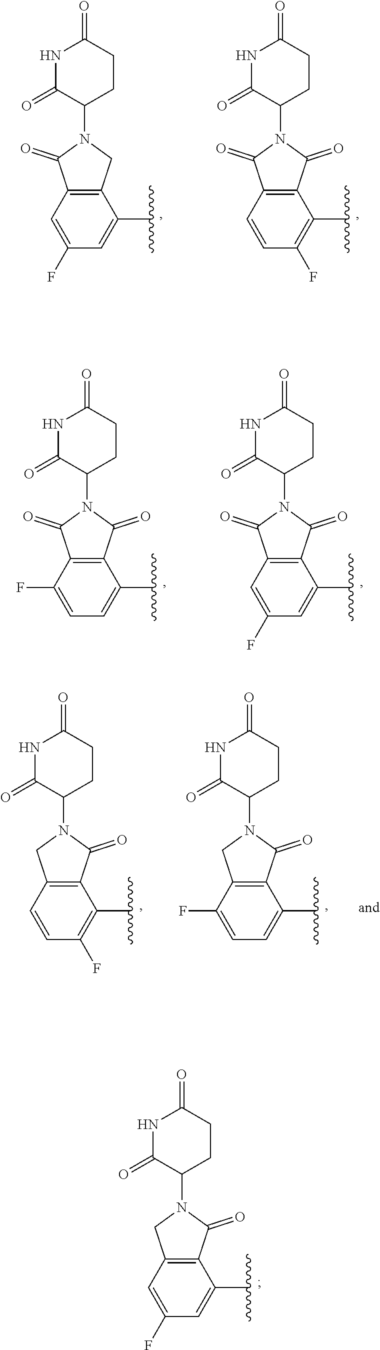

wherein X³ is selected from H and halo.

In some embodiments, E is selected from:

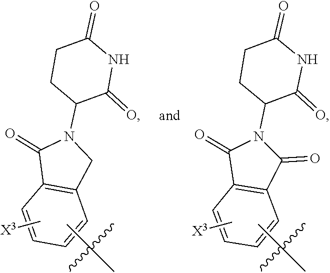

wherein X³ is selected from H and halo.

In some embodiments, E is selected from:

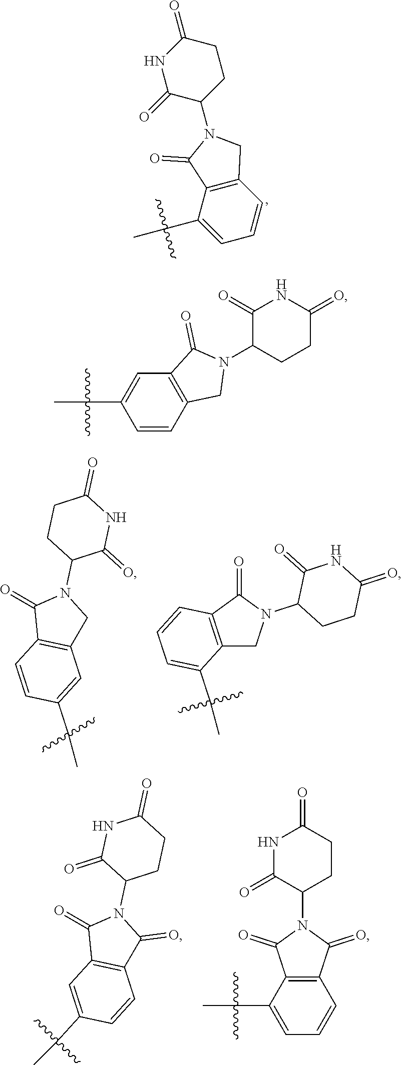 [SMILES: *c1cccc2c1C(=O)N(C1CCC(=O)NC1=O)C2 ; *c1ccc2c(c1)C(=O)N(C1CCC(=O)NC1=O)C2 ; *c1ccc2c(c1)CN(C1CCC(=O)NC1=O)C2=O ; *c1cccc2c1CN(C1CCC(=O)NC1=O)C2=O ; *c1ccc2c(c1)C(=O)N(C1CCC(=O)NC1=O)C2=O ; *c1cccc2c1C(=O)N(C1CCC(=O)NC1=O)C2=O]

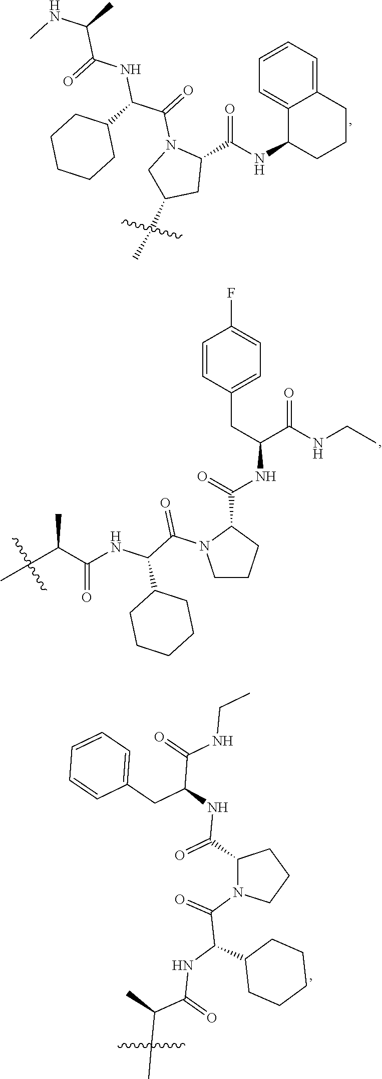 [SMILES: *[C@H]1C[C@@H](C(=O)N[C@@H]2CCCc3ccccc32)N(C(=O)[C@@H](NC(=O)[C@H](C)NC)C2CCCCC2)C1 ; *[C@@H](C)C(=O)N[C@H](C(=O)N1CCC[C@H]1C(=O)N[C@@H](Cc1ccc(F)cc1)C(=O)NCC)C1CCCCC1 ; *[C@@H](C)C(=O)N[C@H](C(=O)N1CCC[C@H]1C(=O)N[C@@H](Cc1ccccc1)C(=O)NCC)C1CCCCC1]

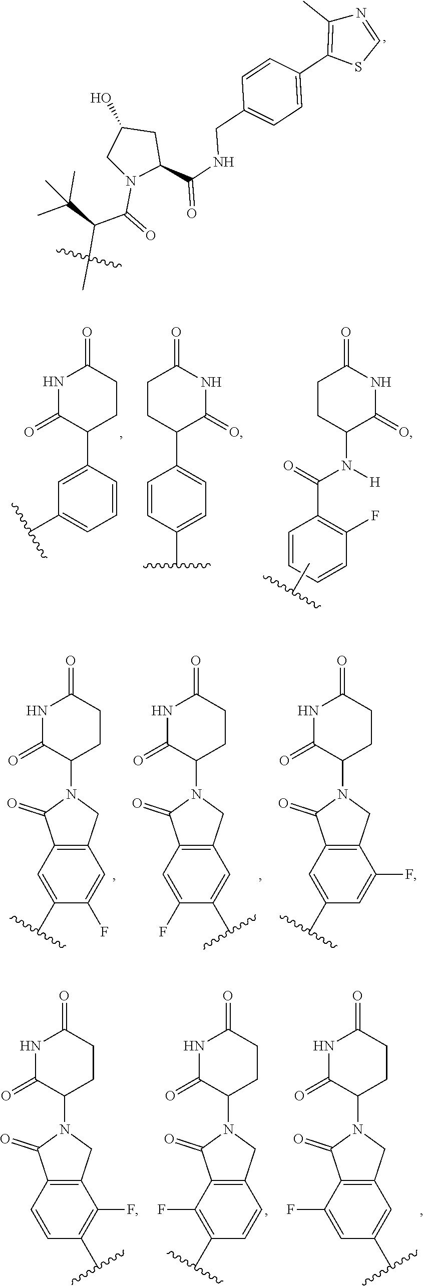

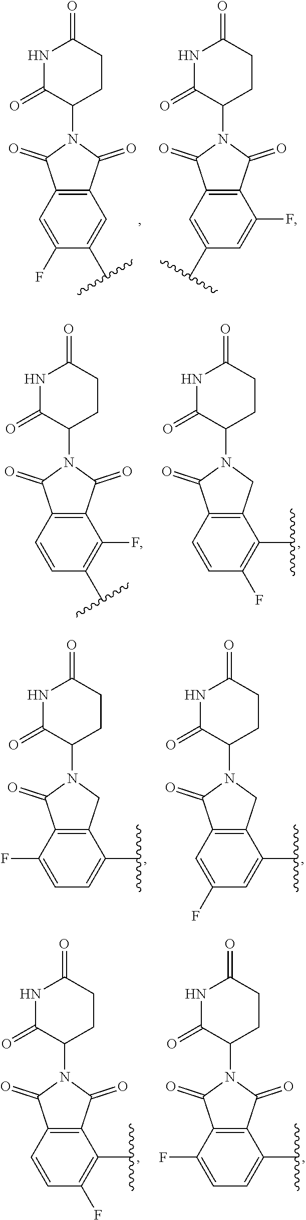

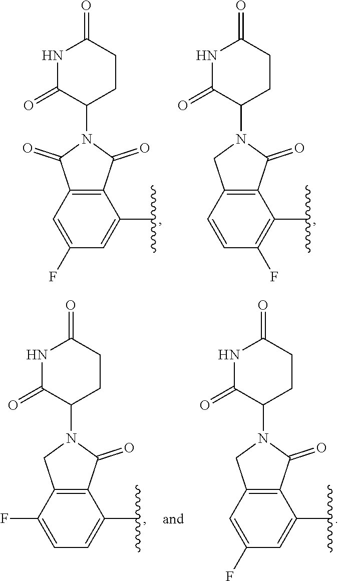

In some embodiments, E is selected from:

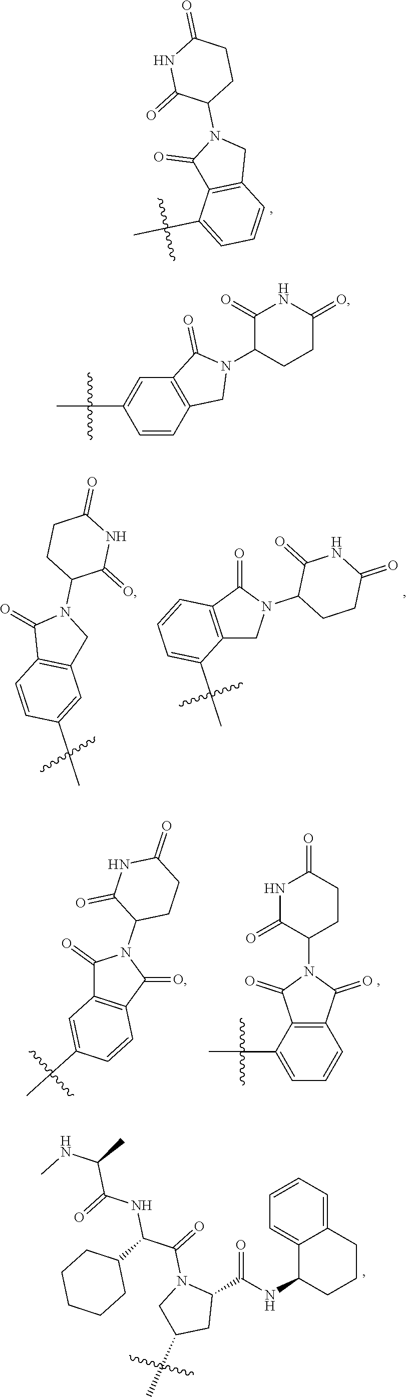

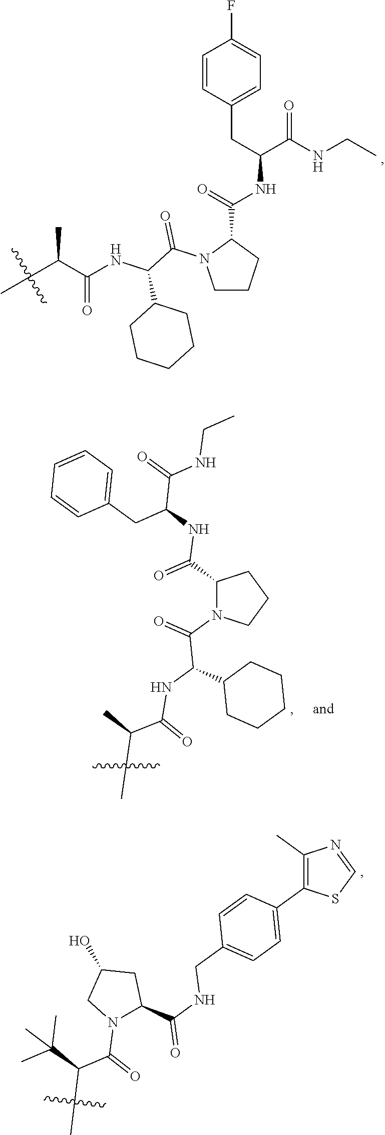

In some embodiments, E is

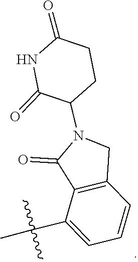 [SMILES: *c1cccc2c1C(=O)N(C1CCC(=O)NC1=O)C2]

In some embodiments, E is

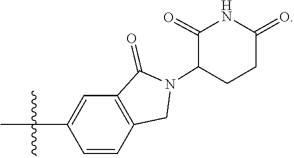 [SMILES: *c1ccc2c(c1)C(=O)N(C1CCC(=O)NC1=O)C2]

In some embodiments, E is

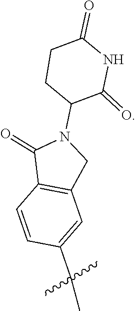 [SMILES: *c1ccc2c(c1)CN(C1CCC(=O)NC1=O)C2=O]

In some embodiments, E is

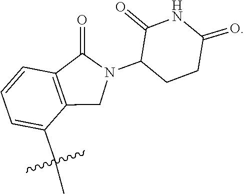 [SMILES: *c1cccc2c1CN(C1CCC(=O)NC1=O)C2=O]

In some embodiments, E is

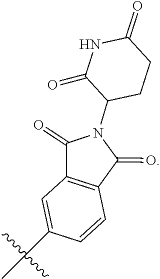 [SMILES: *c1ccc2c(c1)C(=O)N(C1CCC(=O)NC1=O)C2=O]

In some embodiments, E is

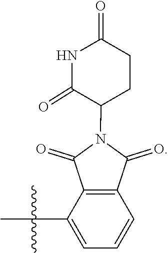 [SMILES: *c1cccc2c1C(=O)N(C1CCC(=O)NC1=O)C2=O]

In some embodiments, E is

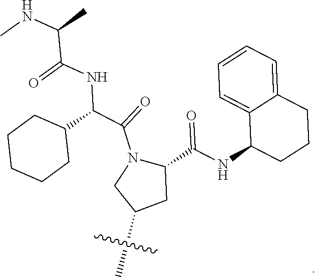

In some embodiments, E is

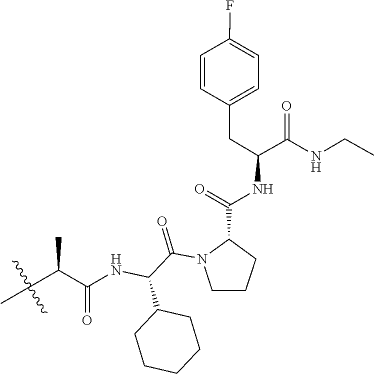 [SMILES: *[C@@H](C)C(=O)N[C@H](C(=O)N1CCC[C@H]1C(=O)N[C@@H](Cc1ccc(F)cc1)C(=O)NCC)C1CCCCC1]

In some embodiments, E is

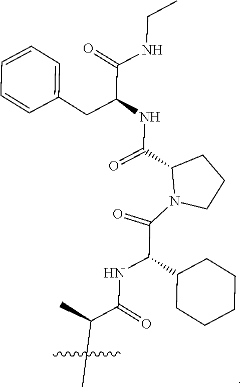 [SMILES: *[C@@H](C)C(=O)N[C@H](C(=O)N1CCC[C@H]1C(=O)N[C@@H](Cc1ccccc1)C(=O)NCC)C1CCCCC1]

In some embodiments, E is

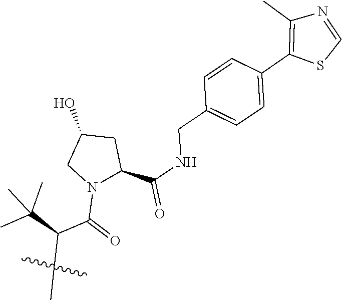 [SMILES: *[C@H](C(=O)N1C[C@H](O)C[C@H]1C(=O)NCc1ccc(-c2scnc2C)cc1)C(C)(C)C]

In some embodiments, the present application provides a compound of Formula I:

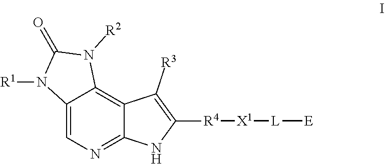

or a pharmaceutically acceptable salt thereof, wherein:
R¹ is selected from H, C₁₋₆ alkyl, C₂₋₆ alkenyl, and C₂₋₆ alkynyl, wherein the C₁₋₆ alkyl, C₂₋₆ alkenyl, and C₂₋₆ alkynyl of R¹ are each optionally substituted with 1, 2, or 3 independently selected R^{1A} substituents;
each R^{1A} is independently selected from halo, oxo, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, R^{c11}R^{d11}, NR^{c11}NR^{c11}R^{d11}, NR^{c11}(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11}, OS(O)(═NR^{e11})R^{b11}, and OS(O)₂R^{b11};
each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{a11}, R^{b11}, R^{c11} and R^{d11} are each optionally substituted with 1, 2, 3, 4, 5, 6, 7, or 8 independently selected R^{M} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, and C₂₋₆ alkynyl;
R² is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{2A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, NR^{c21}R^{d21}, NR^{c21}R^{d21}NR^{c21}C(O)R^{b21}, NR^{c21}C(O)OR^{a21}NR^{c21}C(O)NR^{c21}R^{d21}C(═NR^{e21})R^{b21}, C(═NR^{e21})NR^{c21}R^{d21}, NR^{c21}C(═NR^{e21})NR^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, OS(O)(═NR^{e21})R^{b21}, OS(O)₂R^{b21}, SF₅, P(O)R^{f21}R^{g21}, OP(O)(OR^{h21})(ORⁱ²¹), P(O)(OR^{h21})(ORⁱ²¹), and BR^{j21}R^{k21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f21} and R^{g21} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h21} and Rⁱ²¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a22}, SR^{a22}, NHOR^{a22}, C(O)R^{b22}, C(O)NR^{c22}R^{d22}, C(O)NR^{c22}(OR^{a22}), C(O)OR^{a22}, OC(O)R^{b22}, OC(O)NR^{c22}R^{d22}, NR^{c22}R^{d22}, NR^{c22}NR^{c22}R^{d22}, NR^{c22}C(O)R^{b22}, NR^{c22}C(O)OR^{a22}, NR^{c22}C(O)NR^{c22}R^{d22}C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)R^{b22}, NR^{c22}S(O)NR^{c22}R^{d22}, NR^{c22}S(O)₂R^{b22}, NR^{c22}S(O)(═NR^{e22})R^{b22}, NR^{c22}S(O)₂NR^{c22}R^{d22} S(O)R^{b22}, S(O)NR^{c22}R^{d22}, S(O)₂R^{b22}, S(O)₂NR^{c22}R^{d22}, OS(O)(═NR^{e22})R^{b22}, OS(O)₂R^{b22}, SF₅, P(O)R^{f22}R^{g22}, OP(O)(OR^{h22})(ORⁱ²²), P(O)(OR^{h22})(ORⁱ²²), and BR^{j22}R^{k22}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2B} are each optionally substituted with 1, 2, 3, 4, 5, 6, 7, or 8 independently selected R^{2C} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a22}, R^{c22} and R^{d22} are each optionally substituted with 1, 2, 3, 4, 5, 6, 7, or 8 independently selected R^{2C} substituents;
or, any R^{c22} and R^{d22} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2C} substituents;
each R^{b22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b22} are each optionally substituted with 1, 2, 3, 4, 5, 6, 7, or 8 independently selected R^{2C} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f22} and R^{g22} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h22} and Rⁱ²² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a23}, SR^{a23}, NHOR^{a23}, C(O)R^{b23}, C(O)NR^{c23}R^{d23}, C(O)NR^{c23}(OR^{a23}), C(O)OR^{a23}, OC(O)R^{b23}, OC(O)NR^{c23}R^{d23}, NR^{c23}R^{d23}, NR^{c23}NR^{c23}R^{d23}, NR^{c23}C(O)R^{b23}, NR^{c23}C(O)OR^{a23}, NR^{c23}C(O)NR^{c23}R^{d23}, C(═NR^{e23})R^{b23}, C(═NR^{e23})NR^{c23}R^{d23}, NR^{e23}C(═NR^{e23})NR^{e23}R^{d23}, NR^{c23}C(═NR^{e23})R^{b23}, NR^{c23}S(O)R^{b23}, NR^{c23}S(O)NR^{c23}R^{d23}, NR^{c23}S(O)₂R^{b23}, NR^{c23}S(O)(═NR^{e23})R^{b23}, NR^{c23}S(O)₂NR^{c23}R^{d23}, S(O)R^{b23}, S(O)NR^{e23}R^{d23}, S(O)₂R^{b23}, S(O)₂NR^{c23}R^{d23}, OS(O)(═NR^{e23})R^{b23}, and OS(O)₂R^{b23}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{2C} are each optionally substituted with 1, 2, 3, or 4 independently selected R^{M} substituents;
each R^{a23}, R^{c23}, and R^{d23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a23}, R^{c23} and R^{d23} are each optionally substituted with 1, 2, 3, or 4 independently selected R^{M} substituents;
or, any R^{c23} and R^{d23} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b23} are each optionally substituted with 1, 2, 3, or 4 independently selected R^{M} substituents;
each R^{e23} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R³ is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)NR^{c3}R^{d3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, 4, 5, 6, 7, or 8 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a3}, R^{c3} and R^{d3} are each optionally substituted with 1, 2, 3, 4, 5, 6, 7, or 8 independently selected R^{3A} substituents;
or, any R^{c3} and R^{d3} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3A} substituents;
each R^{b3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b3} are each optionally substituted with 1, 2, 3, 4, 5, 6, 7, or 8 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f3} and R^{g3} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h3} and Rⁱ³ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j3} and R^{k3} is independently selected from OH, C₁₋₆ alkoxy, and C₁₋₆ haloalkoxy;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a31}, SR^{a31}, NHOR^{a31}, C(O)R^{b31}, C(O)NR^{c31}R^{d31}, C(O)NR^{c3}(OR^{a31}), C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}C(O)NR^{c31}R^{d31}, C(═NR^{e31})R^{b31}, C(═NR^{e3})NR^{c31}R^{d31}, NR^{c31}C(═NR^{e31})NR^{c31}R^{d31}, NR^{c31}C(═NR^{e31})R^{b31}, NR^{c31}S(O)R^{b31}, NR^{c31}S(O)NR^{c31}R^{d31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, SF₅, P(O)R^{f31}R^{g31}, OP(O)(OR^{h31})(ORⁱ³¹), P(O)(OR^{h31})(ORⁱ³¹), and BR^{j31}R^{k31}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{3A} are each optionally substituted with 1, 2, 3, 4, 5, 6, 7, or 8 independently selected R^{3B} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a}, R^{c31} and R^{d31} are each optionally substituted with 1, 2, 3, 4, 5, 6, 7, or 8 independently selected R^{3B} substituents;
or, any R^{c31} and R^{d31} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3B} substituents;
each R^{b31} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b31} are each optionally substituted with 1, 2, 3, 4, 5, 6, 7, or 8 independently selected R^{3B} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f31} and R^{g31} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h31} and Rⁱ³¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j31} and R^{k31} is independently selected from OH, C₁₋₆ alkoxy, and C₁₋₆ haloalkoxy;
or any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a32}, SR^{a32}, NHOR^{a32}, C(O)R^{b32}, C(O)NR^{c32}R^{d32}, C(O)NR^{c32}(OR^{a32}), C(O)OR^{a32}, OC(O)R^{b32}, OC(O)NR^{c32}R^{d32}, NR^{c32}R^{d32}, NR^{c32}NR^{c32}R^{d32}, NR^{c32}C(O)R^{b32}, NR^{c32}C(O)OR^{a32}, NR^{c32}C(O)NR^{c32}R^{d32}C(═NR^{e32})R^{b32}, C(═NR^{e32})NR^{c32}R^{d32}, NR^{c32}C(═NR^{e32})R^{c32}R^{d32}, NR^{c32}C(═NR^{e32})R^{b32}, NR^{c32}S(O)R^{b32}, NR^{c32}S(O)NR^{c32}R^{d32}, NR^{c32}S(O)₂R^{b32}, NR^{c32}S(O)(═NR^{e32})R^{b32}, NR^{c32}S(O)₂NR^{c32}R^{d32} S(O)R^{b32}, S(O)NR^{c32}R^{d32}, S(O)₂R^{b32}, S(O)₂NR^{c32}R^{d32}, OS(O)(═NR^{e32})R^{b32}, OS(O)₂R^{b32}, SF₅, P(O)R^{f32}R^{g32}, OP(O)(OR^{h32})(ORⁱ³²), P(O)(OR^{h32})(ORⁱ³²), and BR^{j32}R^{k32}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{3B} are each optionally substituted with 1, 2, 3, 4, 5, 6, 7, or 8 independently selected R^{3C} substituents;
each R^{a32}, R^{c32}, and R^{d32} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a32}, R^{c32} and R^{d32} are each optionally substituted with 1, 2, 3, 4, 5, 6, 7, or 8 independently selected R^{3C} substituents;
or, any R^{c32} and R^{d32} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3C} substituents;
each R^{b32} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b32} are each optionally substituted with 1, 2, 3, 4, 5, 6, 7, or 8 independently selected R^{3C} substituents;
each R^{e32} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f32} and R^{g32} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h32} and Rⁱ³² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j32} and R^{k32} is independently selected from OH, C₁₋₆ alkoxy, and C₁₋₆ haloalkoxy;
or any R^{j32} and R^{k32} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a3}, SR^{a33}, NHOR^{a33}, C(O)R^{b33}, C(O)NR^{c33}R^{d33}, C(O)NR^{c33}(OR^{a33}), C(O)OR^{a33}, OC(O)R^{b33}, OC(O)NR^{c33}R^{d33}, NR^{c33}R^{d33}, NR^{c33}NR^{c33}R^{d33}, NR^{c33}C(O)R^{b33}, NR³³C(O)OR^{a33}, NR^{c33}C(O)NR^{c33}R^{d33}, C(═NR^{e33})R^{b33}, C(═NR^{e33})NR^{c33}R^{d33}, NR^{c33}C(═NR^{e33})NR^{c33}R^{d33}, NR^{c33}C(═NR^{e33})R^{b33}, NR^{c33}S(O)R^{b33}, NR^{c33}S(O)NR^{c33}R^{d33}, NR^{c33}S(O)₂R^{b33}, NR^{c33}S(O)(═NR^{e33})R^{b33}, NR^{c33}S(O)₂NR^{c33}R^{d33}, S(O)R^{b33}, S(O)NR^{c33}R^{d33}, S(O)₂R^{b33}, S(O)₂NR^{c33}R^{d33}, OS(O)(═NR^{e33})R^{b33}, and OS(O)₂R^{b33}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{3C} are each optionally substituted with 1, 2, 3, or 4 independently selected R^{M} substituents;
each R^{a33}, R^{c33}, and R^{d33} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a33}, R^{c33} and R^{d33} are each optionally substituted with 1, 2, 3, or 4 independently selected R^{M} substituents;
or, any R^{c33} and R^{d33} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b33} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b33} are each optionally substituted with 1, 2, 3, or 4 independently selected R^{M} substituents;
each R^{e33} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from C₁₋₆ alkylene, C₁₋₆ haloalkylene, C₂₋₆ alkenylene, C₂₋₆ alkynylene, C₆₋₁₀ arylene, C₃₋₁₀ cycloalkylene, 5-10 membered heteroarylene, 4-10 membered heterocycloalkylene, —C₆₋₁₀ arylene-C₁₋₆ alkylene-, —C₃₋₁₀ cycloalkylene-C₁₋₆ alkylene-, -(5-10 membered heteroarylene)-C₁₋₆ alkylene-, and -(4-10 membered heterocycloalkylene)-C₁₋₆ alkylene-, wherein the C₁₋₆ alkylene, C₂₋₆ alkenylene, C₂₋₆ alkynylene, C₆₋₁₀ arylene, C₃₋₁₀ cycloalkylene, 5-10 membered heteroarylene, 4-10 membered heterocycloalkylene, —C₆₋₁₀ aryl-C₁₋₆ alkylene-, —C₃₋₁₀ cycloalkyl-C₁₋₆ alkylene-, -(5-10 membered heteroaryl)-C₁₋₆ alkylene-, and -(4-10 membered heterocycloalkyl)-C₁₋₆ alkylene- of R⁴ are each optionally substituted with 1, 2, 3, 4, 5, 6, 7, or 8 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a41}, SR^{a41}, NHOR^{a41}, C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41}, OS(O)(═NR^{e41})R^{b41}, OS(O)₂R^{b41}, SF₅, P(O)R^{f41}R^{g41}, OP(O)(OR^{h41})(ORⁱ⁴¹), P(O)(OR^{h41})(ORⁱ⁴¹), and BR^{j41}R^{k41}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{4A} are each optionally substituted with 1, 2, 3, 4, 5, 6, 7, or 8 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a41}, R^{c41} and R^{d41} are each optionally substituted with 1, 2, 3, 4, 5, 6, 7, or 8 independently selected R^{4B} substituents;
or, any R^{c41} and R^{d41} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4B} substituents;
each R^{b41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b41} are each optionally substituted with 1, 2, 3, 4, 5, 6, 7, or 8 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f41} and R^{g41} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h41} and Rⁱ⁴¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j41} and R^{k41} is independently selected from OH, C₁₋₆ alkoxy, and C₁₋₆ haloalkoxy;
or any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a42}, SR^{a42}, NHOR^{a42}, C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)R^{b42}, NR^{c42}S(O)NR^{c42}R^{d42}, NR^{c42}S(O)₂R^{b42}, NR^{c42}S(O)(═NR^{e42})R^{b42}, NR^{c42}S(O)₂NR^{c42}R^{d42} S(O)R^{b42}, S(O)NR^{c42}R^{d42}, S(O)₂R^{b42}, S(O)₂NR^{c42}R^{d42}, OS(O)(═NR^{e42})R^{b42}, OS(O)₂R^{b42}, SF₅, P(O)R^{f42}R^{g42}, OP(O)(OR^{h42})(ORⁱ⁴²), P(O)(OR^{h42})(ORⁱ⁴²), and BR^{j42}R^{k42}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{4B} are each optionally substituted with 1, 2, 3, 4, 5, 6, 7, or 8 independently selected R^{4C} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a42}, R^{c42} and R^{d42} are each optionally substituted with 1, 2, 3, 4, 5, 6, 7, or 8 independently selected R^{4C} substituents;
or, any R^{c42} and R^{d42} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4C} substituents;
each R^{b42} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b42} are each optionally substituted with 1, 2, 3, 4, 5, 6, 7, or 8 independently selected R^{4C} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f42} and R^{g42} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h42} and Rⁱ⁴² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j42} and R^{k42} is independently selected from OH, C₁₋₆ alkoxy, and C₁₋₆ haloalkoxy;
or any R^{j42} and R^{k42} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a43} SR^{a43}, NHOR^{a43}, C(O)R^{b43}, C(O)NR^{c43}R^{d43}, C(O)NR^{c43}(OR^{a43}), C(O)OR^{a43}, OC(O)R^{b43}, OC(O)NR^{c43}R^{d43}, NR^{c43}R^{d43}, NR^{c43}NR^{c43}R^{d43}, NR^{c43}C(O)R^{b43}, NR^{c43}C(O)OR^{a43}, NR^{c43}C(O)NR^{c43}R^{d43}, C(═NR^{e43})R^{b43}, C(═NR^{e43})NR^{c43}R^{d43}, NR^{c43}C(═NR^{e43})NR^{c43}R^{d43}, NR^{c43}C(═NR^{e43})R^{b43}, NR^{c43}S(O)R^{b43}, NR^{c43}S(O)NR^{c43}R^{d43}, NR^{c43}S(O)₂R^{b43}, NR^{c43}S(O)(═NR^{e43})R^{b43}, NR^{c43}S(O)₂NR^{c43}R^{d43}, S(O)R^{b43}, S(O)NR^{c43}R^{d43}, S(O)₂R^{b43}, S(O)₂NR^{c43}R^{d43}, OS(O)(═NR^{e43})R^{b43}, and OS(O)₂R^{b43}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{4C} are each optionally substituted with 1, 2, 3, or 4 independently selected R^{M} substituents;
each R^{a43}, R^{c43}, and R^{d43} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a43}, R^{c43} and R^{d43} are each optionally substituted with 1, 2, 3, or 4 independently selected R^{M} substituents;
or, any R^{c43} and R^{d43} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b43} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b43} are each optionally substituted with 1, 2, 3, or 4 independently selected R^{M} substituents;
each R^{e43} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
X¹ is selected from —CH₂—, C(O), and —CH₂C(O)—;

L is selected from —N(R^{N1})—R⁵—N(R^{N2})—, —N(R^{N1})—R⁵—O—,

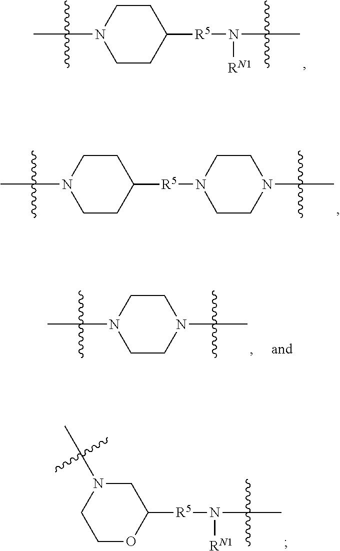

R^{N1} is H or C₁₋₆ alkyl;
R^{N2} is H or C₁₋₆ alkyl;
R⁵ is selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ haloalkylene-O—C₁₋₆ alkylene-C(O)—, —C₁₋₆ alkylene-C₃-7 cycloalkylene-C(O)—, and —C₁₋₆ alkylene-phenyl-C(O)—;
E is selected from:

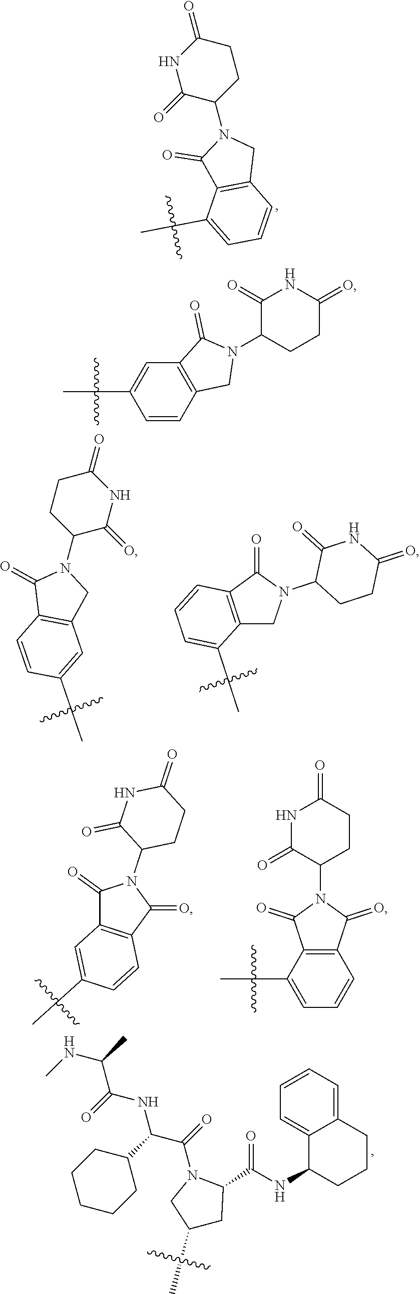

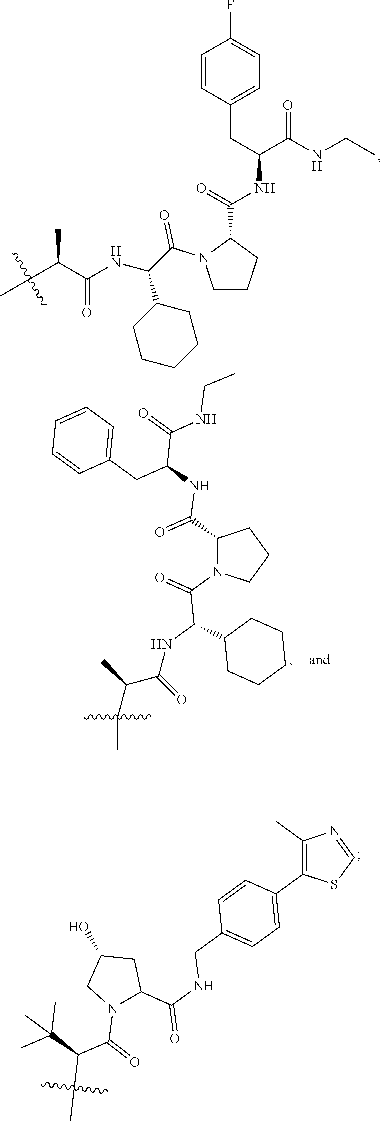

and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

In some embodiments:
R¹ is selected from H, C₁₋₆ alkyl, C₂₋₆ alkenyl, and C₂₋₆ alkynyl, wherein the C₁₋₆ alkyl, C₂₋₆ alkenyl, and C₂₋₆ alkynyl;
R² is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{2A} is independently selected from NR^{c21}C(O)R^{b21}, NR^{c21}C(O)OR^{a21}, and NR^{c21}C(O)NR^{c21}R^{d21};
each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl;
R³ is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, or 4 independently selected R^{3A} substituents;
each R^{3A} is independently selected from halo, oxo, C₁₋₆ alkyl, C₁₋₆ haloalkyl, C₂₋₆ alkenyl, C₂₋₆ alkynyl, and OR^{a31};
each R^{a31} is independently selected from H, C₁₋₆ alkyl, C₁₋₆ haloalkyl, C₂₋₆ alkenyl, and C₂₋₆ alkynyl;
R⁴ is selected from C₆₋₁₀ arylene, C₃₋₁₀ cycloalkylene, 5-10 membered heteroarylene, and 4-10 membered heterocycloalkylene, wherein the C₆₋₁₀ arylene, C₃₋₁₀ cycloalkylene, 5-10 membered heteroarylene, and 4-10 membered heterocycloalkylene of R⁴ are each optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, and OR^{a41};
each R^{a41} is independently selected from H, C₁₋₆ alkyl, C₁₋₆ haloalkyl, C₂₋₆ alkenyl, and C₂₋₆ alkynyl;
X¹ is selected from —CH₂—, C(O), and —CH₂C(O)—;
L is selected from —N(R^{N1})—R⁵—N(R^{N2})—, —N(R^{N1})—R⁵—O—,

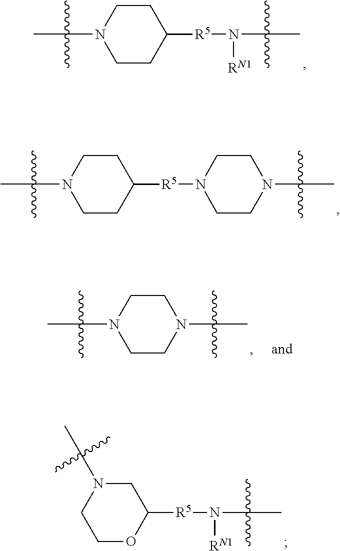

R^{N1} is H or C₁₋₃ alkyl;
R^{N2} is H or C₁₋₃ alkyl;
R⁵ is selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ haloalkylene-O—C₁₋₆ alkylene-C(O)—, —C₁₋₆ alkylene-C₃-7 cycloalkylene-C(O)—, and —C₁₋₆ alkylene-phenyl-C(O)—; and
E is selected from:

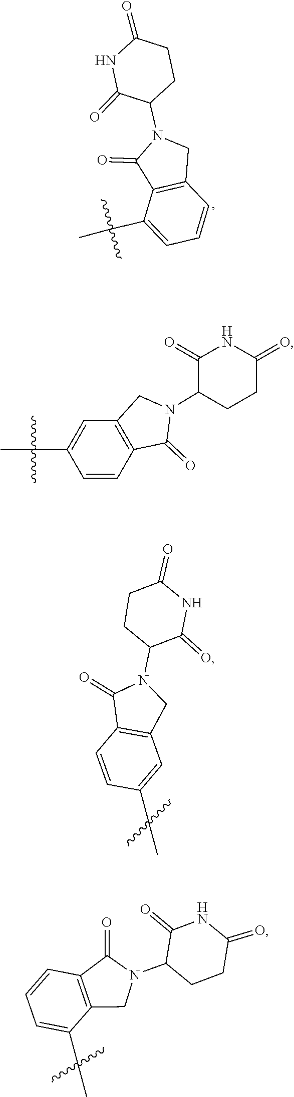 [SMILES: *c1cccc2c1C(=O)N(C1CCC(=O)NC1=O)C2 ; *c1ccc2c(c1)CN(C1CCC(=O)NC1=O)C2=O ; *c1ccc2c(c1)CN(C1CCC(=O)NC1=O)C2=O ; *c1cccc2c1CN(C1CCC(=O)NC1=O)C2=O]

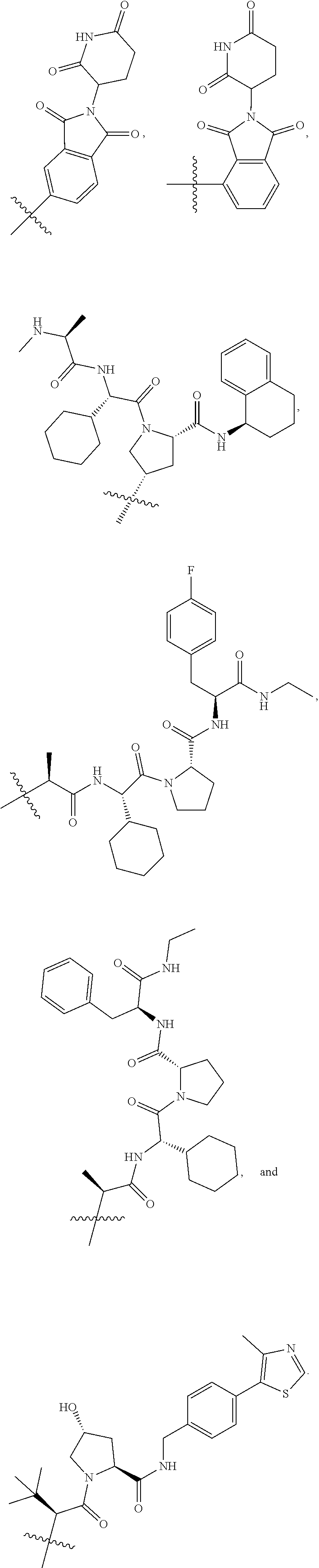

In some embodiments:
R¹ is C₁₋₆ alkyl;
R² is C₃₋₇ cycloalkyl, which is optionally substituted with 1, 2, 3, or 4 independently selected R^{2A} substituents;
each R^{2A} is independently selected from NR^{c21}C(O)OR^{a21};
each R^{a21} and R^{c21} is independently selected from H and C₁₋₆ alkyl;
R³ is selected from phenyl, 8-10 membered heteroaryl, and 8-10 membered heterocycloalkyl, wherein the phenyl, 8-10 membered heteroaryl, and 8-10 membered heterocycloalkyl of R³ are each optionally substituted with 1, 2, 3, or 4 independently selected R^{3A} substituents;
each R^{3A} is independently selected from halo, C₁₋₆ alkyl, C₁₋₆ haloalkyl, and OR^{a31};
each R^{a31} is independently selected from H and C₁₋₆ alkyl;
R⁴ is selected from phenylene and 5-6 membered heteroarylene, wherein the phenylene and 5-6 membered heteroarylene of R⁴ are each optionally substituted with 1, 2, 3, or 4 independently selected R^{4A} substituents;
each R^{4A} is independently selected from halo and OR^{a41};
each R^{a31} is independently selected from H and C₁₋₆ alkyl;
X¹ is selected from —CH₂—, C(O), and —CH₂C(O)—;
L is selected from —N(R^{N1})—R⁵—N(R^{N2})—, —N(R^{N1})—R⁵—O—,

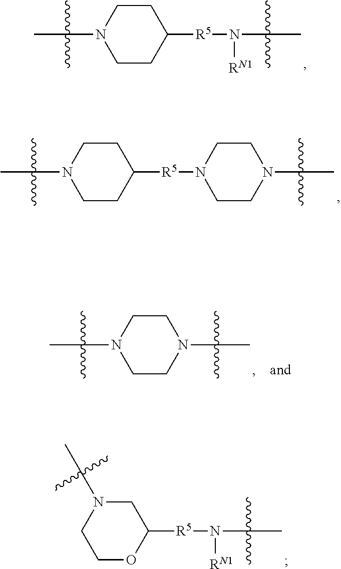

R^{N1} is H or C₁₋₃ alkyl;
R^{N2} is H or C₁₋₃ alkyl;
R⁵ is selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ haloalkylene-O—C₁₋₆ alkylene-C(O)—, —C₁₋₆ alkylene-C₃₋₇ cycloalkylene-C(O)—, and —C₁₋₆ alkylene-phenyl-C(O)—; and
E is selected from:

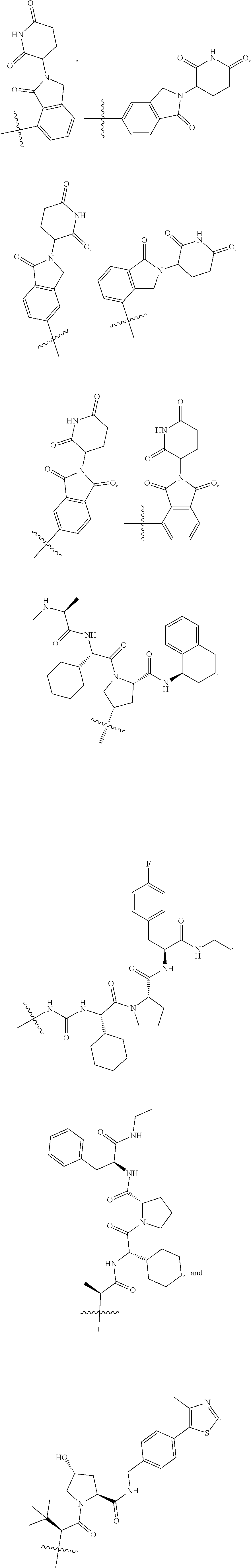

In some embodiments:
R¹ is C₁₋₃ alkyl;
R² is C₃₋₇ cycloalkyl, which is optionally substituted with 1, 2, 3, or 4 independently selected R^{2A} substituents;
each R^{2A} is independently selected from C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21}, R^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, NR^{c21}S(O)R^{b21}, NR^{c21}S(O)NR^{c21}R^{d21}, NR^{c21}S(O)₂R^{b21}, NR^{c21}S(O)₂NR^{c21}R^{d21}, S(O)R^{b21}, S(O)NR^{c21}R^{d21}, S(O)₂R^{b21}, S(O)₂NR^{c21}R^{d21}, and OS(O)₂R^{b21};
each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl;
R³ is selected from phenyl, indazolyl, and dihydroisobenzofuranyl, wherein the phenyl, indazolyl, and dihydroisobenzofuranyl of R³ are each optionally substituted with 1, 2, 3, or 4 independently selected R^{3A} substituents;
each R^{3A} is independently selected from halo, C₁₋₆ alkyl, C₁₋₆ haloalkyl, and OR^{a31};
each R^{a31} is independently selected from H, C₁₋₆ alkyl, C₁₋₆ haloalkyl, C₂₋₆ alkenyl, and C₂₋₆ alkynyl.

In some embodiments:
R¹ is C₁₋₃ alkyl;
R² is C₃₋₇ cycloalkyl, which is optionally substituted with 1, 2, 3, or 4 independently selected R^{2A} substituents;
each R^{2A} is independently selected from NR^{c21}C(O)R^{b21}, NR^{c21}C(O)OR^{a21}, and NR^{c21}C(O)NR^{c21}R^{d21};
each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl;
R³ is selected from phenyl, indazolyl, and dihydroisobenzofuranyl, wherein the phenyl, indazolyl, and dihydroisobenzofuranyl of R³ are each optionally substituted with 1, 2, 3, or 4 independently selected R^{3A} substituents;
each R^{3A} is independently selected from halo, C₁₋₆ alkyl, C₁₋₆ haloalkyl, and OR^{a31};
each R^{a31} is independently selected from H, C₁₋₆ alkyl, C₁₋₆ haloalkyl, C₂₋₆ alkenyl, and C₂₋₆ alkynyl.

In some embodiments:
R¹ is C₁₋₃ alkyl;
R² is C₃₋₇ cycloalkyl, which is optionally substituted with 1 R^{2A} substituent;
R^{2A} is NR^{c21}C(O)OR^{a21};
R^{a21} and R^{c21} are each independently selected from H and C₁₋₃ alkyl;
R³ is selected from phenyl, indazolyl, and dihydroisobenzofuranyl, wherein the phenyl, indazolyl, and dihydroisobenzofuranyl of R³ are each optionally substituted with 1, 2, 3, or 4 independently selected R^{3A} substituents;
each R^{3A} is independently selected from halo, C₁₋₆ alkyl, C₁₋₆ haloalkyl, and OR^{a31};
each R^{a31} is independently selected from H and C₁₋₃ alkyl.

In some embodiments, the compound of Formula I is a compound of Formula II:

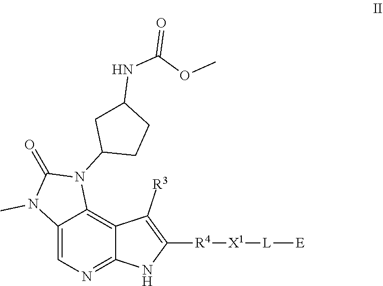

or a pharmaceutically acceptable salt thereof.

In some embodiments, the compound of Formula I is a compound of Formula III:

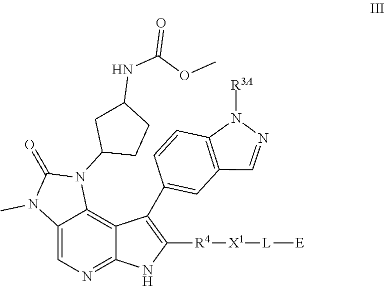

or a pharmaceutically acceptable salt thereof.

In some embodiments, the compound of Formula I is a compound of Formula IV:

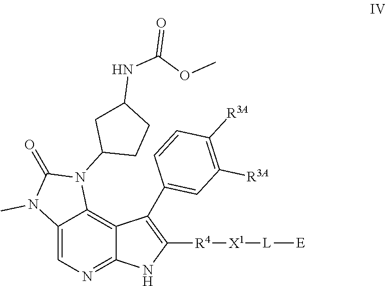

or a pharmaceutically acceptable salt thereof.

In some embodiments, the compound of Formula I is a compound of Formula IV:

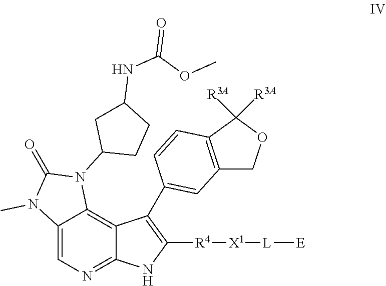

or a pharmaceutically acceptable salt thereof.

In some embodiments, the compound provided herein is selected from:
methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((3-((2-(2,6-dioxopiperidin-3-yl)-1,3-dioxoisoindolin-5-yl)amino)propyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((2-(2,6-dioxopiperidin-3-yl)-1,3-dioxoisoindolin-5-yl)amino)butyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((5-((2-(2,6-dioxopiperidin-3-yl)-1,3-dioxoisoindolin-5-yl)amino)pentyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((4-((2-(2,6-dioxopiperidin-3-yl)-1,3-dioxoisoindolin-5-yl)amino)butyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1,3-dioxoisoindolin-4-yl)amino)-2-oxoethoxy)ethyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((3-((2-(2,6-dioxopiperidin-3-yl)-1,3-dioxoisoindolin-5-yl)amino)propyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((5-((2-(2,6-dioxopiperidin-3-yl)-1,3-dioxoisoindolin-5-yl)amino)pentyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((2-(2-((2-(2,6-dioxopiperidin-3-yl)-1-oxoisoindolin-5-yl)amino)-2-oxoethoxy)ethyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((2-(2,6-dioxopiperidin-3-yl)-1,3-dioxoisoindolin-5-yl)amino)butyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((5-((2-(2,6-dioxopiperidin-3-yl)-1,3-dioxoisoindolin-5-yl)amino)pentyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(3-fluoro-4-((2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(3-fluoro-4-((2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-(3-((2-(2,6-dioxopiperidin-3-yl)-1-oxoisoindolin-5-yl)amino)-3-oxopropoxy)ethyl)piperazine-1-carbonyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-((2-(2,6-dioxopiperidin-3-yl)-1-oxoisoindolin-5-yl)carbamoyl)benzyl)piperazine-1-carbonyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-((2-(2,6-dioxopiperidin-3-yl)-1-oxoisoindolin-5-yl)carbamoyl)benzyl)piperazine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((7-((2-(2,6-dioxopiperidin-3-yl)-1-oxoisoindolin-5-yl)amino)-7-oxoheptyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-((2-(2,6-dioxopiperidin-3-yl)-1,3-dioxoisoindolin-4-yl)carbamoyl)benzyl)piperazine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1,3-dioxoisoindolin-4-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-(3-(2-((2-(2,6-dioxopiperidin-3-yl)-1,3-dioxoisoindolin-4-yl)amino)-2-oxoethoxy)-2,2-difluoropropoxy)butyl)piperazine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-3-oxoisoindolin-4-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-4-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-3-oxoisoindolin-5-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1,3-dioxoisoindolin-5-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)(methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(((S)-1-(((S)-1-cyclohexyl-2-((S)-2-(((S)-1-(ethylamino)-1-oxo-3-phenylpropan-2-yl)carbamoyl)pyrrolidin-1-yl)-2-oxoethyl)amino)-1-oxopropan-2-yl)amino)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((6-(((S)-1-(((S)-1-cyclohexyl-2-((S)-2-(((S)-1-(ethylamino)-1-oxo-3-phenylpropan-2-yl)carbamoyl)pyrrolidin-1-yl)-2-oxoethyl)amino)-1-oxopropan-2-yl)amino)hexyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(((S)-1-(((S)-1-cyclohexyl-2-((S)-2-(((S)-1-(ethylamino)-3-(4-fluorophenyl)-1-oxopropan-2-yl)carbamoyl)pyrrolidin-1-yl)-2-oxoethyl)amino)-1-oxopropan-2-yl)amino)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-5-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-5-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1-oxoisoindolin-5-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1-oxoisoindolin-5-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-(2-(2,6-dioxopiperidin-3-yl)-1,3-dioxoisoindolin-4-yl)piperazine-1-carbonyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((1-(2-(2,6-dioxopiperidin-3-yl)-1,3-dioxoisoindolin-4-yl)piperidin-4-yl)methyl)piperazine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((2-(2,6-dioxopiperidin-3-yl)-1,3-dioxoisoindolin-4-yl)amino)butyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(1-(2-(4-((4-(2-(2,6-dioxopiperidin-3-yl)-1,3-dioxoisoindolin-4-yl)piperazin-1-yl)methyl)piperidin-1-yl)-2-oxoethyl)-1H-pyrazol-4-yl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(1-(2-(4-((4-(2-(2,6-dioxopiperidin-3-yl)-1-oxoisoindolin-5-yl)piperazin-1-yl)methyl)piperidin-1-yl)-2-oxoethyl)-1H-pyrazol-4-yl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((1-(2-(2,6-dioxopiperidin-3-yl)-1,3-dioxoisoindolin-4-yl)piperidin-4-yl)methyl)piperazin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((4-(2-(2,6-dioxopiperidin-3-yl)-1-oxoisoindolin-5-yl)piperazin-1-yl)methyl)piperidin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((4-(2-(2,6-dioxopiperidin-3-yl)-1,3-dioxoisoindolin-4-yl)piperazin-1-yl)methyl)piperidin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans)-4-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)carbamoyl)cyclohexyl)methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans-)-4-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)carbamoyl)cyclohexyl)methyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(3-((2-(2,6-dioxopiperidin-3-yl)-1-oxoisoindolin-5-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-((2-(2,6-dioxopiperidin-3-yl)-1-oxoisoindolin-5-yl)amino)-2-oxoethyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans-)-4-((2-(2,6-dioxopiperidin-3-yl)-1-oxoisoindolin-5-yl)carbamoyl)cyclohexyl)methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans)-4-((2-(2,6-dioxopiperidin-3-yl)-1-oxoisoindolin-5-yl)carbamoyl)cyclohexyl)methyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(2-((2-(2,6-dioxopiperidin-3-yl)-1-oxoisoindolin-5-yl)carbamoyl)morpholine-4-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((2-(2,6-dioxopiperidin-3-yl)-1-oxoisoindolin-5-yl)carbamoyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((2-(2,6-dioxopiperidin-3-yl)-1-oxoisoindolin-5-yl)carbamoyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate; methyl((1R,3R)-3-(7-(4-(4-(2-((2-(2,6-dioxopiperidin-3-yl)-1-oxoisoindolin-5-yl)amino)-2-oxoethyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-(2,6-dioxopiperidin-3-yl)-1,3-dioxoisoindolin-5-yl)piperazine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate; and methyl((1R,3R)-3-(7-(4-(4-((4-(2-(2,6-dioxopiperidin-3-yl)-3-oxoisoindolin-4-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
   or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 5 to 10 carbon atoms. In some embodiments, the aryl group is phenyl or naphthyl. In some embodiments, the aryl is phenyl.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, “Cₙ₋ₘ haloalkoxy” refers to a group of formula —O-haloalkyl having n to m carbon atoms. Example haloalkoxy groups include OCF₃ and OCHF₂. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “carbonyl”, employed alone or in combination with other terms, refers to a —C(O)— group.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carboxy” refers to a group of formula —C(O)OH.

As used herein, the term “di(Cₙ₋ₘ alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl.

In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, and the like. In some embodiments, cycloalkyl is cyclopropyl, cyclobutyl, cyclopentyl, or cyclohexyl.

As used herein, “heteroaryl” refers to a monocyclic or polycyclic (e.g., having 2 fused rings) aromatic heterocycle having at least one heteroatom ring member selected from N, O, S and B. In some embodiments, the heteroaryl ring has 1, 2, 3, or 4 heteroatom ring members independently selected from N, O, S and B. In some embodiments, any ring-forming N in a heteroaryl moiety can be an N-oxide. In some embodiments, the heteroaryl is a 5-10 membered monocyclic or bicyclic heteroaryl having 1, 2, 3, or 4 heteroatom ring members independently selected from N, O, S, and B. In some embodiments, the heteroaryl is a 5-10 membered monocyclic or bicyclic heteroaryl having 1, 2, 3, or 4 heteroatom ring members independently selected from N, O, and S. In some embodiments, the heteroaryl is a 5-6 monocyclic heteroaryl having 1 or 2 heteroatom ring members independently selected from N, O, S, and B. In some embodiments, the heteroaryl is a 5-6 monocyclic heteroaryl having 1 or 2 heteroatom ring members independently selected from N, O, and S. In some embodiments, the heteroaryl group contains 3 to 10, 4 to 10, 5 to 10, 5 to 7, 3 to 7, or 5 to 6 ring-forming atoms. In some embodiments, the heteroaryl group has 1 to 4 ring-forming heteroatoms, 1 to 3 ring-forming heteroatoms, 1 to 2 ring-forming heteroatoms or 1 ring-forming heteroatom. When the heteroaryl group contains more than one heteroatom ring member, the heteroatoms may be the same or different. Example heteroaryl groups include, but are not limited to, thienyl (or thiophenyl), furyl (or furanyl), pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, 1,3,4-oxadiazolyl and 1,2-dihydro-1,2-azaborine, pyridinyl, pyrimidinyl, pyrazinyl, pyridazinyl, azolyl, triazolyl, thiadiazolyl, quinolinyl, isoquinolinyl, indolyl, benzothiophenyl, benzofuranyl, benzisoxazolyl, imidazo[1,2-b]thiazolyl, purinyl, triazinyl, thieno[3,2-b]pyridinyl, imidazo[1,2-a]pyridinyl, 1,5-naphthyridinyl, 1H-pyrazolo[4,3-b]pyridinyl, triazolo[4,3-a]pyridinyl, 1H-pyrrolo[3,2-b]pyridinyl, 1H-pyrrolo[2,3-b]pyridinyl, pyrazolo[1,5-a]pyridinyl, pyrazolo[1,5-a]pyrimidinyl, indazolyl, and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, tetrahydropyridinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[3.5]nonanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, dihydroisobenzofuranyl, and the like.

As used herein, “Cₒ₋ₚ cycloalkyl-Cₙ₋ₘ alkyl-” refers to a group of formula cycloalkyl-alkylene-, wherein the cycloalkyl has o to p carbon atoms and the alkylene linking group has n to m carbon atoms.

As used herein “Cₒ₋ₚ aryl-Cₙ₋ₘ alkyl-” refers to a group of formula aryl-alkylene-, wherein the aryl has o to p carbon atoms and the alkylene linking group has n to m carbon atoms.

As used herein, “heteroaryl-Cₙ₋ₘ alkyl-” refers to a group of formula heteroaryl-alkylene-, wherein alkylene linking group has n to m carbon atoms.

As used herein “heterocycloalkyl-Cₙ₋ₘ alkyl-” refers to a group of formula heterocycloalkyl-alkylene-, wherein alkylene linking group has n to m carbon atoms.

As used herein, an “alkylene group” is a bivalent straight chain or branched alkyl linking group. For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-diyl, propan-1,2-diyl, propan-1,1-diyl and the like.

As used herein, an “Cₙ₋ₘ alkenylene” group is a bivalent straight chain or branched alkenyl linking group, wherein the alkenyl linking group has n to m carbon atoms. It is understood that the “alkenyl” portion of the alkenyl linking group is defined according to the definition of “alkenyl” provided herein.

As used herein, an “Cₙ₋ₘ alkynylene” group is a bivalent straight chain or branched alkynyl linking group, wherein the alkynyl linking group has n to m carbon atoms. It is understood that the “alkynyl” portion of the alkynyl linking group is defined according to the definition of “alkynyl” provided herein.

As used herein, an “Cₙ₋ₘ alkoxylene” group is a bivalent straight chain or branched alkoxy linking group, wherein the alkoxy linking group has n to m carbon atoms. It is understood that the “alkoxy” portion of the alkoxy linking group is defined according to the definition of “alkoxy” provided herein. Examples of “alkoxylene” or “alkoxy linking groups” include, but are not limited to, —OCH₂—, —OCH₂CH₂—, —OCH(CH₃)CH₂—, —OCH(CH₂CH₃)CH₂—, —OCH₂CH₂CH₂—, —OCH₂CH₂CH₂CH₂—, —OCH₂CH₂CH₂CH₂CH₂—, —OCH₂CH₂CH₂CH₂CH₂CH₂—, and the like.

As used herein, a “Cₙ₋ₘ arylene” group is a bivalent aryl linking group, wherein the aryl linking group has n to m carbon atoms. It is understood that the “aryl” portion of the aryl linking group is defined according to the definition of “aryl” provided herein. Examples of “arylene” or “aryl linking groups” include, but are not limited to, phen-1,2-diyl, phen-1,3-diyl, phen-1,4-diyl, and the like.

As used herein, a “Cₙ₋ₘ cycloalkylene” group is a bivalent cycloalkylene linking group, wherein the cycloalkylene linking group has n to m carbon atoms. It is understood that the “cycloalkyl” portion of the cycloalkyl linking group is defined according to the definition of “cycloalkyl” provided herein. Examples of “cycloalkylene” or “cycloalkyl linking groups” include, but are not limited to, cycloprop-1,2-diyl, cyclobut-1,2-diyl, cyclobut-1,3-diyl, cyclopent-1,2-diyl, cyclopent-1,3-diyl, cyclohex-1,2-diyl, cyclohex-1,3-diyl, cyclohex-1,4-diyl, and the like.

As used herein, a “heterocycloalkylene” group is a bivalent heterocycloalkyl linking group. It is understood that the “heterocycloalkyl” portion of the heterocycloalkyl linking group is defined according to the definition of “heterocycloalkyl” provided herein. Examples of “heterocycloalkylene” or “heterocycloalkyl linking groups” include, but are not limited to, pyran-1,2-diyl, pyran-1,3-diyl, pyran-1,4-diyl, tetrahydropyran-2,3-diyl, tetrahydropyran-2,4-diyl, tetrahydropyran-2,5-diyl, tetrahydropyran-2,6-diyl, tetrahydrofuran-2,3-diyl, tetrahydrofuran-2,4-diyl, tetrahydrofuran-3,4-diyl, tetrahydrofuran-2,5-diyl, piperidin-1,2-diyl, piperidin-1,3-diyl, piperidin-1,4-diyl, piperidin-2,3-diyl, piperidin-2,4-diyl, piperidin-2,5-diyl, piperidin-2,6-diyl, and the like.

As used herein, a “heteroarylene” group is a bivalent heteroaryl linking group. It is understood that the “heteroaryl” portion of the heteroaryl linking group is defined according to the definition of “heteroaryl” provided herein. Examples of “heteroarylene” or “heteroaryl linking groups” include, but are not limited to, pyridin-2,3-diyl, pyridin-2,4-diyl, pyridin-2,5-diyl, pyridin-2,6-diyl, pyridin-3,5-diyl, pyridin-3,4-diyl, furan-2,3-diyl, furan-2,4-diyl, furan-3,4-diyl, and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula Ia, etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula 1-10 can be prepared as shown in Scheme 1. The 3-position of 4-chloro-5-nitro-1-(phenylsulfonyl)-1H-pyrrolo[2,3-b]pyridine 1-1 can be brominated followed by an S_{N}Ar displacement in the presence of base to afford the diamine 1-3. Reduction of the nitro group affords a triamine that can undergo intramolecular cyclization in the presence of CDI to afford the tricyclic lactam 1-5. Methylation of the lactam N—H can occur upon treatment of 1-5 with a strong base, such as an inorganic base, in the presence of a methylating agent, such as iodomethane. Removal of the Boc protecting group and acylation of the resultant primary amine affords carbamate 1-7. Compound 1-7 can be coupled with R³-M¹, where M¹ is a boronic acid, boronate ester, potassium trifluoroborate, or an appropriately substituted metal such as Sn(Bu)₃ or Zn, under standard metal catalyzed coupling conditions, such as Suzuki-Miyaura (e.g., in the presence of a palladium catalyst, such as tetrakis(triphenylphosphine)palladium(O) and a base (e.g., a carbonate base)) or standard Stille conditions (e.g., in the presence of a palladium(O) catalyst, such as tetrakis(triphenylphosphine)palladium(O)) or standard Negishi conditions (e.g., in the presence of a palladium catalyst, such as tetrakis(triphenylphosphine)palladium(O) or [1,1′-bis(diphenylphosphino)ferrocene]dichloropalladium (II)), to give compound 1-8. Bromination can be accomplished via a lithiation reaction with a base such as LDA followed by trapping with an electrophilic halogen source, such as 1,2-dibromotetrachloroethane to afford 1-9. Compound 1-9 can be coupled with R⁴-M², where M² is a boronic acid, boronate ester, potassium trifluoroborate, or an appropriately substituted metal such as Sn(Bu)₃ or Zn, under standard Suzuki-Miyaura conditions (e.g., in the presence of a palladium catalyst, such as tetrakis(triphenylphosphine)palladium(O) and a base (e.g., a carbonate base)) or standard Stille conditions (e.g., in the presence of a palladium(O) catalyst, such as tetrakis(triphenylphosphine)palladium(O)) or standard Negishi conditions (e.g., in the presence of a palladium catalyst, such as tetrakis(triphenylphosphine)palladium(O) or [1,1′-bis(diphenylphosphino)ferrocene]dichloropalladium (II)), to give compound 1-10.

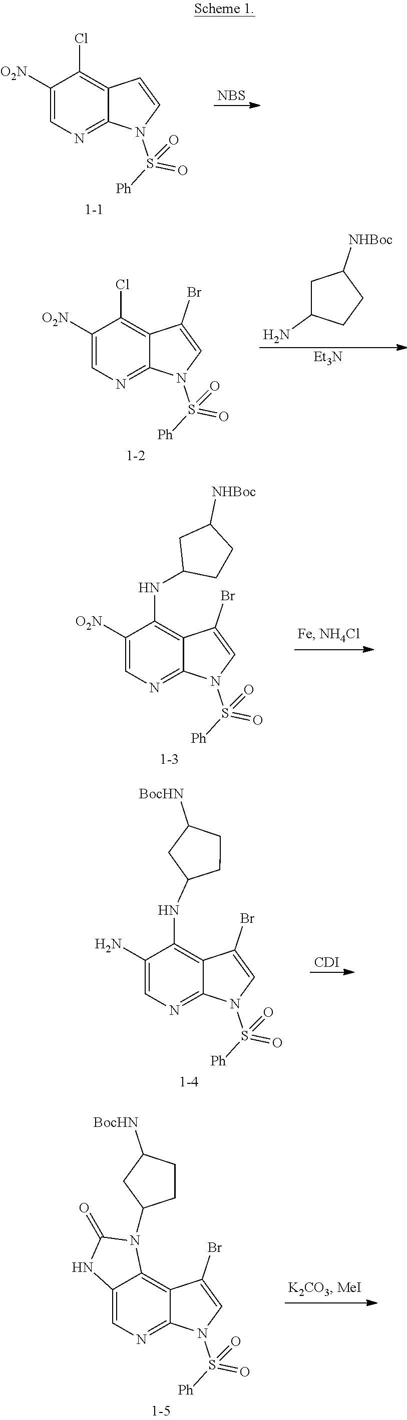

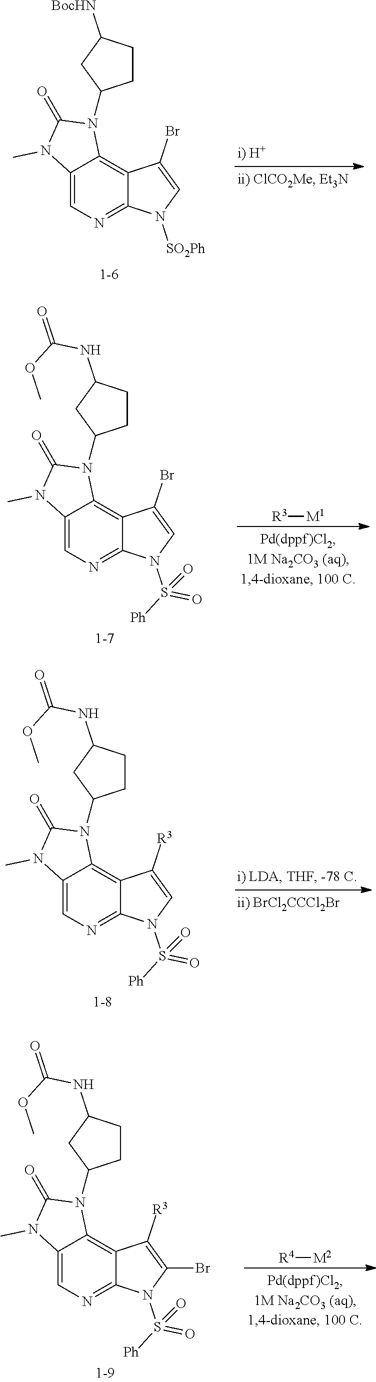

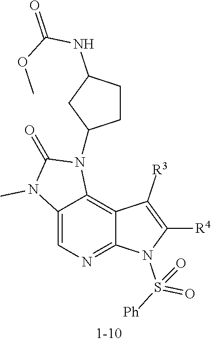

In the case where R⁴ of general structure 1-10 has an ester moiety attached, as exemplified by 2-1 and 2-4, the ester and N-phenyl sulphonyl groups can be hydrolyzed using an appropriate base, such as a metal hydroxide (e.g. LiOH, NaOH, etc.) to afford the carboxylic acids of general structure 2-2 and 2-5, respectively as outlined in Scheme 2. Alternatively, as can be appreciated by one skilled in the art, the ester moiety could by hydrolyzed chemoselectively in the presence of the phenyl sulfonyl protecting group utilizing a variety of conditions and reagents, such as TMSI. Furthermore, the phenyl sulfonyl protecting group on the indole-like nitrogen could be exchanged for a non-base sensitive protecting group, such as SEM. Amide coupling can be achieved using a coupling reagent (e.g. HATU, BOP, DCC, etc.) in the presence of a base, such as Hunig's base, and the appropriate amine and solvent (e.g. DCE, DMF, etc.). Alternatively, the carboxylic acid can be converted to the corresponding acyl chloride, using a reagent such as oxalyl chloride, thionyl chloride, cyanuric chloride, etc. followed by reaction with the appropriate amine in the presence of base. The amine used in the amide coupling may contain no linker, partial or full linker, or linker attached to an E3 ligase recruiter. The substituents on the amine of the amide coupling reaction, R^{a} and R^{b}, can be independently selected or joined to form a heterocycle, such as an azetidine, piperdine, piperazine, etc. In cases where R^{a} and R^{b} contain amines, alcohols, or carboxylic acids, they may be protected with the appropriate protecting groups prior to the amide coupling reaction.

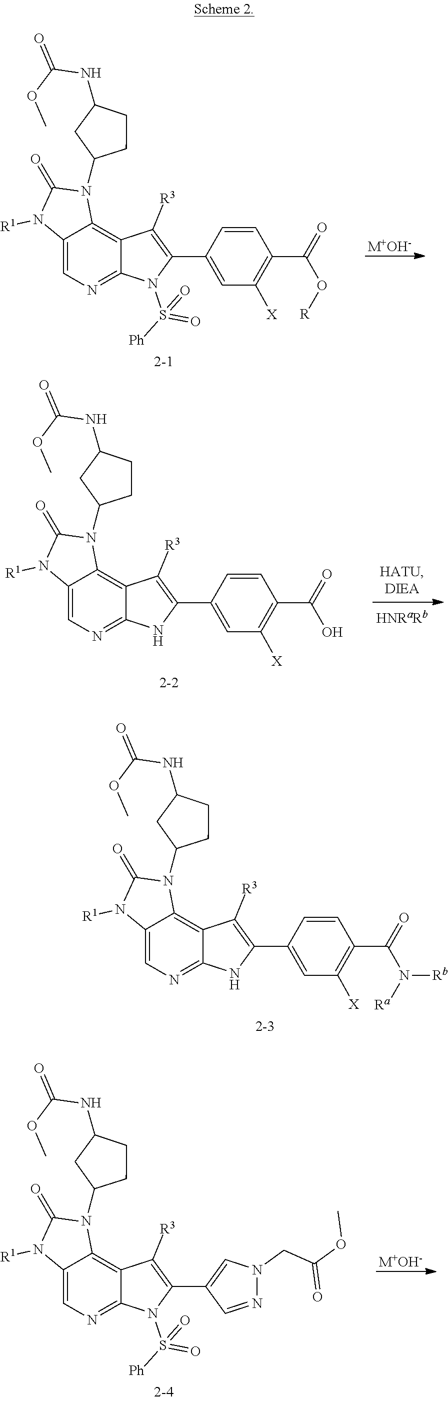

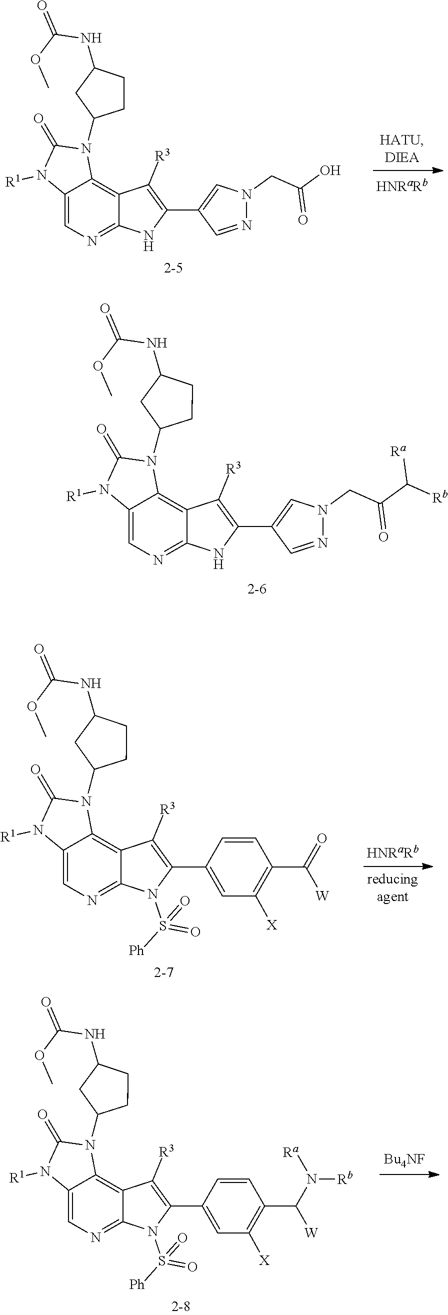

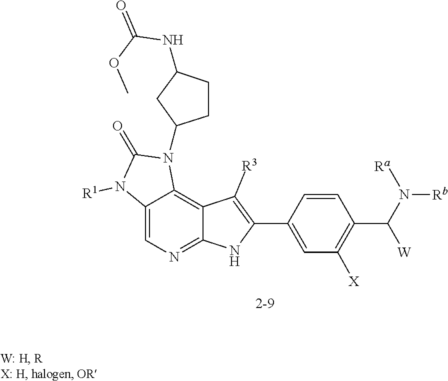

In the case where R⁴ of general structure 1-10 contains an aldehyde or ketone, such as 2-7, reductive amination can be conducted by reacting 2-7 with an appropriate amine in the presence of a reducing agent (e.g. sodium triacetoxyborohydride, sodium cyanoborohydride, trimethyl borate). The amine used in the reductive amination may contain no linker, partial or full linker, or linker attached to an E3 ligase recruiter. The substituents on the amine R^{a} and R^{b} can be independently selected or joined to form a heterocycle, such as an azetidine, piperdine, piperazine, etc. In cases where R^{a} and R^{b} contain amines, alcohols, or carboxylic acids, they may be protected with the appropriate protecting groups prior to the reductive amination reaction. The phenyl sulfonyl protecting group can be removed using standard conditions, such as treatment with tetra-n-butylammonium fluoride (TBAF), magnesium in the presence of ammonium chloride in methanol, or sodium tert-butoxide. Any protecting groups present on R^{a} and/or R^{b} may also be removed using methods known to one skilled in the art.

In the case where R⁴ of general structure 1-10 contains an aromatic or heteroaromatic ring that is substituted with a halogen (e.g., Cl, Br, or I) or pseudohalogen (e.g., OTf or OMs), such as 3-1, wherein A and W can be a carbon or nitrogen, one skilled in the art of organic synthesis, can conduct a variety of coupling reactions to afford products outlined in Scheme 3. In cases where A and/or W is nitrogen, S_{N}Ar reactions can be conducted with the appropriate amine or alcohol in the presence of a base to afford compounds 3-4 and 3-6, respectively. Amine 3-4 can also be prepared by a variety of known amine coupling reactions, such as Buchwald, Chan-Lam, and nickel-catalyzed amination reactions (Garg, N. K, et al. ACS Catal. 2014, 4, 3289). In cases where A: nitrogen and X: OH for 3-1, amide coupling reagents, such as BOP and HATU, may be implemented to form the C—N bond of 3-4 (Mansour, T. et al. Org. Lett. 2006, 8, 2425). Ether 3-6 can also be prepared from the corresponding alcohol 3-5 using Williamson ether synthesis, or other methods known to one skilled in the art of organic synthesis. In another embodiment, 3-1 can participate in deoxygenative (Liu, W., Mulhearn, J., et al. ACS Med. Chem. Lett. 2023, 14, 853) or decarboxylative (Baran, P. S., et al. ACS Med. Chem. Lett. 2022, 13, 1413.) C(sp²)—C(sp³) cross coupling reactions, wherein R^{a} and R^{b} can join to form a cycloalkyl or heterocycle. Compound 3-1 can be coupled with a boronic acid, boronate ester, potassium trifluoroborate, or an appropriately substituted metal such as Sn(Bu)₃ or Zn, under standard Suzuki-Miyaura conditions (e.g., in the presence of a palladium catalyst, such as tetrakis(triphenylphosphine)palladium(O) and a base (e.g., a carbonate base)) or standard Stille conditions (e.g., in the presence of a palladium(O) catalyst, such as tetrakis(triphenylphosphine)palladium(O)) or standard Negishi conditions (e.g., in the presence of a palladium catalyst, such as tetrakis(triphenylphosphine)palladium(O) or [1,1′-bis(diphenylphosphino)ferrocene]dichloropalladium (II)), to afford compound 3-3.

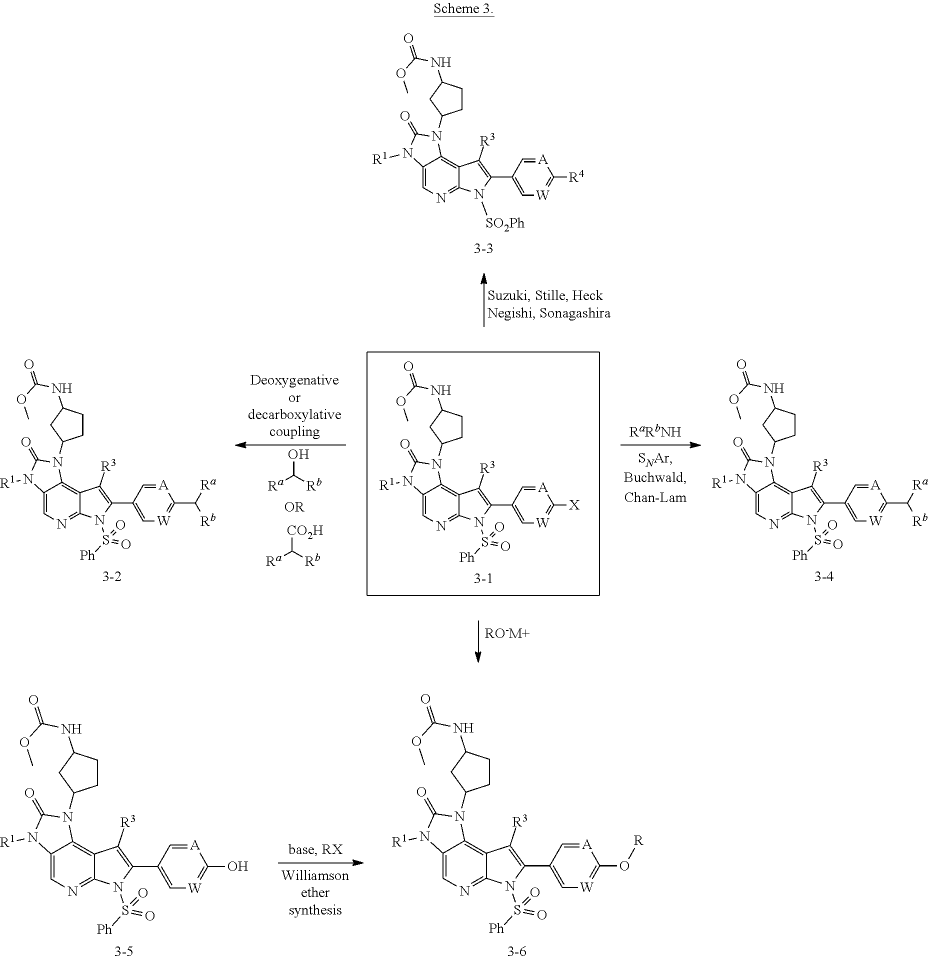

The synthesis of the linker portion of the molecule that is attached to the E3 ligase recruiter can be prepared by a variety of methods, such as those described in: Sosic, I; et al. Front. Chem. 2021, 9, 707317; Han, X. and Sun, Y; Med. Comm. 2023, 4, e290; Hayhow, T. G; et al. Chem. Eur. J. 2020, 26, 16818. As one skilled in the art can appreciate, there may be instances in which it is advantageous to attach the linker to the JAK2 inhibitor followed by attachment to the E3 ligase and some instances where the reverse order is preferred or performed. One example of coupling the linker to the E3 ligase is outlined in Scheme 4 for the synthesis of 4-6, which comprises of an Inhibitor of Apoptosis (IAP) E3 ligase recruiter. This scheme illustrates the synthetic strategy of utilizing differentiated protecting groups on the linker and E3 ligase containing molecules, as illustrated by 4-1 and 4-2. It should be noted that R^{c} and L can be linked together to form a cycle, bicycle, spiro cycle, etc. (e.g. piperdine, piperazine, azetidine, etc.) One could utilize amines, such as 4-4, in variety of chemistry, such as reductive amination, S_{N}Ar, Buchwald coupling, and amide coupling, as illustrated in Scheme 4, to make compounds such as 4-5. Any remaining protecting groups can be removed using methods known to one skilled in the art and as described by T. W. Greene and P. G. M. Wuts, Protective Groups in Organic Synthesis, 3^{rd} Ed., Wiley & Sons, Inc., New York (1999).

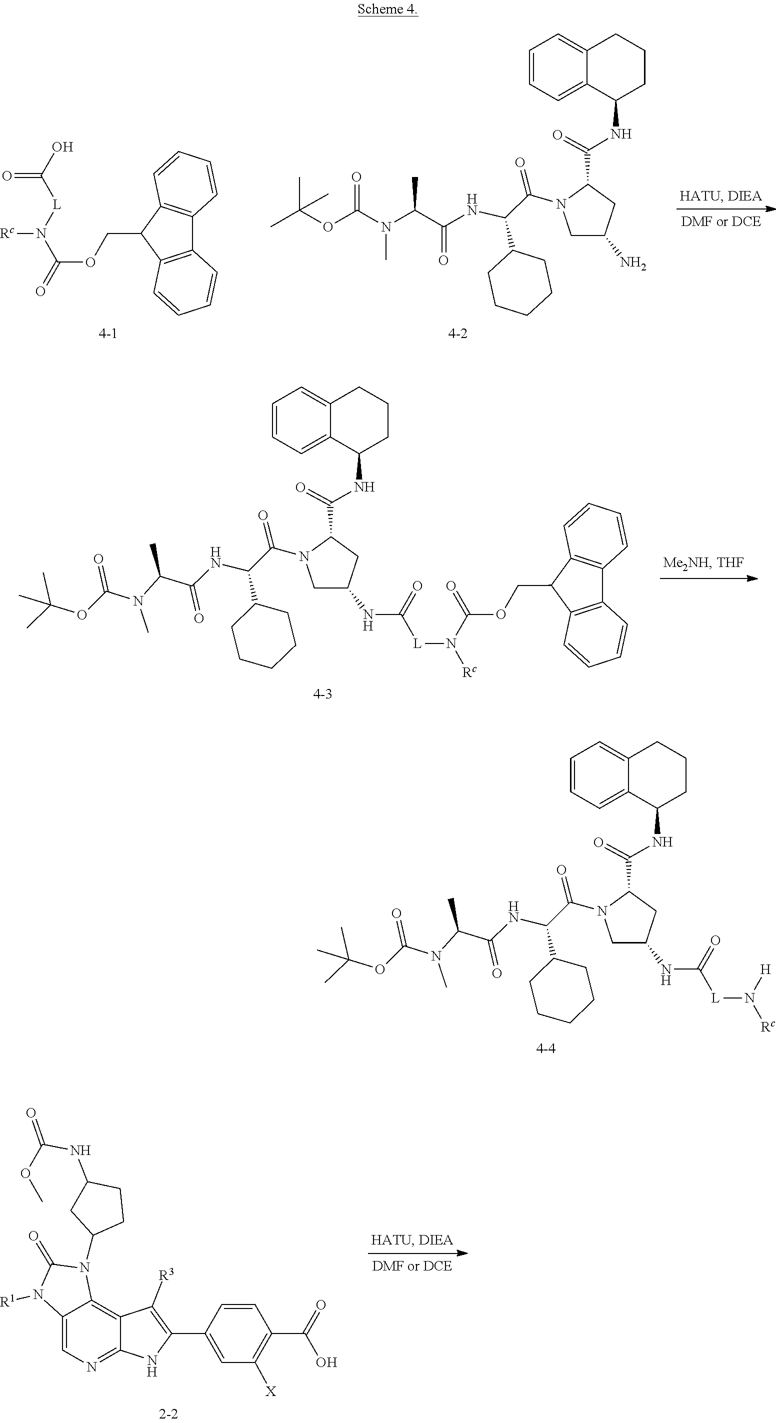

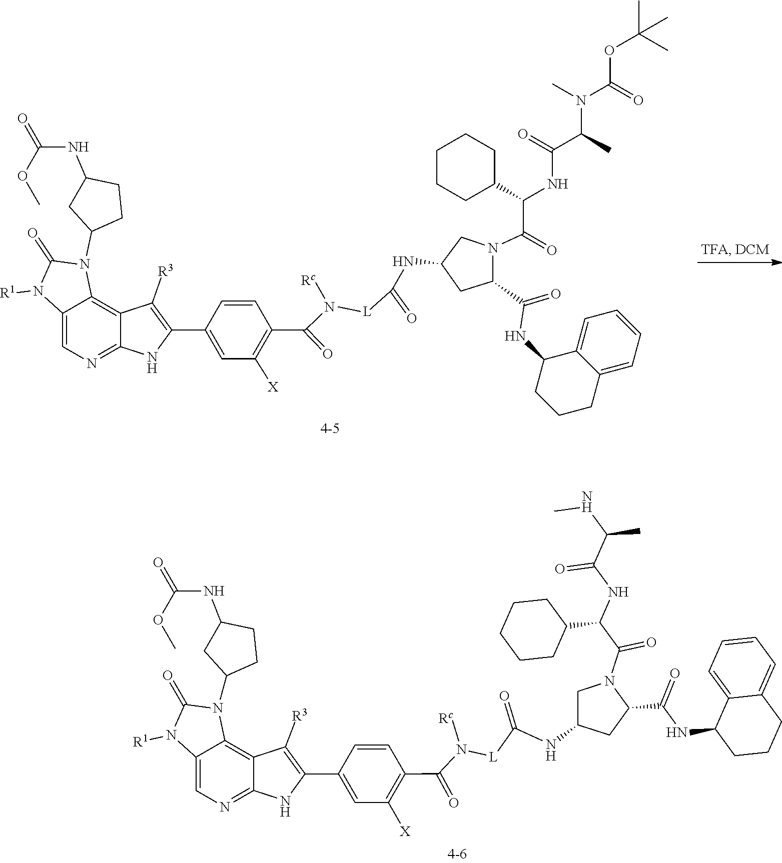

Cereblon recruiters can be prepared in a variety of manners known to one skilled in the art, as well as methods described in the references disclosed above and depicted in Scheme 5. Cereblon starting materials containing an amine, such as 5-2, can be reacted with an aldehyde or ketone using reductive amination protocols or with a carboxylic acid III using standard amide coupling procedures. It should be noted that R^{d} and L of 5-1 can be linked together to form a cycle, bicycle, spiro cycle, etc. In the case where the cereblon recruiter contains a halogen, such as 5-5, one can perform S_{N}Ar reactions when X: F or Buchwald coupling methods in the case where X: Br, such as those described by Hayhow, T. G; et al. Chem. Eur. J. 2020, 26, 16818. In cases where X: Cl or —OSO₂NMe₂, nickel-catalyzed amination can be performed as described by Garg, N. K, et al. ACS Catal. 2014, 4, 3289. It should be noted that the R^{d} and/or R^{e} of 5-6, 5-7, and 5-8 can be linked with L to form a cycle, bicycle, spiro cycle, etc. Once the cereblon portion of the molecule is appended to the linker (e.g. 5-3 and 5-7), the terminal end of the linker can be deprotected and subsequently tethered to the JAK inhibitor as outlined in Scheme 2.

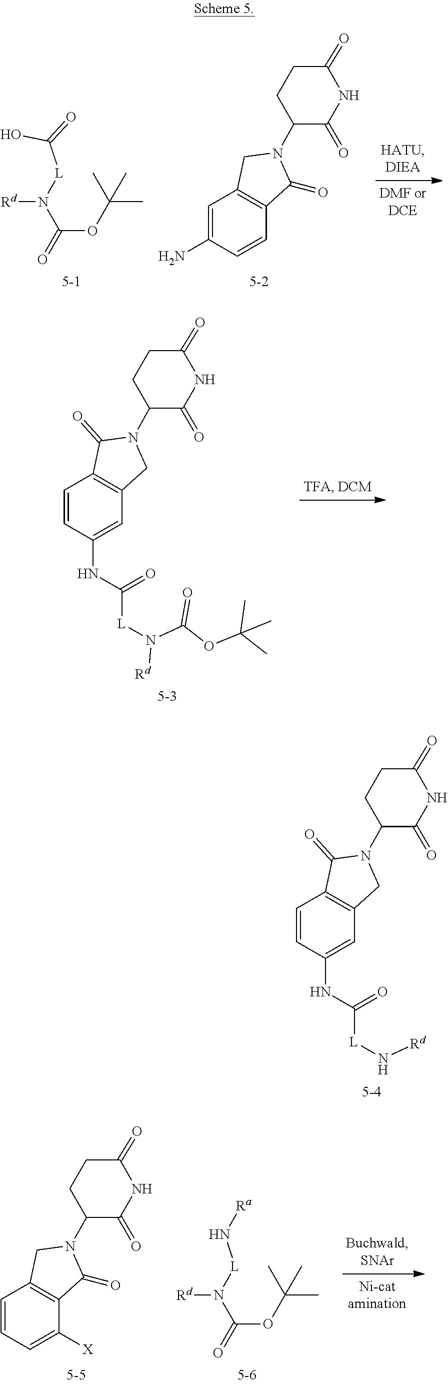

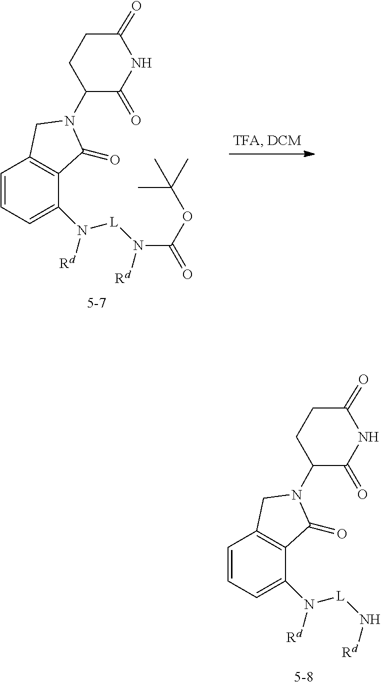

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can bind to the pseudokinase domain of JAK2 and inhibit the activity of or degrade JAK2 to regulate aberrant JAK-STAT signaling. In certain embodiments, inhibition or degradation of mutated JAK2 (such as V617F JAK2) and/or JAK2 WT may be useful in providing a means of regulating overactivated JAK-STAT signaling that is implicated in the pathogenesis of a variety of cancers, inflammatory, and autoimmune diseases.

In certain embodiments, the present disclosure provides a method for treating a V617F-related disorder in a patient in need thereof, comprising the step of administering to said patient a compound of the disclosure, or a pharmaceutically acceptable composition thereof.

Myeloproliferative diseases (MPD) are multipotent hematopoietic stem cell disorders characterized by excess production of various blood cells. MPNs include polycythemia vera (PV), essential thrombocythemia (ET), and idiopathic myelofibrosis (IMF). JAK2 V617F mutation is reported in about 95% of patients with PV, in 35% to 70% of patients with ET, and 50% of patients with IMF. Also, JAK2 exon 12 mutations are detected in some of the V617F-negative PV patients (Ma et al., J. Mol. Diagn., 11: 49-53, 2009). In some embodiments, the compounds of the disclosure can be useful in the treatment of myeloproliferative disorders (e.g., myeloproliferative neoplasms) in a patient in need thereof, such as polycythemia vera, essential thrombocythemia, myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like.

In some embodiments, the myeloproliferative disorder is selected from polycythemia vera, essential thrombocythemia, myelofibrosis with myeloid metaplasia, primary myelofibrosis, post-essential thrombocythemia myelofibrosis, and post polycythemia vera myelofibrosis.

In some embodiments, the myeloproliferative disorder is a myeloproliferative neoplasm.

In some embodiments, the myeloproliferative disorder is myelofibrosis (e.g., primary myelofibrosis (PMF) or post polycythemia vera/essential thrombocythemia myelofibrosis (Post-PV/ET MF)).

In some embodiments, the myeloproliferative disorder is primary myelofibrosis (PMF).

In some embodiments, the myeloproliferative disorder is post-essential thrombocythemia myelofibrosis (Post-ET MF).

In some embodiments, the myeloproliferative disorder is post polycythemia vera myelofibrosis (Post-PV MF).

In some embodiments, the myeloproliferative disorder is selected from primary myelofibrosis (PMF), polycythemia vera (PV), and essential thrombocythemia (ET).

In some embodiments, the myeloproliferative neoplasm is primary myelofibrosis (PMF).

In some embodiments, the myeloproliferative neoplasm is polycythemia vera (PV).

In some embodiments, the myeloproliferative neoplasm is essential thrombocythemia (ET).

Myeloproliferative diseases include disorders of a bone marrow or lymph node-derived cell type, such as a white blood cell. A myeloproliferative disease can manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CMIL), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

For example, the compounds of the disclosure are useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphomas, and Burkitt's lymphoma.

In certain embodiments, provided herein is a method of treating cancer comprising administering to a patient in need thereof a therapeutically effect amount of a compound of the disclosure. In certain embodiments, the cancer is selected from T lymphoblastic lymphoma, glioblastoma, melanoma, rhabdosarcoma, lymphosarcoma, and osteosarcoma.

Other cancers treatable with the compounds of the disclosure include tumors of the eye, glioblastoma, melanoma, leiomyosarcoma, and urothelial carcinoma (e.g., ureter, urethra, bladder, urachus).

The compounds of the disclosure can also be useful in the inhibition or degradation of tumor metastases.

In some embodiments, the compounds of the disclosure as described herein can be used to treat Alzheimer's disease, HIV, or tuberculosis.

In some embodiments, the compounds of the disclosure can be useful in the treatment of myelodysplastic syndrome (MDS) in a patient in need thereof. In some embodiments, said patient having the myelodysplastic syndrome (MDS) is red blood cell transfusion dependent.

As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥ 10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × | Dysplasia in ≥ 10% of cells |
| multilineage dysplasia | 10⁹/L monocytes | in ≥ 2 hematopoietic lineages, |
| | | ±15% ring sideroblasts, < 5% |
| | | blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ±Auer rods, 10% to |
| | monocytes | 19% blasts |
| Myelodysplastic | Cytopenias | Unilineage or no dysplasia but |
| syndrome, unclassified | | characteristic MDS |
| (MDS-U) | | cytogenetics, <5% blasts |
| MDS associated with | Anemia, platelets | Unilineage erythroid. Isolated |
| isolated del(5q) | normal or increased | del(5q), <5% blasts |

In some embodiments, the myelodysplastic syndrome is refractory cytopenia with unilineage dysplasia (RCUID).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, the compounds of the disclosure can be useful in the treatment of leukemia.

In some embodiments, the compounds of the disclosure can be useful in the treatment of acute myeloid leukemia (AML).

In addition to oncogenic neoplasms, the compounds of the disclosure can be useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes.

The compounds provided herein may further be useful in the treatment of inflammatory and immune disorders, including, but not limited to, rheumatoid arthritis, psiruatic arthritis, ulcerative colitis, ankylosing spondylosis, juvenile idiopathic arthritis, atopic dermatitis, vitiligo, alopecia areata, and graft versus host disease (GvHD).

The compounds provided herein may further be useful in the treatment of fibrotic diseases, such as where a disease symptom or disorder is characterized by fibrosis.

Example fibrotic diseases include liver cirrhosis, glomerulonephritis, pulmonary fibrosis, systemic fibrosis, rheumatoid arthritis, and wound healing.

In some embodiments, the inflammatory and immune disorder is selected from rheumatoid arthritis and atopic dermatitis (see e.g., Leroy, E. et al. J. Allergy Clin. Immunol. 2019, 144, 224; Adel, Y. et al. EJIM, 2021, 33:56; and Kawakami, T. et al. Front. Immun. 2015, 6, 434).

In some embodiments, the inflammatory and immune disorder is rheumatoid arthritis.

In some embodiments, the inflammatory and immune disorder is atopic dermatitis.

In some embodiments, the compounds provided herein can be used in the treatment of a hypophosphatemia disorder such as, for example, X-linked hypophosphatemic rickets, autosomal recessive hypophosphatemic rickets, and autosomal dominant hypophosphatemic rickets, or tumor-induced osteomalacia.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a compound described herein includes the administration of a compound described herein to an individual or patient, such as a human, having a V617F variant, as well as, for example, introducing a compound described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed; Lippincott Williams & Wilkins: Philadelphia, Pa., 2005; Handbook of Pharmaceutical Excipients, 6th ed; Rowe et al., Eds; The Pharmaceutical Press and the American Pharmaceutical Association: 2009; Handbook of Pharmaceutical Additives, 3rd ed; Ash and Ash Eds; Gower Publishing Company: 2007; Pharmaceutical Preformulation and Formulation, 2nd ed; Gibson Ed; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment or prevention of V617F-associated diseases, disorders or conditions, or diseases or conditions as described herein. The agents can be combined with the present compounds in a single dosage form, or the agents can be administered simultaneously or sequentially as separate dosage forms.

Compounds described herein can be used in combination with one or more other kinase inhibitors or degraders for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor or degraders as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD19, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein can be used in combination with targeted therapies, including JAK kinase inhibitors (ruxolitinib, additional JAK1/2 and JAK1-selective, baricitinib, povorcitinib, or itacitinib), Pim kinase inhibitors (e.g., LGH447, INCB053914 and SGI-1776), PI3 kinase inhibitors including PI3K-delta selective and broad spectrum PI3K inhibitors (e.g., parsaclisib and INCB50797), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Ax1, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibitors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC₅₀ less than 1 μM, less than 100 nM, less than 10 nM or less than 1 nM in a PD-L1 assay described in US Patent Publication Nos. US 20170107216, US 20170145025, US 20170174671, US 20170174679, US 20170320875, US 20170342060, US 20170362253, and US 20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is retifanlimab (also known as MGA012), nivolumab, pembrolizumab (also known as MK-3475), pidilizumab, SHR-1210, PDR001, ipilumumab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is retifanlimab. In some embodiments, the anti-PD1 antibody is SHR-1210. Other anti-cancer agent(s) include antibody therapeutics such as 4-1BB (e.g. urelumab, utomilumab.

In some embodiments, the compounds of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure can be used in combination with one or more metabolic enzyme inhibitors. In some embodiments, the metabolic enzyme inhibitor is an inhibitor of IDO₁, TDO, or arginase. Examples of IDO₁ inhibitors include epacadostat, NLG919, BMS-986205, PF-06840003, IOM2983, RG-70099 and LY338196.

In some embodiments, the compounds described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC₁₋₆₇₅₄₅₁) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-I 549. Other antiviral agents include hydroxyurea, ribavirin, IL-2, IL-12, pentafuside and Yissum Project No. 11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, degraders, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors or degraders by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula I can be optionally substituted with deuterium atoms, such as —CD₃ (i.e., trideuteromethyl) being substituted for —CH₃). In some embodiments, alkyl groups of the disclosed Formulas (e.g., Formula I, Formula Ia, etc.) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6,1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkoxy, phenyl, and indazolyl substituents, as described herein, is optionally replaced by deuterium atoms.

In some embodiments, each hydrogen atom of the compounds provided herein, such as hydrogen atoms to carbon atoms of alkyl, alkoxy, phenyl, and indazolyl substituents, as described herein, is replaced by deuterium atoms (i.e., the alkyl, alkoxy, phenyl, and indazolyl substituents are perdeuterated).

In some embodiments, 1, 2, 3, 4, 5, 6, 7, 8, 9, 10, 11, or 12 hydrogen atoms, attached to carbon atoms of alkyl, alkoxy, phenyl, and indazolyl substituents, as described herein, are optionally replaced by deuterium atoms.

In some embodiments, 1, 2, 3, 4, 5, 6, 7, or 8 hydrogen atoms, attached to carbon atoms of alkyl, alkoxy, phenyl, and indazolyl substituents, as described herein, are optionally replaced by deuterium atoms.

In some embodiments, the compound provided herein (e.g., the compound of any of Formulas I-Vd), or a pharmaceutically acceptable salt thereof, comprises at least one deuterium atom.

In some embodiments, the compound provided herein (e.g., the compound of any of Formulas I-Vd), or a pharmaceutically acceptable salt thereof, comprises two or more deuterium atoms.

In some embodiments, the compound provided herein (e.g., the compound of any of Formulas I-Vd), or a pharmaceutically acceptable salt thereof, comprises three or more deuterium atoms.

In some embodiments, the compound provided herein (e.g., the compound of any of Formulas I-Vd), or a pharmaceutically acceptable salt thereof, comprises three to nine deuterium atoms.

In some embodiments, for a compound provided herein (e.g., the compound of any of Formulas I-Vd), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of V617F-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

E3 ligase recruiters (E) were purchased from a variety of vendors' compound libraries, such as Biotechne/Tocris' PROTAC® Degraders & Targeted Protein Degradation library, Enamine's Protein Degrader Toolbox library, Millipore Sigma's (Aldrich) Targeted Protein Degradation libraries, or prepared using methods known by one skilled in the art, such as those outlined in Bricelj, A. et al. Front. Chem. 2021, 9, 707317 and International Application Publication No.: WO 2020/2000291A1, the disclosure of each of which is incorporated herein by reference in its entirety.

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)).

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:
pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

pH=10 purifications: Waters XBridge™ C₁₈ 5 μm, 30×100 mm column, eluting with mobile phase A: 0.1% NH₄₀H in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

### Example 1. Methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

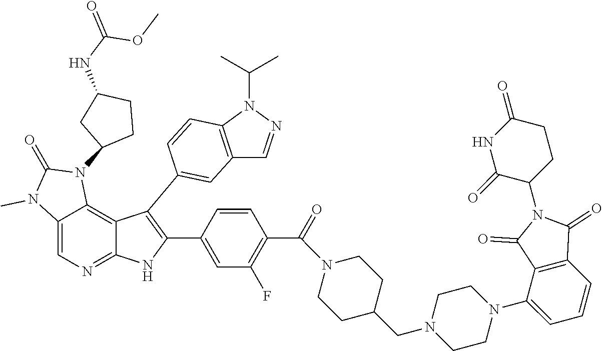 [SMILES: COC(=O)N[C@@H]1CC[C@@H](n2c(=O)n(C)c3cnc4[nH]c(-c5ccc(C(=O)N6CCC(CN7CCN(c8cccc9c8C(=O)N(C8CCC(=O)NC8=O)C9=O)CC7)CC6)c(F)c5)c(-c5ccc6c(cnn6C(C)C)c5)c4c32)C1]

### Step 1. 3-Bromo-4-chloro-5-nitro-1-(phenylsulfonyl)-1H-pyrrolo[2,3-b]pyridine

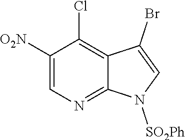

To a solution of 4-chloro-5-nitro-1-(phenylsulfonyl)-1H-pyrrolo[2,3-b]pyridine (5.00 g, 14.8 mmol; eNovation Chemicals, D572641) in dry dimethylformamide (30 mL), N-bromosuccinimide (5.00 g, 28.1 mmol) was added at room temperature. The resulting mixture was stirred for 60 minutes at 60° C. The yellow reaction mixture was cooled to 0° C. and kept at the same temperature for 6 hours. The resulting precipitate was collected using filtration followed by the wash with hexanes to give the desired product as a light yellow solid (5.35 g, 87.2%). LCMS for C₁₃H₈BrClN₃O₄S (M+H)⁺: m/z=415.9, 417.9; Found: 415.9, 417.9. ¹H NMR (500 MHz, DMSO) δ 9.06 (s, 1H), 8.54 (s, 1H), 8.19 (d, J=7.8 Hz, 2H), 7.80 (t, J=7.8 Hz, 1H), 7.68 (t, J=7.8 Hz, 2H).

### Step 2. tert-Butyl((1R,3R)-3-((3-bromo-5-nitro-1-(phenylsulfonyl)-1H-pyrrolo[2,3-b]pyridin-4-yl)amino)cyclopentyl)carbamate

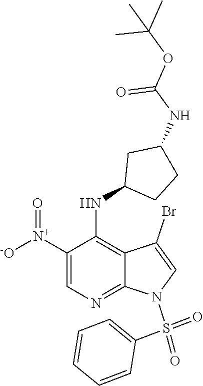

A mixture of 3-bromo-4-chloro-5-nitro-1-(phenylsulfonyl)-1H-pyrrolo[2,3-b]pyridine (5.50 g, 13.2 mmol), triethylamine (3.68 mL, 26.4 mmol), and tert-butyl((1R,3R)-3-aminocyclopentyl)carbamate (2.88 g, 14.4 mmol) in acetonitrile (66 mL) was stirred at 70° C. The heterogeneous solution quickly turned homogeneous, followed by the formation of a heavy precipitate. After stirring for 30 min., the precipitate was filtered off and washed with ACN/H₂O (1:2). The solids were collected and placed under high vacuum to afford 7.51 g (98% yield). LCMS for C₂₃H₂₇BrN₅O₆S (M+H)⁺: m/z=580.1, 582.1; Found: 580.1, 582.1.

### Step 3. tert-Butyl((1R,3R)-3-((5-amino-3-bromo-1-(phenylsulfonyl)-1H-pyrrolo[2,3-b]pyridin-4-yl)amino)cyclopentyl)carbamate

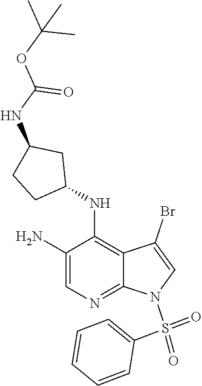 [SMILES: CC(C)(C)OC(=O)N[C@@H]1CC[C@@H](Nc2c(N)cnc3c2c(Br)cn3S(=O)(=O)c2ccccc2)C1]

A mixture of tert-butyl((1R,3R)-3-((3-bromo-5-nitro-1-(phenylsulfonyl)-1H-pyrrolo[2,3-b]pyridin-4-yl)amino)cyclopentyl)carbamate (7.51 g, 12.9 mmol), ammonium chloride (10.4 g, 194 mmol), and iron (10.8 g, 194 mmol) in THF (80 mL) and water (80 mL) was heated at 80° C. After stirring overnight, the reaction mixture was diluted with ethyl acetate (150 mL) and filtered over a pad of Celite™. The inorganics were thoroughly washed with ethyl acetate. The organic phase was washed with brine, dried over sodium sulfate, and concentrated in-vacuo to afford 7.12 g of the desired product (100% yield), which was used in the subsequent step without further purification. LCMS for C₂₃H₂₉BrN₅O₄S (M+H)⁺: m/z=550.1, 552.1; Found: 550.2, 552.2.

### Step 4. tert-Butyl((1R,3R)-3-(8-bromo-2-oxo-6-(phenylsulfonyl)-3,6-dihydroimidazo[4,5-d]pyrrolo[2,3-b]pyridin-1(2H)-yl)cyclopentyl)carbamate

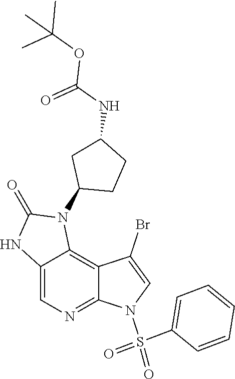 [SMILES: CC(C)(C)OC(=O)N[C@@H]1CC[C@@H](n2c(=O)[nH]c3cnc4c(c(Br)cn4S(=O)(=O)c4ccccc4)c32)C1]

To a mixture of tert-butyl((1R,3R)-3-((5-amino-3-bromo-1-(phenylsulfonyl)-1H-pyrrolo[2,3-b]pyridin-4-yl)amino)cyclopentyl)carbamate (3.58 g, 6.50 mmol) in acetonitrile (70 mL) was added 1,1′-carbonyldiimidazole (2.11 g, 13.0 mmol) and the resulting mixture was heated at 65° C. After 3 h, the reaction was deemed complete and allowed to stir overnight. The precipitate was filtered off, washed with acetonitrile, and dried under high vacuum to afford 2.62 g of desired product. The filtrate was purified by CombiFlash chromatography (120 g silica gel column, eluting with 0-10% MeOH/DCM) to afford an additional 0.345 g of product (79% yield). LCMS for C₂₄H₂₇BrN₅O₅S (M+H)⁺: m/z=576.1, 578.1; Found: 576.1, 578.1.

### Step 5. tert-Butyl((1R,3R)-3-(8-bromo-3-methyl-2-oxo-6-(phenylsulfonyl)-3,6-dihydroimidazo[4,5-d]pyrrolo[2,3-b]pyridin-1(2H)-yl)cyclopentyl)carbamate

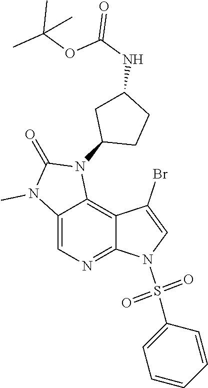 [SMILES: Cn1c(=O)n([C@@H]2CC[C@@H](NC(=O)OC(C)(C)C)C2)c2c3c(Br)cn(S(=O)(=O)c4ccccc4)c3ncc21]

To a mixture of tert-butyl((1R,3R)-3-(8-bromo-2-oxo-6-(phenylsulfonyl)-3,6-dihydroimidazo[4,5-d]pyrrolo[2,3-b]pyridin-1(2H)-yl)cyclopentyl)carbamate (2.74 g, 4.75 mmol) and cesium carbonate (3.10 g, 9.51 mmol) in THF (25 mL) and acetonitrile (25 mL) was added iodomethane (1.2 mL, 19 mmol) and the mixture was stirred at 60° C. for several hours. Upon cooling to ambient temperature, the crude reaction mixture was diluted with EtOAc, filtered through a pad of Celite™, washed with EtOAc, and concentrated in-vacuo. The residue was purified by CombiFlash chromatography (120 g silica gel column, eluting with 65-100% EtOAc/hexanes) to afford the desired product (2.39 g, 85% yield). LCMS for C₂₅H₂₉BrN₅O₅S (M+H)⁺: m/z=590.1, 592.1; Found: 590.1, 592.1.

### Step 6. Methyl((1R,3R)-3-(8-bromo-3-methyl-2-oxo-6-(phenylsulfonyl)-3,6-dihydroimidazo[4,5-d]pyrrolo[2,3-b]pyridin-1(2H)-yl)cyclopentyl)carbamate

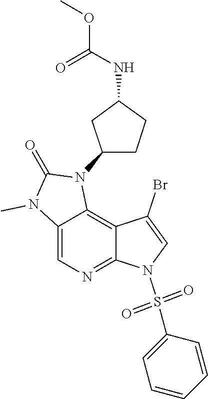 [SMILES: COC(=O)N[C@@H]1CC[C@@H](n2c(=O)n(C)c3cnc4c(c(Br)cn4S(=O)(=O)c4ccccc4)c32)C1]

To a solution of tert-butyl((1R,3R)-3-(8-bromo-3-methyl-2-oxo-6-(phenylsulfonyl)-3,6-dihydroimidazo[4,5-d]pyrrolo[2,3-b]pyridin-1(2H)-yl)cyclopentyl)carbamate (2.355 g, 3.99 mmol) in DCM (50 mL) was added TFA (12 mL, 156 mmol) and the solution was stirred at ambient temperature for 5 h. The volatiles were removed in-vacuo and the residue was azeotropically washed with acetonitrile several times. The crude residue was dissolved in DCM (50 ml) and cooled to 0° C. prior to the sequential addition of triethylamine (2.78 mL, 19.9 mmol) and methyl chloroformate (0.772 mL, 9.97 mmol). The solution was allowed to gradually warm to ambient temperature and stirred for 3 h. The reaction mixture was concentrated in-vacuo and purified by CombiFlash chromatography (120 g silica gel column, eluting with 65-100% EtOAc/hexanes) to afford the desired product (1.82 g, 85% yield). LCMS for C₂₂H₂₃BrN₅O₅S (M+H)⁺: m/z=548.1, 550.1; Found: 548.1, 550.1.

### Step 7. Methyl((1R,3R)-3-(8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate

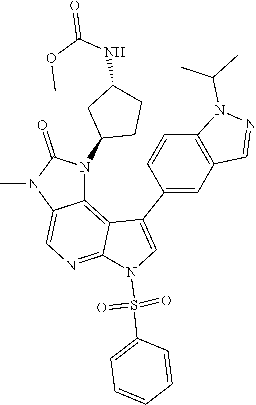 [SMILES: COC(=O)N[C@@H]1CC[C@@H](n2c(=O)n(C)c3cnc4c(c(-c5ccc6c(cnn6C(C)C)c5)cn4S(=O)(=O)c4ccccc4)c32)C1]

A 40-mL vial containing methyl((1R,3R)-3-(8-bromo-3-methyl-2-oxo-6-(phenylsulfonyl)-3,6-dihydroimidazo[4,5-d]pyrrolo[2,3-b]pyridin-1(2H)-yl)cyclopentyl)carbamate (205 mg, 0.374 mmol), (1-isopropyl-1H-indazol-5-yl)boronic acid (196 mg, 0.961 mmol, Combi-Blocks BB-0404 [1551416-99-9]), Na₂CO₃ (1.0 M in water) (1.12 mL, 1.12 mmol) and PdCl₂(dppf)-CH₂Cl₂-adduct (30.5 mg, 0.037 mmol) was degassed and purged several times with N₂ prior to heating at 100° C. After 2 h, the reaction mixture was diluted with ethyl acetate and filtered over a pad of Celite™. The layers were separated and the aqueous layer was extracted with ethyl acetate several times. The combined organic layers were washed with brine, dried over Na₂SO₄ and concentrated in-vacuo to afford the crude product as a dark brown oil. The crude residue was purified by CombiFlash chromatography (40 g silica gel column, eluting with 65-100% EtOAc/hexanes) to afford the desired product (0.193 g, 82% yield). LCMS for C₃₂H₃₄N₇O₅S (M+H)⁺: m/z=628.2; Found: 628.3.

### Step 8. Methyl((1R,3R)-3-(7-bromo-8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate

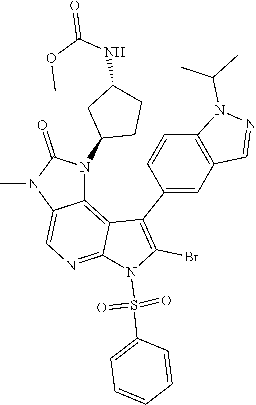 [SMILES: COC(=O)N[C@@H]1CC[C@@H](n2c(=O)n(C)c3cnc4c(c(-c5ccc6c(cnn6C(C)C)c5)c(Br)n4S(=O)(=O)c4ccccc4)c32)C1]

A solution of methyl((1R,3R)-3-(8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate (292 mg, 0.465 mmol) in anhydrous THF (4 mL) was cooled to −78° C. prior to the drop-wise addition of LDA 2.0 M THF/heptane/ethylbenzene (0.65 mL, 1.3 mmol, 2.8 equiv.) under an atmosphere of N₂ (g). After stirring for 40 min. at −78° C., an anhydrous solution of 1,2-dibromo-1,1,2,2-tetrachloroethane (151 mg, 0.465 mmol) in THF (0.5 mL) was added drop-wise to the reaction. After stirring for 2 h at −78° C., the reaction was quenched by the addition of saturated NH₄Cl (0.034 mL, aqueous), warmed to ambient temperature, concentrated in-vacuo, and injected onto a 12 g silica gel column and purified by CombiFlash chromatography (40 g silica gel column, eluting with 70-100% EtOAc/hexanes) to afford the desired product 275 mg (84% yield). LCMS for C₃₂H₃₃BrN₇O₅S (M+H)⁺: m/z=706.1, 708.1; Found: 706.2, 708.2.

### Step 9. Methyl 2-fluoro-4-(8-(1-isopropyl-1H-indazol-5-yl)-1-((R,3R)-3-((methoxycarbonyl)amino)cyclopentyl)-3-methyl-2-oxo-6-(phenysulfonyl)-1,2,3,6-tetrahydroimidazo[4,5-d]pyrrolo[2,3-b]pyridin-7yl)benzoate

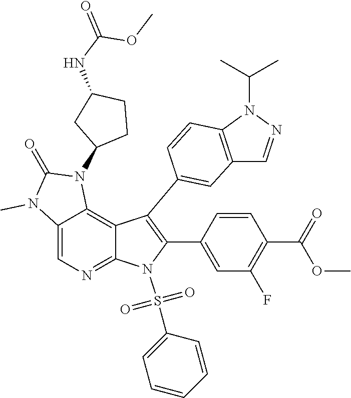

A mixture of (3-fluoro-4-(methoxycarbonyl)phenyl)boronic acid (58.8 mg, 0.297 mmol, Combi-Blocks BB-2719, [505083-04-5]), methyl((1R,3R)-3-(7-bromo-8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate (70 mg, 0.099 mmol), PdCl₂(dppf)-CH₂Cl₂ adduct (16.2 mg, 0.0200 mmol), and 1M K₂CO₃ (aq) (0.297 ml, 0.297 mmol) in 1,4-dioxane (2 mL) was degassed and purged with N₂ (g) several times prior to heating in a sealed vial at 110° C. After 5 h, the crude reaction mixture was diluted with EtOAc (20 mL) and filtered through a pad of Celite™. The filtrate was washed with brine, dried over N₂SO₄, filtered, and concentrated in-vacuo. The crude product was purified by CombiFlash chromatography (24 g silica gel column, eluting with 0-15% methanol/dichloromethane) to afford 70 mg of desired product (91% yield). LCMS for C₄₀H₃₉FN₇O₇S (M+H)⁺: m/z=780.3; Found: 780.4.

### Step 10. 2-Fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzoic acid

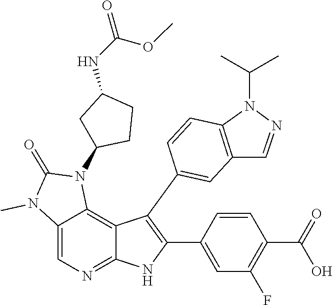 [SMILES: COC(=O)N[C@@H]1CC[C@@H](n2c(=O)n(C)c3cnc4[nH]c(-c5ccc(C(=O)O)c(F)c5)c(-c5ccc6c(cnn6C(C)C)c5)c4c32)C1]

A mixture of methyl 2-fluoro-4-(8-(1-isopropyl-1H-indazol-5-yl)-1-((1R,3R)-3-((methoxycarbonyl)amino)cyclopentyl)-3-methyl-2-oxo-6-(phenylsulfonyl)-1,2,3,6-tetrahydroimidazo[4,5-d]pyrrolo[2,3-b]pyridin-7-yl)benzoate (70 mg, 0.090 mmol) and lithium hydroxide monohydrate (38 mg, 0.90 mmol) in THF (1 mL), methanol (0.67 mL), and water (0.33 mL) was heated at 65° C. After 5 h, the reaction was diluted with H₂O (5 mL) and washed with ethyl acetate (2×5 mL) prior to neutralization with 1M HCl (aq) (0.89 ml, 0.89 mmol). The product partially precipitated out and was filtered off. The aqueous filtrate was extracted with DCM (4×5 mL), washed with brine, dried over N₂SO₄, filtered, and concentrated in-vacuo. The crude product was used in the next step without further purification. LCMS for C₃₃H₃₃FN₇O₅(M+H)⁺: m/z=626.2; Found: 626.3.

### Step 11. Methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

To a solution of 2-fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzoic acid (20 mg, 0.032 mmol) in DMF (0.6 ml) was added sequentially HATU (19 mg, 0.051 mmol), DIEA (0.028 mL, 0.16 mmol), and 2-(2,6-dioxopiperidin-3-yl)-4-(4-(piperidin-4-ylmethyl)piperazin-1-yl)isoindoline-1,3-dione, HCl (20 mg, 0.042 mmol, Sigma-Aldrich 923877). After stirring for 2 h, the reaction mixture was diluted with acetonitrile, filtered and then purified using prep-LCMS (XBridge C₁₈ column, eluting with a gradient of acetonitrile/water containing 0.1% TFA, at flow rate of 60 mL/min) to afford the desired product. LCMS for C₅₆H₆₀FN₁₂O₈(M+H)⁺: m/z=1047.5; Found: 1047.7.

### Examples 2-41

Examples 2-41 in Table 2A were prepared according to the procedures described in Example 1, using appropriately substituted starting materials. LC-MS data (calculated and found) for the compounds of Examples 2-41 are provided in Table 2B.

TABLE 2A

| Ex. | Name | Structure |
|---|---|---|
| 2 | methyl ((1R,3R)-3-(7-(4- ((3-((2-(2,6- dioxopiperidin-3-yl)-1,3- dioxoisoindolin-5- yl)amino)propyl) carbamoy 1)-3-fluorophenyl)-8-(3- fluoro-4-methoxyphenyl)- 3-methyl-2-oxo-3,6- dihydroimidazo[4,5- d]pyrrolo[2,3-b]pyridin- 1(2H)- yl)cyclopentyl) carbamate | 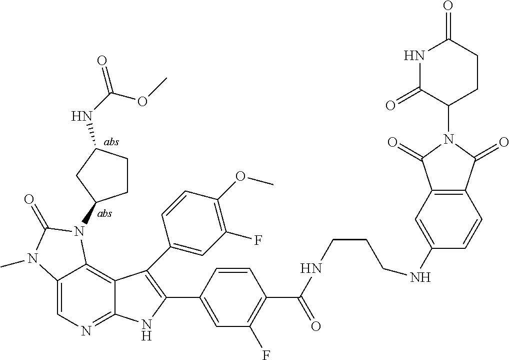 [SMILES: COC(=O)N[C@@H]1CC[C@@H](n2c(=O)n(C)c3cnc4[nH]c(-c5ccc(C(=O)NCCCNc6ccc7c(c6)C(=O)N(C6CCC(=O)NC6=O)C7=O)c(F)c5)c(-c5ccc(OC)c(F)c5)c4c32)C1] |
| 3 | methyl ((1R,3R)-3-(7-(4- ((4-((2-(2,6- dioxopiperidin-3-yl)-1,3- dioxoisoindolin-5- yl)amino)butyl) carbamoyl)- 3-fluorophenyl)-8-(3- fluoro-4-methoxyphenyl)- 3-methyl-2-oxo-3,6- dihydroimidazo[4,5- d]pyrrolo[2,3-b]pyridin- 1(2H)- yl)cyclopentyl) carbamate | 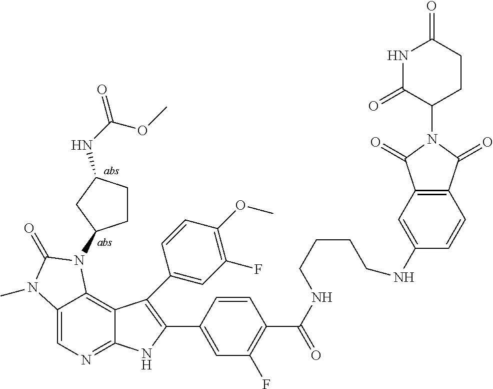 [SMILES: COC(=O)N[C@@H]1CC[C@@H](n2c(=O)n(C)c3cnc4[nH]c(-c5ccc(C(=O)NCCCCNc6ccc7c(c6)C(=O)N(C6CCC(=O)NC6=O)C7=O)c(F)c5)c(-c5ccc(OC)c(F)c5)c4c32)C1] |
| 4 | methyl ((1R,3R)-3-(7-(4- ((5-((2-(2,6- dioxopiperidin-3-yl)-1,3- dioxoisoindolin-5- yl)amino)pentyl) carbamoyl)- 3-fluorophenyl)-8-(3- fluoro-4-methoxyphenyl)- 3-methyl-2-oxo-3,6- dihydroimidazo[4,5- d]pyrrolo[2,3-b]pyridin- 1(2H)- yl)cyclopentyl) carbamate | 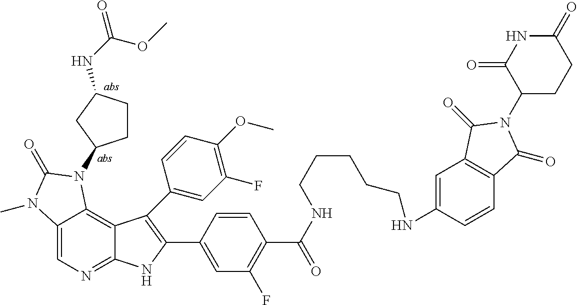 [SMILES: COC(=O)N[C@@H]1CC[C@@H](n2c(=O)n(C)c3cnc4[nH]c(-c5ccc(C(=O)NCCCCCNc6ccc7c(c6)C(=O)N(C6CCC(=O)NC6=O)C7=O)c(F)c5)c(-c5ccc(OC)c(F)c5)c4c32)C1] |
| 5 | methyl ((1R,3R)-3-(8-(1,1- dimethyl-1,3- dihydroisobenzofuran-5- yl)-7-(4-((4-((2-(2,6- dioxopiperidin-3-yl)-1,3- dioxoisoindolin-5- yl)amino)butyl) carbamoyl)- 3-fluorophenyl)-3-methyl- 2-oxo-3,6- dihydroimidazo[4,5- d]pyrrolo[2,3-b]pyridin- 1(2H)- yl)cyclopentyl) carbamate | 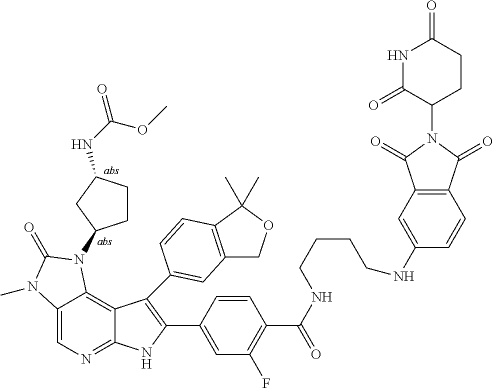 [SMILES: COC(=O)N[C@@H]1CC[C@@H](n2c(=O)n(C)c3cnc4[nH]c(-c5ccc(C(=O)NCCCCNc6ccc7c(c6)C(=O)N(C6CCC(=O)NC6=O)C7=O)c(F)c5)c(-c5ccc6c(c5)COC6(C)C)c4c32)C1] |
| 6 | methyl ((1R,3R)-3-(7-(4- ((2-(2-((2-(2,6- dioxopiperidin-3-yl)-1- oxoisoindolin-5-yl)amino)- 2- oxoethoxy)ethyl) carbamoyl)- 3-fluorophenyl)-8-(3- fluoro-4-methoxyphenyl)- 3-methyl-2-oxo-3,6- dihydroimidazo[4,5- d]pyrrolo[2,3-b]pyridin- 1(2H)- yl)cyclopentyl) carbamate | 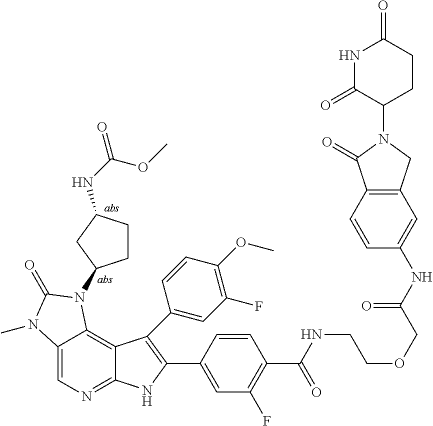 [SMILES: COC(=O)N[C@@H]1CC[C@@H](n2c(=O)n(C)c3cnc4[nH]c(-c5ccc(C(=O)NCCOCC(=O)Nc6ccc7c(c6)CN(C6CCC(=O)NC6=O)C7=O)c(F)c5)c(-c5ccc(OC)c(F)c5)c4c32)C1] |
| 7 | methyl ((1R,3R)-3-(7-(4- ((2-(2-((2-(2,6- dioxopiperidin-3-yl)-1,3- dioxoisoindolin-4- yl)amino)-2- oxoethoxy)ethyl) carbamoy 1)-3-fluorophenyl)-8-(3- fluoro-4-methoxyphenyl)- 3-methyl-2-oxo-3,6- dihydroimidazo[4,5- d]pyrrolo[2,3-b]pyridin- 1(2H)- yl)cyclopentyl) carbamate | 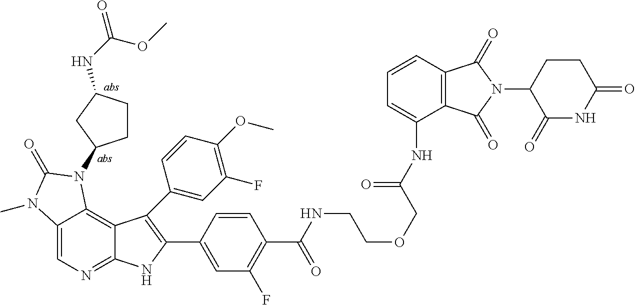 [SMILES: COC(=O)N[C@@H]1CC[C@@H](n2c(=O)n(C)c3cnc4[nH]c(-c5ccc(C(=O)NCCOCC(=O)Nc6cccc7c6C(=O)N(C6CCC(=O)NC6=O)C7=O)c(F)c5)c(-c5ccc(OC)c(F)c5)c4c32)C1] |
| 8 | methyl ((1R,3R)-3-(8-(1,1- dimethyl-1,3- dihydroisobenzofuran-5- yl)-7-(4-((3-((2-(2,6- dioxopiperidin-3-yl)-1,3- dioxoisoindolin-5- yl)amino)propyl) carbamoy 1)-3-fluorophenyl)-3- methyl-2-oxo-3,6- dihydroimidazo[4,5- d]pyrrolo[2,3-b]pyridin- 1(2H)- yl)cyclopentyl) carbamate | 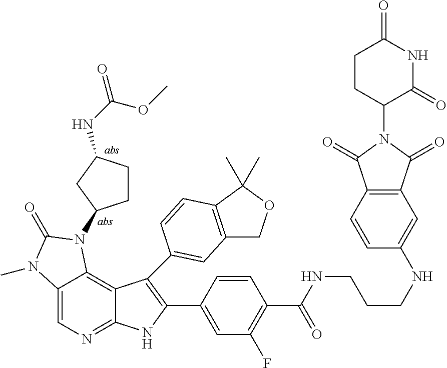 [SMILES: COC(=O)N[C@@H]1CC[C@@H](n2c(=O)n(C)c3cnc4[nH]c(-c5ccc(C(=O)NCCCNc6ccc7c(c6)C(=O)N(C6CCC(=O)NC6=O)C7=O)c(F)c5)c(-c5ccc6c(c5)COC6(C)C)c4c32)C1] |
| 9 | methyl ((1R,3R)-3-(8-(1,1- dimethyl-1,3- dihydroisobenzofuran-5- yl)-7-(4-((5-((2-(2,6- dioxopiperidin-3-yl)-1,3- dioxoisoindolin-5- yl)amino)pentyl) carbamoyl)- 3-fluorophenyl)-3- methyl-2-oxo-3,6- dihydroimidazo[4,5- d]pyrrolo[2,3-b]pyridin- 1(2H)- yl)cyclopentyl) carbamate | 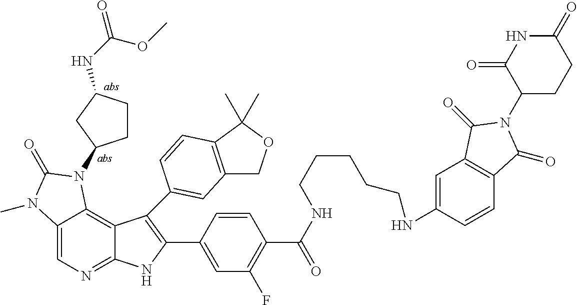 [SMILES: COC(=O)N[C@@H]1CC[C@@H](n2c(=O)n(C)c3cnc4[nH]c(-c5ccc(C(=O)NCCCCCNc6ccc7c(c6)C(=O)N(C6CCC(=O)NC6=O)C7=O)c(F)c5)c(-c5ccc6c(c5)COC6(C)C)c4c32)C1] |
| 10 | methyl ((1R,3R)-3-(8-(1,1- dimethyl-1,3- dihydroisobenzofuran-5- yl)-7-(4-((2-(2-((2-(2,6- dioxopiperidin-3-yl)-1- oxoisoindolin-5- yl)amino)-2- oxoethoxy)ethyl) carbamoyl)- 3-fluorophenyl)-3- methyl-2-oxo-3,6- dihydroimidazo[4,5- d]pyrrolo[2,3-b] pyridin-1(2H)- yl)cyclopentyl) carbamate | 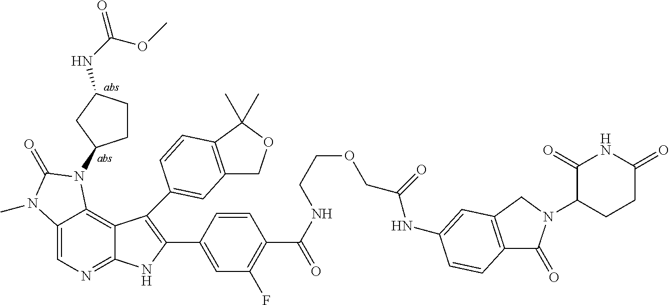 [SMILES: COC(=O)N[C@@H]1CC[C@@H](n2c(=O)n(C)c3cnc4[nH]c(-c5ccc(C(=O)NCCOCC(=O)Nc6ccc7c(c6)CN(C6CCC(=O)NC6=O)C7=O)c(F)c5)c(-c5ccc6c(c5)COC6(C)C)c4c32)C1] |
| 11 | methyl ((1R,3R)-3-(7-(4- ((4-((2-(2,6- dioxopiperidin-3-yl)-1,3- dioxoisoindolin-5- yl)amino)butyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 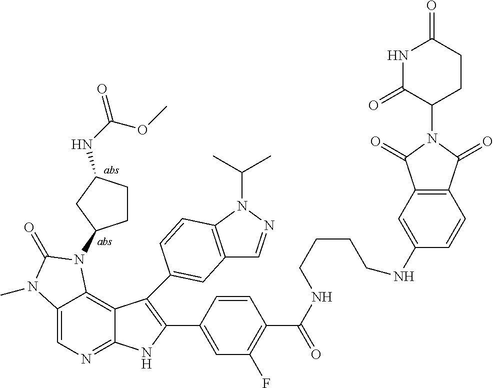 [SMILES: COC(=O)N[C@@H]1CC[C@@H](n2c(=O)n(C)c3cnc4[nH]c(-c5ccc(C(=O)NCCCCNc6ccc7c(c6)C(=O)N(C6CCC(=O)NC6=O)C7=O)c(F)c5)c(-c5ccc6c(cnn6C(C)C)c5)c4c32)C1] |
| 12 | methyl ((1R,3R)-3-(7-(4- ((5-((2-(2,6- dioxopiperidin-3-yl)-1,3- dioxoisoindolin-5- yl)amino)pentyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 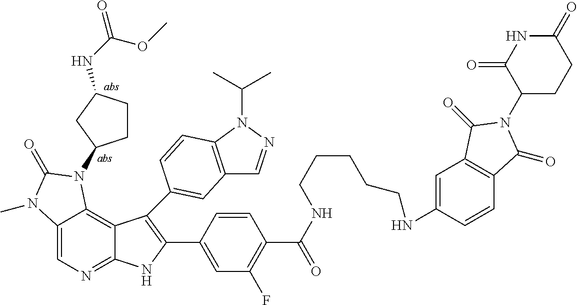 [SMILES: COC(=O)N[C@@H]1CC[C@@H](n2c(=O)n(C)c3cnc4[nH]c(-c5ccc(C(=O)NCCCCCNc6ccc7c(c6)C(=O)N(C6CCC(=O)NC6=O)C7=O)c(F)c5)c(-c5ccc6c(cnn6C(C)C)c5)c4c32)C1] |
| 13 | methyl ((1R,3R)-3-(7-(3- fluoro-4-((2-(2-(((S)-1- ((2S,4R)-4-hydroxy-2-((4- (4-methylthiazol-5- yl)benzyl) carbamoyl)pyrro lidin-1-yl)-3,3-dimethyl-1- oxobutan-2-yl)amino)-2- oxoethoxy)ethyl) carbamoy 1)phenyl)-8-(1-isopropyl- 1H-indazol-5-yl)-3-methyl- 2-oxo-3,6- dihydroimidazo[4,5- d]pyrrolo[2,3-b]pyridin- 1(2H)- yl)cyclopentyl) carbamate | 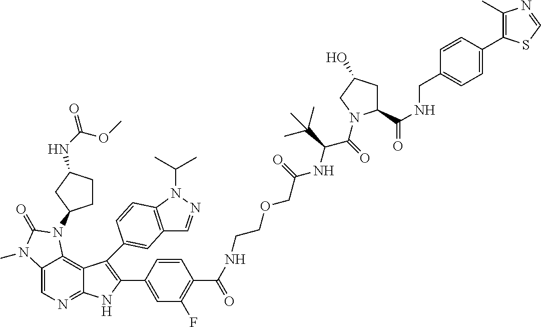 [SMILES: COC(=O)N[C@@H]1CC[C@@H](n2c(=O)n(C)c3cnc4[nH]c(-c5ccc(C(=O)NCCOCC(=O)N[C@H](C(=O)N6C[C@H](O)C[C@H]6C(=O)NCc6ccc(-c7scnc7C)cc6)C(C)(C)C)c(F)c5)c(-c5ccc6c(cnn6C(C)C)c5)c4c32)C1] |
| 14 | methyl ((1R,3R)-3-(7-(3- fluoro-4-((2-(2-(2-(((S)-1- ((2S,4R)-4-hydroxy-2-((4- (4-methylthiazol-5- yl)benzyl) carbamoyl) pyrrolidin- 1-yl)-3,3-dimethyl-1- oxobutan-2-yl)amino)-2- oxoethoxy)ethoxy)ethyl) carbamoyl)phenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 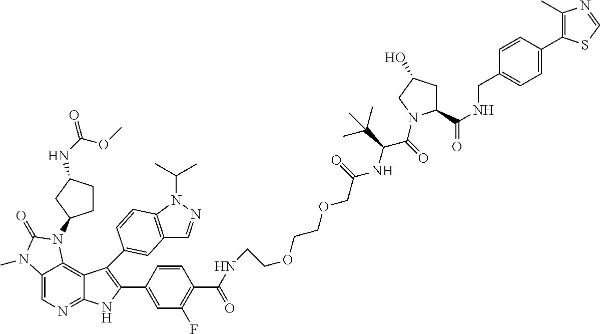 [SMILES: COC(=O)N[C@@H]1CC[C@@H](n2c(=O)n(C)c3cnc4[nH]c(-c5ccc(C(=O)NCCOCCOCC(=O)N[C@H](C(=O)N6C[C@H](O)C[C@H]6C(=O)NCc6ccc(-c7scnc7C)cc6)C(C)(C)C)c(F)c5)c(-c5ccc6c(cnn6C(C)C)c5)c4c32)C1] |
| 15 | methyl ((1R,3R)-3-(7-(4- (4-(2-(3-((2-(2,6- dioxopiperidin-3-yl)-1- oxoisoindolin-5-yl)amino)- 3-oxopropoxy)ethyl) piperazine- 1-carbonyl)-3- fluorophenyl)-8-(3-fluoro- 4-methoxyphenyl)-3- methyl-2-oxo-3,6- dihydroimidazo[4,5- d]pyrrolo[2,3-b]pyridin- 1(2H)- yl)cyclopentyl) carbamate | 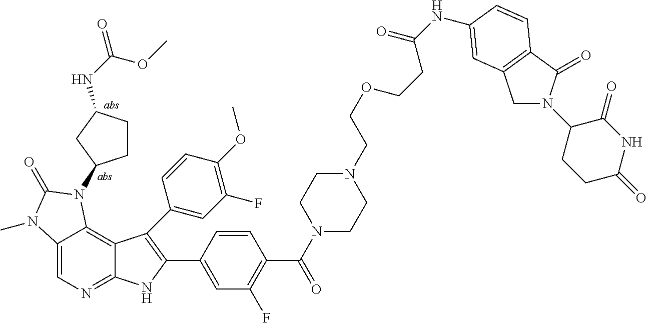 [SMILES: COC(=O)N[C@@H]1CC[C@@H](n2c(=O)n(C)c3cnc4[nH]c(-c5ccc(C(=O)N6CCN(CCOCCC(=O)Nc7ccc8c(c7)CN(C7CCC(=O)NC7=O)C8=O)CC6)c(F)c5)c(-c5ccc(OC)c(F)c5)c4c32)C1] |
| 16 | methyl ((1R,3R)-3-(7-(4- (4-(4-((2-(2,6- dioxopiperidin-3-yl)-1- oxoisoindolin-5- yl) carbamoyl)benzyl) piperazine-1-carbonyl)-3- fluorophenyl)-8-(3-fluoro- 4-methoxyphenyl)-3- methyl-2-oxo-3,6- dihydroimidazo[4,5- d]pyrrolo[2,3-b]pyridin- 1(2H)- yl)cyclopentyl) carbamate | 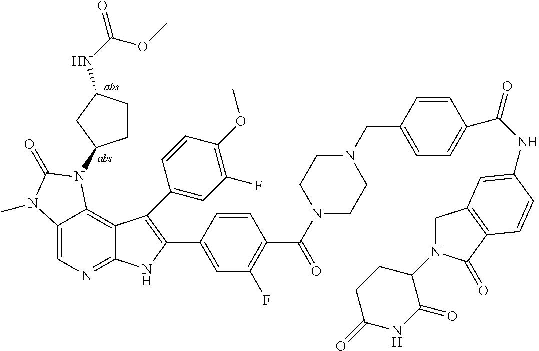 [SMILES: COC(=O)N[C@@H]1CC[C@@H](n2c(=O)n(C)c3cnc4[nH]c(-c5ccc(C(=O)N6CCN(Cc7ccc(C(=O)Nc8ccc9c(c8)CN(C8CCC(=O)NC8=O)C9=O)cc7)CC6)c(F)c5)c(-c5ccc(OC)c(F)c5)c4c32)C1] |
| 17 | methyl ((1R,3R)-3-(7-(4- ((2-(2-((2-(2,6- dioxopiperidin-3-yl)-1- oxoisoindolin-5-yl)amino)- 2-oxoethoxy)ethyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 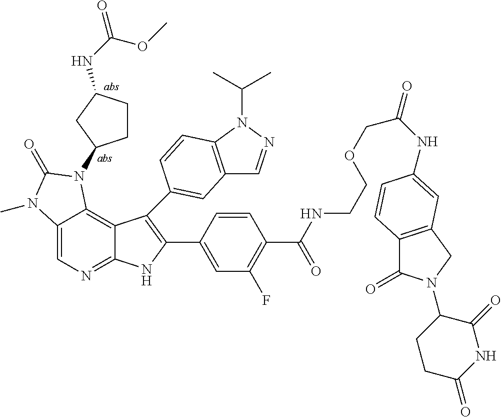 [SMILES: COC(=O)N[C@@H]1CC[C@@H](n2c(=O)n(C)c3cnc4[nH]c(-c5ccc(C(=O)NCCOCC(=O)Nc6ccc7c(c6)CN(C6CCC(=O)NC6=O)C7=O)c(F)c5)c(-c5ccc6c(cnn6C(C)C)c5)c4c32)C1] |
| 18 | methyl ((1R,3R)-3-(7-(4- (4-(4-((2-(2,6- dioxopiperidin-3-yl)-1- oxoisoindolin-5- yl) carbamoyl)benzyl) piperazine-1-carbonyl)-3- fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 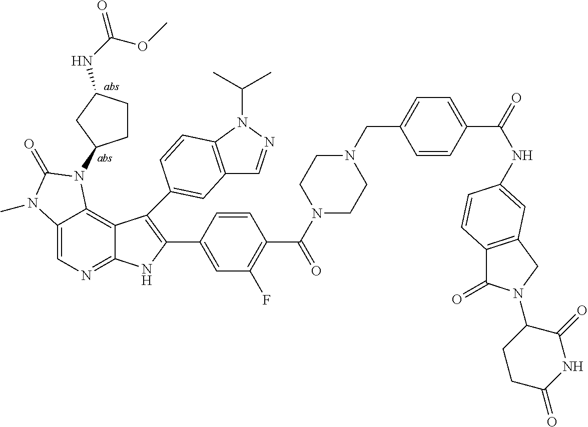 [SMILES: COC(=O)N[C@@H]1CC[C@@H](n2c(=O)n(C)c3cnc4[nH]c(-c5ccc(C(=O)N6CCN(Cc7ccc(C(=O)Nc8ccc9c(c8)CN(C8CCC(=O)NC8=O)C9=O)cc7)CC6)c(F)c5)c(-c5ccc6c(cnn6C(C)C)c5)c4c32)C1] |
| 19 | methyl ((1R,3R)-3-(7-(4- ((7-((2-(2,6- dioxopiperidin-3-yl)-1- oxoisoindolin-5-yl)amino)- 7-oxoheptyl) carbamoyl)-3- fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 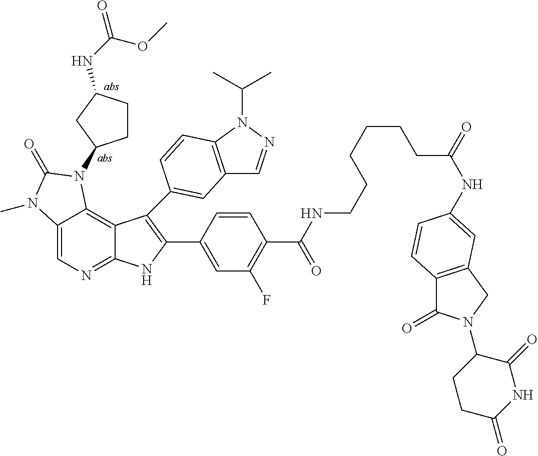 [SMILES: COC(=O)N[C@@H]1CC[C@@H](n2c(=O)n(C)c3cnc4[nH]c(-c5ccc(C(=O)NCCCCCCC(=O)Nc6ccc7c(c6)CN(C6CCC(=O)NC6=O)C7=O)c(F)c5)c(-c5ccc6c(cnn6C(C)C)c5)c4c32)C1] |
| 20 | methyl ((1R,3R)-3-(7-(4- (4-(4-((2-(2,6- dioxopiperidin-3-yl)-1,3- dioxoi soindolin-4- yl) carbamoyl)benzyl) piperazine-1-carbonyl)-3- fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 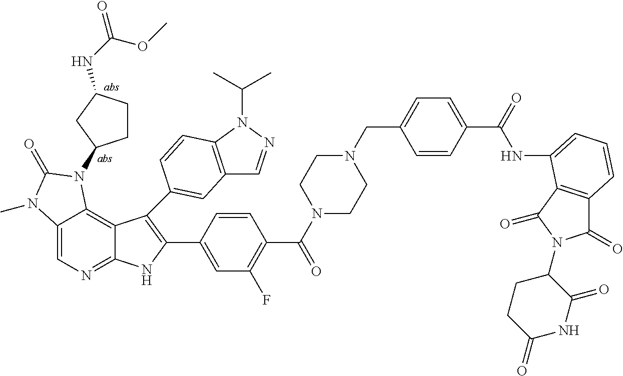 [SMILES: COC(=O)N[C@@H]1CC[C@@H](n2c(=O)n(C)c3cnc4[nH]c(-c5ccc(C(=O)N6CCN(Cc7ccc(C(=O)Nc8cccc9c8C(=O)N(C8CCC(=O)NC8=O)C9=O)cc7)CC6)c(F)c5)c(-c5ccc6c(cnn6C(C)C)c5)c4c32)C1] |
| 21 | methyl ((1R,3R)-3-(7-(4- ((2-(2-((2-(2,6- dioxopiperidin-3-yl)-1,3- dioxoisoindolin-4- yl)amino)-2- oxoethoxy)ethyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 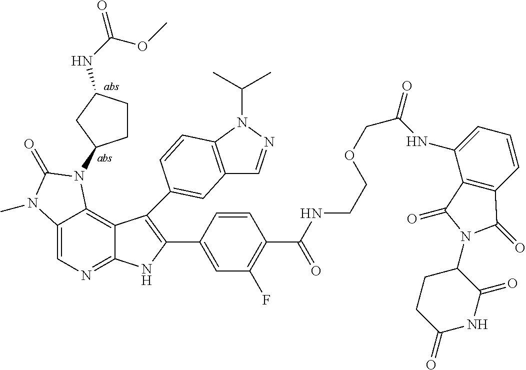 [SMILES: COC(=O)N[C@@H]1CC[C@@H](n2c(=O)n(C)c3cnc4[nH]c(-c5ccc(C(=O)NCCOCC(=O)Nc6cccc7c6C(=O)N(C6CCC(=O)NC6=O)C7=O)c(F)c5)c(-c5ccc6c(cnn6C(C)C)c5)c4c32)C1] |
| 22 | methyl ((1R,3R)-3-(7-(4- (4-(4-(3-(2-((2-(2,6- dioxopiperidin-3-yl)-1,3- dioxoisoindolin-4- yl)amino)-2-oxoethoxy)- 2,2- difluoropropoxy)butyl) piperazine-1-carbonyl)-3- fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 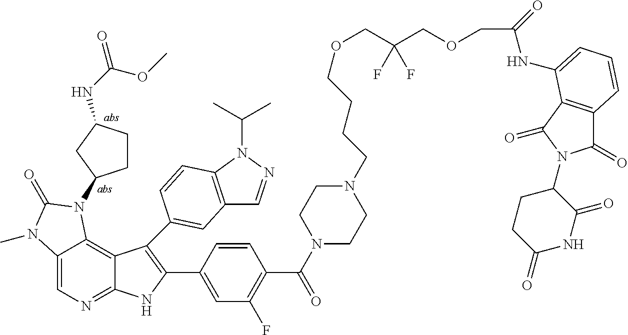 [SMILES: COC(=O)N[C@@H]1CC[C@@H](n2c(=O)n(C)c3cnc4[nH]c(-c5ccc(C(=O)N6CCN(CCCCOCC(F)(F)COCC(=O)Nc7cccc8c7C(=O)N(C7CCC(=O)NC7=O)C8=O)CC6)c(F)c5)c(-c5ccc6c(cnn6C(C)C)c5)c4c32)C1] |
| 23 | methyl ((1R,3R)-3-(7-(4- ((2-(2-((2-(2,6- dioxopiperidin-3-yl)-3- oxoisoindolin-4-yl)amino)- 2-oxoethoxy)ethyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 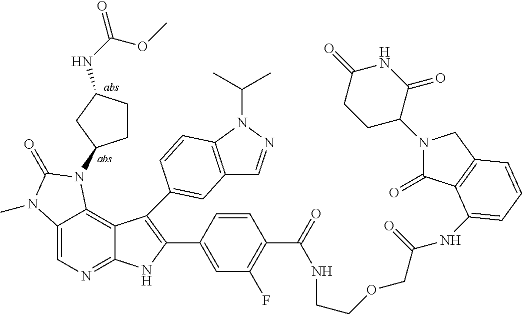 [SMILES: COC(=O)N[C@@H]1CC[C@@H](n2c(=O)n(C)c3cnc4[nH]c(-c5ccc(C(=O)NCCOCC(=O)Nc6cccc7c6C(=O)N(C6CCC(=O)NC6=O)C7)c(F)c5)c(-c5ccc6c(cnn6C(C)C)c5)c4c32)C1] |
| 24 | methyl ((1R,3R)-3-(7-(4- ((2-(2-((2-(2,6- dioxopiperidin-3-yl)-1- oxoisoindolin-4-yl)amino)- 2-oxoethoxy)ethyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 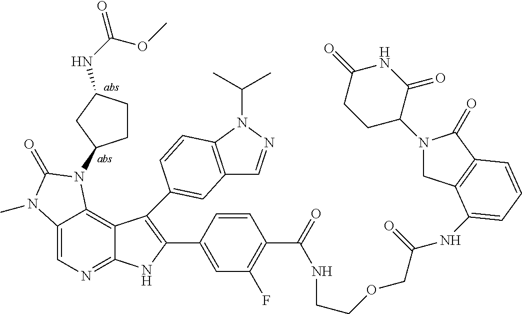 [SMILES: COC(=O)N[C@@H]1CC[C@@H](n2c(=O)n(C)c3cnc4[nH]c(-c5ccc(C(=O)NCCOCC(=O)Nc6cccc7c6CN(C6CCC(=O)NC6=O)C7=O)c(F)c5)c(-c5ccc6c(cnn6C(C)C)c5)c4c32)C1] |
| 25 | methyl ((1R,3R)-3-(7-(4- ((2-(2-((2-(2,6- dioxopiperidin-3-yl)-3- oxoisoindolin-5-yl)amino)- 2-oxoethoxy)ethyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 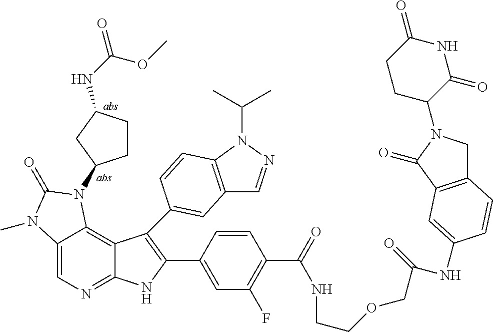 [SMILES: COC(=O)N[C@@H]1CC[C@@H](n2c(=O)n(C)c3cnc4[nH]c(-c5ccc(C(=O)NCCOCC(=O)Nc6ccc7c(c6)C(=O)N(C6CCC(=O)NC6=O)C7)c(F)c5)c(-c5ccc6c(cnn6C(C)C)c5)c4c32)C1] |
| 26 | methyl ((1R,3R)-3-(7-(4- ((2-(2-((2-(2,6- dioxopiperidin-3-yl)-1,3- dioxoisoindolin-5- yl)amino)-2- oxoethoxy)ethyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 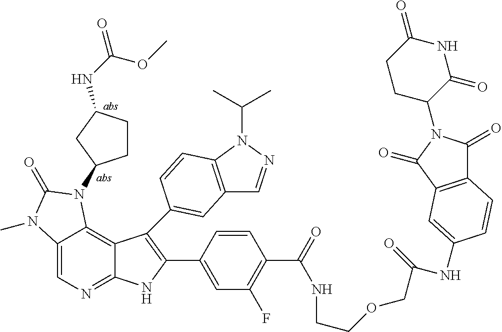 [SMILES: COC(=O)N[C@@H]1CC[C@@H](n2c(=O)n(C)c3cnc4[nH]c(-c5ccc(C(=O)NCCOCC(=O)Nc6ccc7c(c6)C(=O)N(C6CCC(=O)NC6=O)C7=O)c(F)c5)c(-c5ccc6c(cnn6C(C)C)c5)c4c32)C1] |
| 27 | methyl ((1R,3R)-3-(7-(4- ((2-(2-((2-(2,6- dioxopiperidin-3-yl)-1- oxoisoindolin-5-yl)amino)- 2- oxoethoxy)ethyl)(methyl) carbamoyl)-3- fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 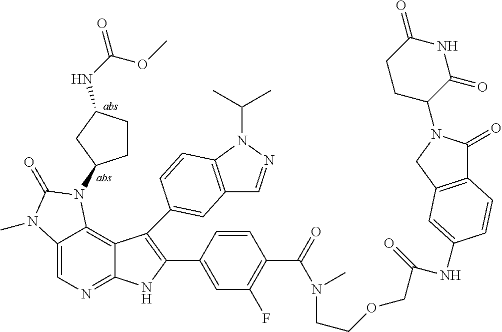 [SMILES: COC(=O)N[C@@H]1CC[C@@H](n2c(=O)n(C)c3cnc4[nH]c(-c5ccc(C(=O)N(C)CCOCC(=O)Nc6ccc7c(c6)CN(C6CCC(=O)NC6=O)C7=O)c(F)c5)c(-c5ccc6c(cnn6C(C)C)c5)c4c32)C1] |
| 28 | methyl ((1R,3R)-3-(7-(4- (4-((4-(2-(2,6- dioxopiperidin-3-yl)-1,3- dioxoisoindolin-4- yl)piperazin-1- yl)methyl)piperidine-1- carbonyl)-3- methoxyphenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 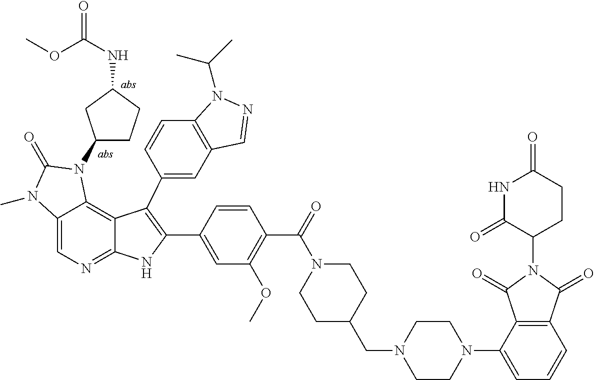 [SMILES: COC(=O)N[C@@H]1CC[C@@H](n2c(=O)n(C)c3cnc4[nH]c(-c5ccc(C(=O)N6CCC(CN7CCN(c8cccc9c8C(=O)N(C8CCC(=O)NC8=O)C9=O)CC7)CC6)c(OC)c5)c(-c5ccc6c(cnn6C(C)C)c5)c4c32)C1] |
| 29 | methyl ((1R,3R)-3-(7-(4- ((2-(2-(((S)-1-(((S)-1- cyclohexyl-2-((S)-2-(((S)- 1-(ethylamino)-1-oxo-3- phenylpropan-2-yl) carbamoyl)pyrrolidin-1- yl)-2-oxoethyl)amino)-1- oxopropan-2- yl)amino)ethoxy)ethyl) carbamoyl)- 3-fluorophenyl)-8- (1-isopropyl-1H-indazol-5- yl)-3-methyl-2-oxo-3,6- dihydroimidazo[4,5- d]pyrrolo[2,3-b]pyridin- 1(2H)-yl)cyclopentyl) carbamate | 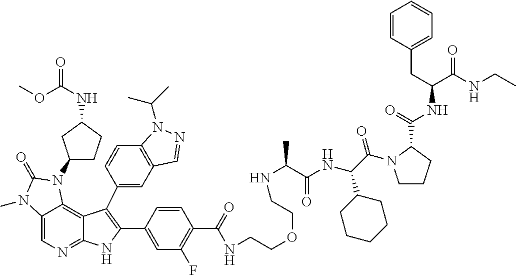 [SMILES: CCNC(=O)[C@H](Cc1ccccc1)NC(=O)[C@@H]1CCCN1C(=O)[C@@H](NC(=O)[C@H](C)NCCOCCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1F)C1CCCCC1] |
| 30 | methyl ((1R,3R)-3-(7-(4- ((6-(((S)-1-(((S)-1- cyclohexyl-2-((S)-2-(((S)- 1-(ethylamino)-1-oxo-3- phenylpropan-2- yl) carbamoyl)pyrrolidin-1- yl)-2-oxoethyl)amino)-1- oxopropan-2- yl)amino)hexyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 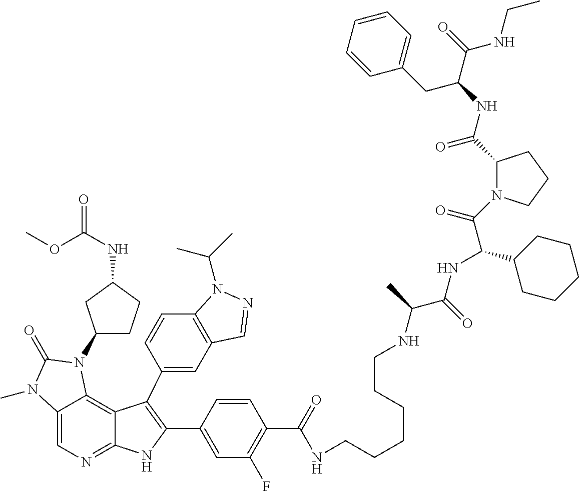 [SMILES: CCNC(=O)[C@H](Cc1ccccc1)NC(=O)[C@@H]1CCCN1C(=O)[C@@H](NC(=O)[C@H](C)NCCCCCCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1F)C1CCCCC1] |
| 31 | methyl ((1R,3R)-3-(7-(4- ((2-(2-(((S)-1-(((S)-1- cyclohexyl-2-((S)-2-(((S)- 1-(ethylamino)-3-(4- fluorophenyl)-1- oxopropan-2- yl) carbamoyl)pyrrolidin-1- yl)-2-oxoethyl)amino)-1- oxopropan-2- yl)amino)ethoxy)ethyl) carbamoyl)-3- fluorophenyl)-8- (1-isopropyl-1H-indazol-5- yl)-3-methyl-2-oxo-3,6- dihydroimidazo[4,5- d]pyrrolo[2,3-b]pyridin- 1(2H)-yl)cyclopentyl) carbamate | 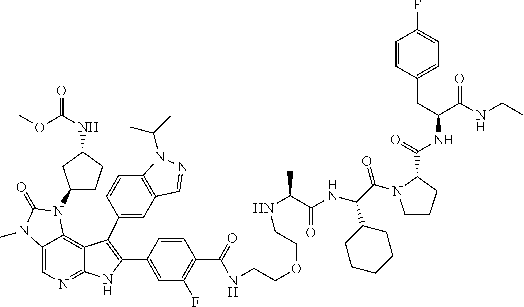 [SMILES: CCNC(=O)[C@H](Cc1ccc(F)cc1)NC(=O)[C@@H]1CCCN1C(=O)[C@@H](NC(=O)[C@H](C)NCCOCCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1F)C1CCCCC1] |
| 32 | methyl ((1R,3R)-3-(7-(4- (4-((4-(2-(2,6- dioxopiperidin-3-yl)-1,3- dioxoisoindolin-5- yl)piperazin-1- yl)methyl)piperidine-1- carbonyl)-3-fluorophenyl)- 8-(1-isopropyl-1H-indazol- 5-yl)-3-methyl-2-oxo-3,6- dihydroimidazo[4,5- d]pyrrolo[2,3-b]pyridin- 1(2H)- yl)cyclopentyl) carbamate | 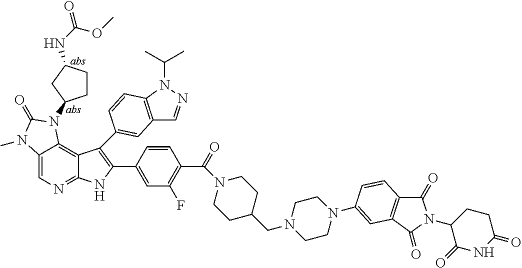 [SMILES: COC(=O)N[C@@H]1CC[C@@H](n2c(=O)n(C)c3cnc4[nH]c(-c5ccc(C(=O)N6CCC(CN7CCN(c8ccc9c(c8)C(=O)N(C8CCC(=O)NC8=O)C9=O)CC7)CC6)c(F)c5)c(-c5ccc6c(cnn6C(C)C)c5)c4c32)C1] |
| 33 | methyl ((1R,3R)-3-(7-(4- (4-((4-(2-(2,6- dioxopiperidin-3-yl)-1,3- dioxoisoindolin-4- yl)piperazin-1- yl)methyl)piperidine-1- carbonyl)phenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 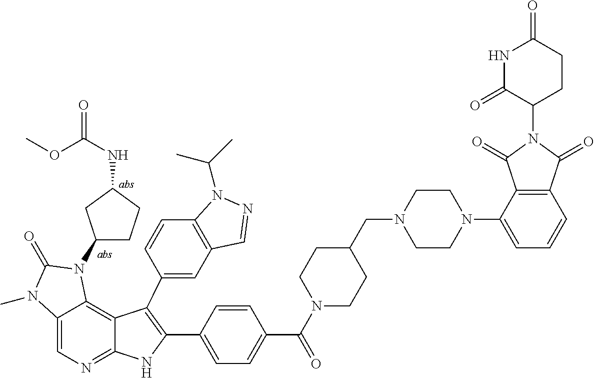 [SMILES: COC(=O)N[C@@H]1CC[C@@H](n2c(=O)n(C)c3cnc4[nH]c(-c5ccc(C(=O)N6CCC(CN7CCN(c8cccc9c8C(=O)N(C8CCC(=O)NC8=O)C9=O)CC7)CC6)cc5)c(-c5ccc6c(cnn6C(C)C)c5)c4c32)C1] |
| 34 | methyl ((1R,3R)-3-(7-(4- (4-((4-(2-(2,6- dioxopiperidin-3-yl)-1,3- dioxoisoindolin-5- yl)piperazin-1- yl)methyl)piperidine-1- carbonyl)phenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 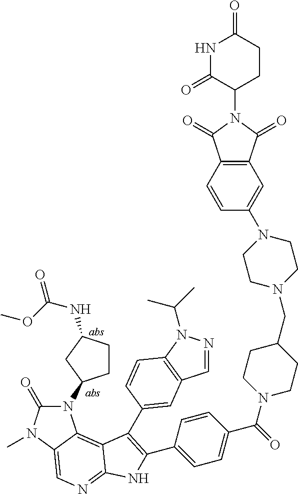 [SMILES: COC(=O)N[C@@H]1CC[C@@H](n2c(=O)n(C)c3cnc4[nH]c(-c5ccc(C(=O)N6CCC(CN7CCN(c8ccc9c(c8)C(=O)N(C8CCC(=O)NC8=O)C9=O)CC7)CC6)cc5)c(-c5ccc6c(cnn6C(C)C)c5)c4c32)C1] |
| 35 | methyl ((1R,3R)-3-(7-(4- (4-((4-(2-(2,6- dioxopiperidin-3-yl)-1- oxoisoindolin-5- yl)piperazin-1- yl)methyl)piperidine-1- carbonyl)phenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 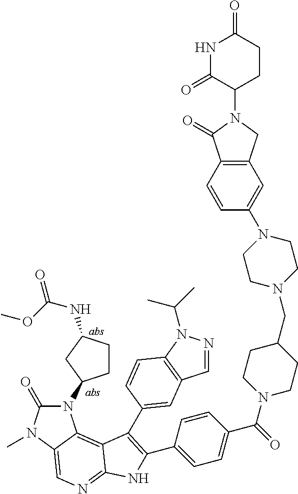 [SMILES: COC(=O)N[C@@H]1CC[C@@H](n2c(=O)n(C)c3cnc4[nH]c(-c5ccc(C(=O)N6CCC(CN7CCN(c8ccc9c(c8)CN(C8CCC(=O)NC8=O)C9=O)CC7)CC6)cc5)c(-c5ccc6c(cnn6C(C)C)c5)c4c32)C1] |
| 36 | methyl ((1R,3R)-3-(7-(4- (4-((4-(2-(2,6- dioxopiperidin-3-yl)-1- oxoisoindolin-5- yl)piperazin-1- yl)methyl)piperidine-1- carbonyl)-3-fluorophenyl)- 8-(1-isopropyl-1H-indazol- 5-yl)-3-methyl-2-oxo-3,6- dihydroimidazo[4,5- d]pyrrolo[2,3-b]pyridin- 1(2H)- yl)cyclopentyl) carbamate | 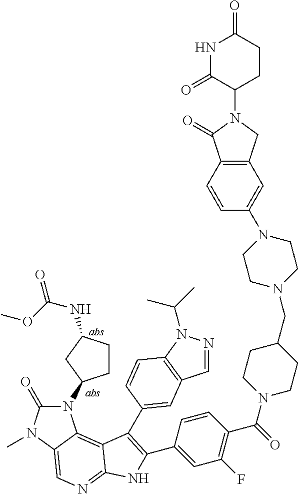 [SMILES: COC(=O)N[C@@H]1CC[C@@H](n2c(=O)n(C)c3cnc4[nH]c(-c5ccc(C(=O)N6CCC(CN7CCN(c8ccc9c(c8)CN(C8CCC(=O)NC8=O)C9=O)CC7)CC6)c(F)c5)c(-c5ccc6c(cnn6C(C)C)c5)c4c32)C1] |
| 37 | methyl ((1R,3R)-3-(7-(4- (4-(4-(2-(2,6- dioxopiperidin-3-yl)-1,3- dioxoisoindolin-4- yl)piperazine-1- carbonyl)piperidine-1- carbonyl)phenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 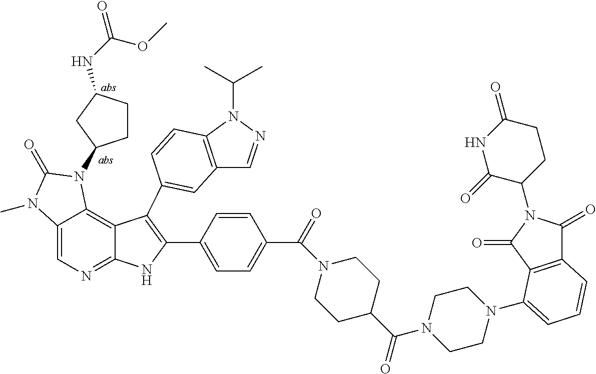 [SMILES: COC(=O)N[C@@H]1CC[C@@H](n2c(=O)n(C)c3cnc4[nH]c(-c5ccc(C(=O)N6CCC(C(=O)N7CCN(c8cccc9c8C(=O)N(C8CCC(=O)NC8=O)C9=O)CC7)CC6)cc5)c(-c5ccc6c(cnn6C(C)C)c5)c4c32)C1] |
| 38 | methyl ((1R,3R)-3-(7-(4- (4-((1-(2-(2,6- dioxopiperidin-3-yl)-1,3- dioxoisoindolin-4- yl)piperidin-4- yl)methyl)piperazine-1- carbonyl)phenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 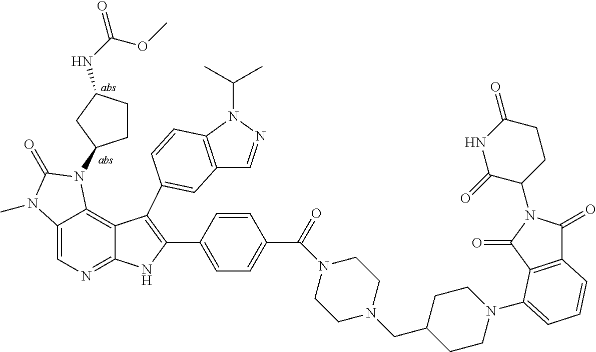 [SMILES: COC(=O)N[C@@H]1CC[C@@H](n2c(=O)n(C)c3cnc4[nH]c(-c5ccc(C(=O)N6CCN(CC7CCN(c8cccc9c8C(=O)N(C8CCC(=O)NC8=O)C9=O)CC7)CC6)cc5)c(-c5ccc6c(cnn6C(C)C)c5)c4c32)C1] |
| 39 | methyl ((1R,3R)-3-(7-(4- ((4-((2-(2,6- dioxopiperidin-3-yl)-1,3- dioxoisoindolin-4- yl)amino)butyl) carbamoyl)- 3-fluorophenyl)-8-(1- isopropyl-1H-indazol-5- yl)-3-methyl-2-oxo-3,6- dihydroimidazo[4,5- d]pyrrolo[2,3-b]pyridin- 1(2H)- yl)cyclopentyl) carbamate | 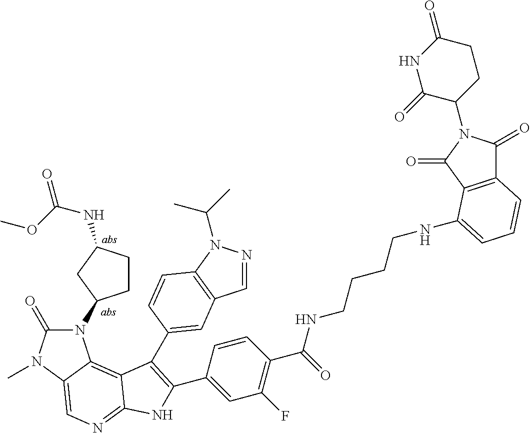 [SMILES: COC(=O)N[C@@H]1CC[C@@H](n2c(=O)n(C)c3cnc4[nH]c(-c5ccc(C(=O)NCCCCNc6cccc7c6C(=O)N(C6CCC(=O)NC6=O)C7=O)c(F)c5)c(-c5ccc6c(cnn6C(C)C)c5)c4c32)C1] |
| 40 | methyl ((1R,3R)-3-(7-(1- (2-(4-((4-(2-(2,6- dioxopiperidin-3-yl)-1,3- dioxoisoindolin-4- yl)piperazin-1- yl)methyl)piperidin-1-yl)- 2-oxoethyl)-1H-pyrazol-4- yl)-8-(1-isopropyl-1H- indazol-5-yl)-3-methyl-2- oxo-3,6- dihydroimidazo[4,5- d]pyrrolo[2,3-b]pyridin- 1(2H)- yl)cyclopentyl) carbamate | 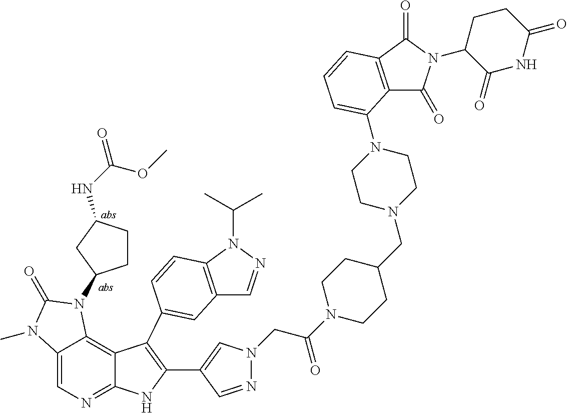 [SMILES: COC(=O)N[C@@H]1CC[C@@H](n2c(=O)n(C)c3cnc4[nH]c(-c5cnn(CC(=O)N6CCC(CN7CCN(c8cccc9c8C(=O)N(C8CCC(=O)NC8=O)C9=O)CC7)CC6)c5)c(-c5ccc6c(cnn6C(C)C)c5)c4c32)C1] |
| 41 | methyl ((1R,3R)-3-(7-(1- (2-(4-((4-(2-(2,6- dioxopiperidin-3-yl)-1- oxoisoindolin-5- yl)piperazin-1- yl)methyl)piperidin-1-yl)- 2-oxoethyl)-1H-pyrazol-4- yl)-8-(1-isopropyl-1H- indazol-5-yl)-3-methyl-2- oxo-3,6- dihydroimidazo[4,5- d]pyrrolo[2,3-b]pyridin- 1(2H)- yl)cyclopentyl) carbamate | 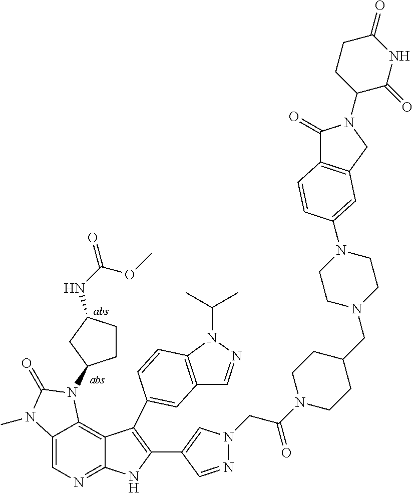 [SMILES: COC(=O)N[C@@H]1CC[C@@H](n2c(=O)n(C)c3cnc4[nH]c(-c5cnn(CC(=O)N6CCC(CN7CCN(c8ccc9c(c8)CN(C8CCC(=O)NC8=O)C9=O)CC7)CC6)c5)c(-c5ccc6c(cnn6C(C)C)c5)c4c32)C1] |

TABLE 2B

| | M + 1H | M + 1H |
|---|---|---|
| Example | (calc) | found |
| 2 | 904.3 | 904.3 |
| 3 | 918.3 | 918.3 |
| 4 | 932.3 | 932.3 |
| 5 | 940.4 | 940.4 |
| 6 | 934.3 | 934.4 |
| 7 | 948.3 | 948.3 |
| 8 | 926.4 | 926.4 |
| 9 | 954.4 | 954.4 |
| 10 | 956.4 | 956.4 |
| 11 | 952.4 | 952.5 |
| 12 | 966.4 | 966.5 |
| 13 | 1139.5 | 1139.5 |
| 14 | 1183.5 | 1183.6 |
| 15 | 1017.4 | 1017.4 |
| 16 | 1035.4 | 1035.4 |
| 17 | 968.4 | 968.4 |
| 18 | 1069.4 | 1069.4 |
| 19 | 994.4 | 994.5 |
| 20 | 1083.4 | 1083.4 |
| 21 | 982.4 | 982.4 |
| 22 | 1173.5 | 1173.4 |
| 23 | 968.4 | 968.6 |
| 24 | 968.4 | 968.6 |
| 25 | 968.4 | 968.6 |
| 26 | 982.4 | 982.5 |
| 27 | 982.4 | 982.4 |
| 28 | 1059.5 | 1059.4 |
| 29 | 1194.6 | 1194.6 |
| 30 | 1206.7 | 1206.7 |
| 31 | 1212.6 | 1212.6 |
| 32 | 1047.5 | 1047.4 |
| 33 | 1029.5 | 1029.5 |
| 34 | 1029.5 | 1029.6 |
| 35 | 1015.5 | 1015.5 |
| 36 | 1033.5 | 1033.6 |
| 37 | 1043.4 | 1043.5 |
| 38 | 1029.5 | 1029.5 |
| 39 | 952.4 | 952.4 |
| 40 | 1033.5 | 1033.5 |
| 41 | 1019.5 | 1019.5 |

### Example 42. Methyl((1R,3R)-3-(7-(4-((4-((1-(2-(2,6-dioxopiperidin-3-yl)-1,3-dioxoisoindolin-4-yl)piperidin-4-yl)methyl)piperazin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

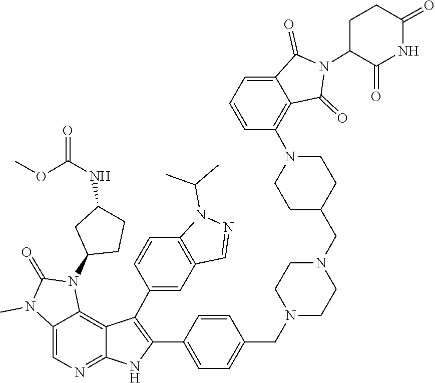 [SMILES: COC(=O)N[C@@H]1CC[C@@H](n2c(=O)n(C)c3cnc4[nH]c(-c5ccc(CN6CCN(CC7CCN(c8cccc9c8C(=O)N(C8CCC(=O)NC8=O)C9=O)CC7)CC6)cc5)c(-c5ccc6c(cnn6C(C)C)c5)c4c32)C1]

### Step 1. Methyl((1R,3R)-3-(7-(4-formylphenyl)-8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate

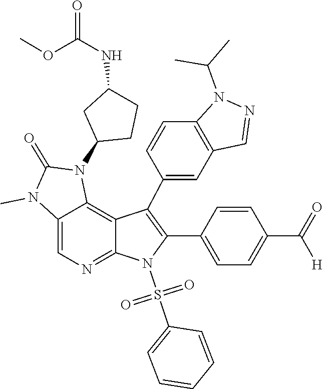 [SMILES: COC(=O)N[C@@H]1CC[C@@H](n2c(=O)n(C)c3cnc4c(c(-c5ccc6c(cnn6C(C)C)c5)c(-c5ccc(C=O)cc5)n4S(=O)(=O)c4ccccc4)c32)C1]

A procedure analogous to the one described in Example 1 steps 1-9 was used with the exception that 4-formylphenylboronic acid was used as the coupling partner in step 9 to prepare the title compound. LCMS for C₃₉H₃₈N₇O₆S (M+H)⁺: m/z=732.3; Found: 732.4.

### Step 2. Methyl((1R,3R)-3-(7-(4-((4-((1-(2-(2,6-dioxopiperidin-3-yl)-1,3-dioxoisoindolin-4-yl)piperidin-4-yl)methyl)piperazin-1-yl)methyl)phenyl)-8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate

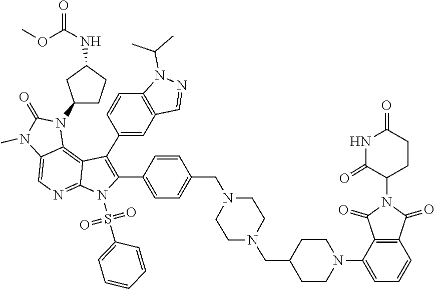 [SMILES: COC(=O)N[C@@H]1CC[C@@H](n2c(=O)n(C)c3cnc4c(c(-c5ccc6c(cnn6C(C)C)c5)c(-c5ccc(CN6CCN(CC7CCN(c8cccc9c8C(=O)N(C8CCC(=O)NC8=O)C9=O)CC7)CC6)cc5)n4S(=O)(=O)c4ccccc4)c32)C1]

To a solution of methyl((1R,3R)-3-(7-(4-formylphenyl)-8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate (9.0 mg, 0.012 mmol) and 2-(2,6-dioxopiperidin-3-yl)-4-(4-(piperidin-4-ylmethyl)piperazin-1-yl)isoindoline-1,3-dione, HCl (12 mg, 0.025 mmol, Sigma-Aldrich 923931) in DCE (0.6 ml) was added triethylamine (3.4 μl, 0.025 mmol) to neutralize the HCl salt followed by sodium cyanoborohydride (2.3 mg, 0.037 mmol). The reaction was stirred at ambient temperature overnight. The crude reaction mixture was purified by CombiFlash chromatography (24 g silica gel column, eluting with 0-15% MeOH/DCM) to afford the desired product (9.0 mg, 63% yield). LCMS for C₆₂H₆₇N₁₂O₉S (M+H)⁺: m/z=1155.5; Found: 1155.5.

### Step 3. Methyl((1R,3R)-3-(7-(4-((4-((1-(2-(2,6-dioxopiperidin-3-yl)-1,3-dioxoisoindolin-4-yl)piperidin-4-yl)methyl)piperazin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

To a solution of methyl((1R,3R)-3-(7-(4-((4-((1-(2-(2,6-dioxopiperidin-3-yl)-1,3-dioxoisoindolin-4-yl)piperidin-4-yl)methyl)piperazin-1-yl)methyl)phenyl)-8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate (11 mg, 9.5 μmol) in anhydrous THF (0.5 ml) was added TBAF (1.0 M in THF, 0.095 ml, 0.095 mmol) and the resulting solution was stirred at 50° C. in a sealed vial. After stirring overnight, the reaction mixture was diluted with acetonitrile, filtered, and then purified using prep-LCMS (XBridge C₁₈ column, eluting with a gradient of acetonitrile/water containing 0.1% TFA, at flow rate of 60 mL/min) to afford the desired product. LCMS for C₅₆H₆₃N₁₂O₇ (M+H)⁺: m/z=1015.5; Found: 1015.7.

### Example 43. Methyl((1R,3R)-3-(7-(4-((4-((4-(2-(2,6-dioxopiperidin-3-yl)-1-oxoisoindolin-5-yl)piperazin-1-yl)methyl)piperidin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

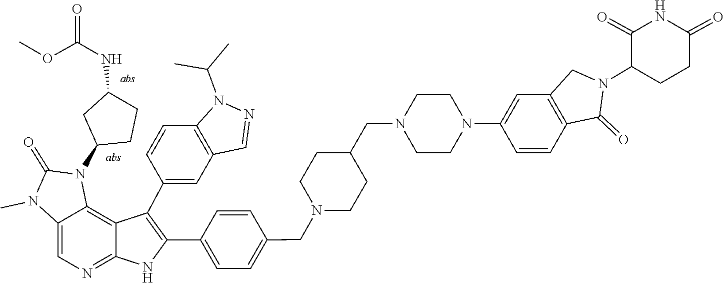 [SMILES: COC(=O)N[C@@H]1CC[C@@H](n2c(=O)n(C)c3cnc4[nH]c(-c5ccc(CN6CCC(CN7CCN(c8ccc9c(c8)CN(C8CCC(=O)NC8=O)C9=O)CC7)CC6)cc5)c(-c5ccc6c(cnn6C(C)C)c5)c4c32)C1]

A procedure analogous to the one described in Example 42 steps 1-3 was used, with the exception that 3-(1-oxo-5-(4-(piperidin-4-ylmethyl)piperazin-1-yl)isoindolin-2-yl)piperidine-2,6-dione (9.6 mg, 0.023 mmol, Tocris Bioscience 7473) was used as the amine in step 2, to prepare the title compound. LCMS for C₅₆H₆₅N₁₂O₆(M+H)⁺: m/z=1001.5; Found: 1001.7.

### Example 44. Methyl((1R,3R)-3-(7-(4-((4-((4-(2-(2,6-dioxopiperidin-3-yl)-1,3-dioxoisoindolin-4-yl)piperazin-1-yl)methyl)piperidin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

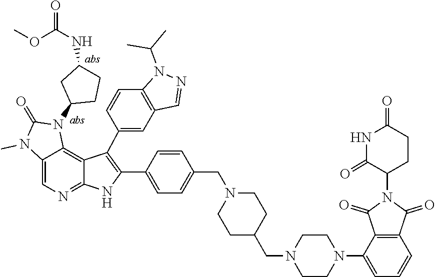 [SMILES: COC(=O)N[C@@H]1CC[C@@H](n2c(=O)n(C)c3cnc4[nH]c(-c5ccc(CN6CCC(CN7CCN(c8cccc9c8C(=O)N(C8CCC(=O)NC8=O)C9=O)CC7)CC6)cc5)c(-c5ccc6c(cnn6C(C)C)c5)c4c32)C1]

A procedure analogous to the one described in Example 42 steps 1-3 was used with the exception that 2-(2,6-dioxopiperidin-3-yl)-4-(4-(piperidin-4-ylmethyl)piperazin-1-yl)isoindoline-1,3-dione (Sigma-Aldrich 923877) was used in step 2 as the amine to prepare the title compound. LCMS for C₅₆H₆₃N₁₂O₇(M+H)⁺: m/z=1015.5; Found: 1015.6.

### Example 45. Methyl((1R,3R)-3-(7-(4-((2-(2-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

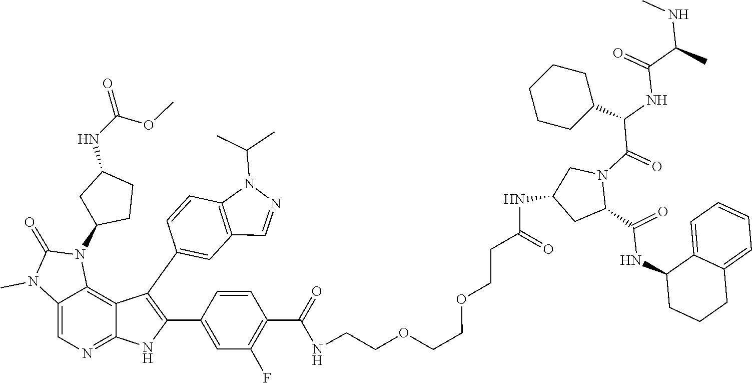 [SMILES: CN[C@@H](C)C(=O)N[C@H](C(=O)N1C[C@@H](NC(=O)CCOCCOCCNC(=O)c2ccc(-c3[nH]c4ncc5c(c4c3-c3ccc4c(cnn4C(C)C)c3)n([C@@H]3CC[C@@H](NC(=O)OC)C3)c(=O)n5C)cc2F)C[C@H]1C(=O)N[C@@H]1CCCc2ccccc21)C1CCCCC1]

### Step 1. tert-Butyl ((S)-1-(((S)-2-((2S,4S)-4-(1-(9H-fluoren-9-yl)-3-oxo-2,7,10-trioxa-4-azatridecan-13-amido)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate

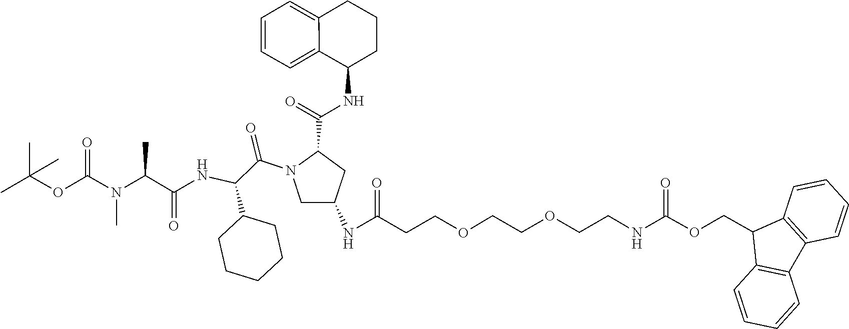 [SMILES: C[C@@H](C(=O)N[C@H](C(=O)N1C[C@@H](NC(=O)CCOCCOCCNC(=O)OCC2c3ccccc3-c3ccccc32)C[C@H]1C(=O)N[C@@H]1CCCc2ccccc21)C1CCCCC1)N(C)C(=O)OC(C)(C)C]

To a solution of 1-(9H-fluoren-9-yl)-3-oxo-2,7,10-trioxa-4-azatridecan-13-oic acid (13.7 mg, 0.034 mmol, Synthonix Corporation SY3H₆E4146F₃), tert-butyl ((S)-1-(((S)-2-((2S,4S)-4-amino-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (20 mg, 0.034 mmol, Tocris Bioscience A 410099.1 amine, cat. #6471), and HATU (19.5 mg, 0.051 mmol) in DCE (0.5 ml) was added DIEA (0.018 ml, 0.10 mmol) and the solution was stirred for 2-3 hours until complete. The crude reaction mixture was injected onto a 4 g silica gel column and purified by CombiFlash chromatography (4 g connected to a 12 g silica gel column, eluting with 0-20% MeOH/DCM) to afford the desired product (28 mg, 85% yield). LCMS for C₅₄H₇₃N₆O₁₀ (M+H)⁺: m/z=965.5; Found: 965.6.

### Step 2. tert-Butyl ((S)-1-(((S)-2-((2S,4S)-4-(3-(2-(2-aminoethoxy)ethoxy)propanamido)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate

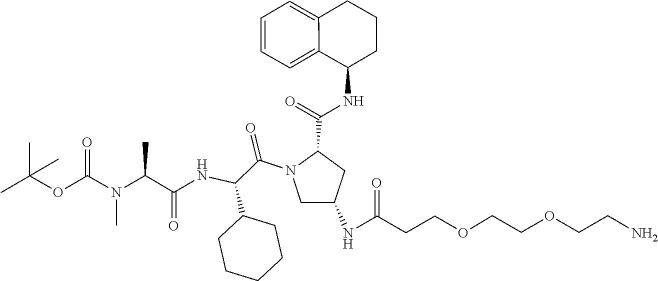 [SMILES: C[C@@H](C(=O)N[C@H](C(=O)N1C[C@@H](NC(=O)CCOCCOCCN)C[C@H]1C(=O)N[C@@H]1CCCc2ccccc21)C1CCCCC1)N(C)C(=O)OC(C)(C)C]

tert-Butyl ((S)-1-(((S)-2-((2S,4S)-4-(1-(9H-fluoren-9-yl)-3-oxo-2,7,10-trioxa-4-azatridecan-13-amido)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (33 mg, 0.034 mmol) was dissolved in dimethylamine (2.0 in THF) (1.0 ml, 2.0 mmol) and stirred at ambient temperature for several hours until complete. The volatiles were removed in-vacuo and the residue was placed under high vacuum overnight and used in the subsequent reaction without further purification. LCMS for C₃₉H₆₃N₆O₈ (M+H)⁺: m/z=743.5; Found: 743.5.

### Step 3. tert-Butyl ((S)-1-(((S)-1-cyclohexyl-2-((2S,4S)-4-(3-(2-(2-(2-fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzamido)ethoxy)ethoxy)propanamido)-2-(((R)-1,2,3,4-tetrahydronaphthalen-1-yl)carbamoyl)pyrrolidin-1-yl)-2-oxoethyl)amino)-1-oxopropan-2-yl)(methyl)carbamate

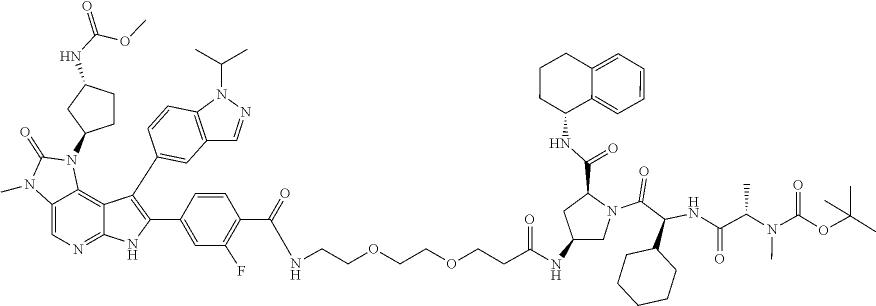 [SMILES: COC(=O)N[C@@H]1CC[C@@H](n2c(=O)n(C)c3cnc4[nH]c(-c5ccc(C(=O)NCCOCCOCCC(=O)N[C@H]6C[C@@H](C(=O)N[C@@H]7CCCc8ccccc87)N(C(=O)[C@@H](NC(=O)[C@H](C)N(C)C(=O)OC(C)(C)C)C7CCCCC7)C6)c(F)c5)c(-c5ccc6c(cnn6C(C)C)c5)c4c32)C1]

A procedure analogous to that described in Example 1 was used with the exception that tert-butyl ((S)-1-(((S)-2-((2S,4S)-4-(3-(2-(2-aminoethoxy)ethoxy)propanamido)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate was used in the amide coupling (step 11 of Example 1). LCMS for C₇₂H₉₄FN₁₃O₁₂ (M+2H)²⁺: m/z=676.3; Found: 676.2.

### Step 4. Methyl((1R,3R)-3-(7-(4-((2-(2-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

To a solution of tert-butyl ((S)-1-(((S)-1-cyclohexyl-2-((2S,4S)-4-(3-(2-(2-(2-fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzamido)ethoxy)ethoxy)propanamido)-2-(((R)-1,2,3,4-tetrahydronaphthalen-1-yl)carbamoyl)pyrrolidin-1-yl)-2-oxoethyl)amino)-1-oxopropan-2-yl)(methyl)carbamate (7.5 mg, 5.6 μmol) in DCM (1 ml) was added TFA (200 μl, 2.6 mmol) and the solution was stirred at ambient temperature. After 1 h, the reaction mixture was diluted with acetonitrile, acidified by the addition of TFA, and purified via preparative HPLC (XBridge C₁₈ column, eluting with a gradient of acetonitrile/water containing 0.1% TFA, at flow rate of 60 mL/min) to afford the desired product. LCMS for C₆₇H₈₆FN₁₃O₁₀ (M+2H)²⁺: m/z=626.3; Found: 626.1.

### Example 46. Methyl((1R,3R)-3-(7-(4-(4-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

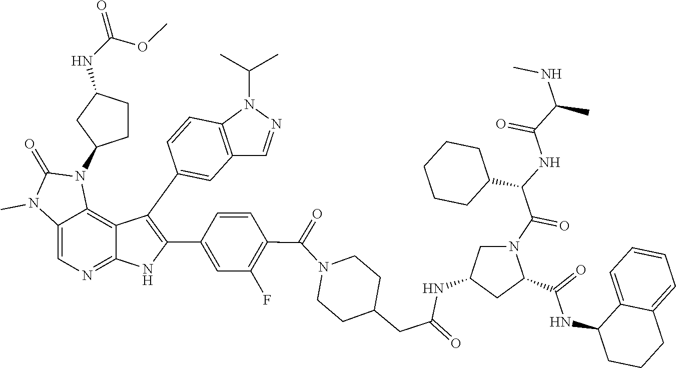 [SMILES: CN[C@@H](C)C(=O)N[C@H](C(=O)N1C[C@@H](NC(=O)CC2CCN(C(=O)c3ccc(-c4[nH]c5ncc6c(c5c4-c4ccc5c(cnn5C(C)C)c4)n([C@@H]4CC[C@@H](NC(=O)OC)C4)c(=O)n6C)cc3F)CC2)C[C@H]1C(=O)N[C@@H]1CCCc2ccccc21)C1CCCCC1]

A procedure analogous to that described in Example 45 was used with the exception that tert-butyl ((S)-1-(((S)-1-cyclohexyl-2-((2S,4S)-4-(2-(1-(2-fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzoyl)piperidin-4-yl)acetamido)-2-(((R)-1,2,3,4-tetrahydronaphthalen-1-yl)carbamoyl)pyrrolidin-1-yl)-2-oxoethyl)amino)-1-oxopropan-2-yl)(methyl)carbamate was used as the amine in the amide coupling step 3. LCMS for C₆₇H₈₃FN₁₃O₈(M+H)⁺: m/z=1216.6; Found: 1216.4.

### Example 47. Methyl((1R,3R)-3-(7-(4-((((trans)-4-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)carbamoyl)cyclohexyl)methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

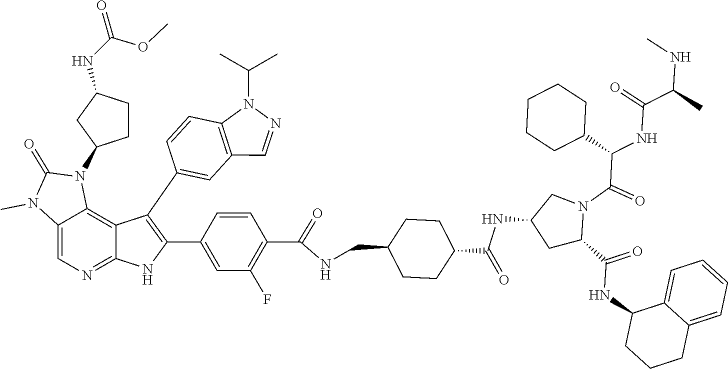 [SMILES: CN[C@@H](C)C(=O)N[C@H](C(=O)N1C[C@@H](NC(=O)[C@H]2CC[C@H](CNC(=O)c3ccc(-c4[nH]c5ncc6c(c5c4-c4ccc5c(cnn5C(C)C)c4)n([C@@H]4CC[C@@H](NC(=O)OC)C4)c(=O)n6C)cc3F)CC2)C[C@H]1C(=O)N[C@@H]1CCCc2ccccc21)C1CCCCC1]

A procedure analogous to that described in Example 45 was used with the exception that tert-butyl ((S)-1-(((S)-1-cyclohexyl-2-((2S,4S)-4-((trans)-4-((2-fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzamido)methyl)cyclohexane-1-carboxamido)-2-(((R)-1,2,3,4-tetrahydronaphthalen-1-yl)carbamoyl)pyrrolidin-1-yl)-2-oxoethyl)amino)-1-oxopropan-2-yl)(methyl)carbamate was used as the amine in the amide coupling step. LCMS for C₆₈H₈₅FN₁₃O₈(M+H)⁺: m/z=1230.6; Found: 1230.4.

### Example 48. Methyl((1R,3R)-3-(7-(4-((((trans-)-4-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)carbamoyl)cyclohexyl)methyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

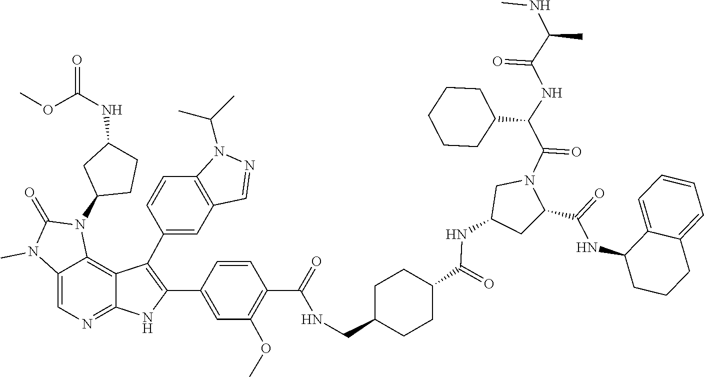 [SMILES: CN[C@@H](C)C(=O)N[C@H](C(=O)N1C[C@@H](NC(=O)[C@H]2CC[C@H](CNC(=O)c3ccc(-c4[nH]c5ncc6c(c5c4-c4ccc5c(cnn5C(C)C)c4)n([C@@H]4CC[C@@H](NC(=O)OC)C4)c(=O)n6C)cc3OC)CC2)C[C@H]1C(=O)N[C@@H]1CCCc2ccccc21)C1CCCCC1]

A procedure analogous to that described in Example 45 was used with the exception that tert-butyl ((S)-1-(((S)-1-cyclohexyl-2-((2S,4S)-4-((trans-)-4-((4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)-2-methoxybenzamido)methyl)cyclohexane-1-carboxamido)-2-(((R)-1,2,3,4-tetrahydronaphthalen-1-yl)carbamoyl)pyrrolidin-1-yl)-2-oxoethyl)amino)-1-oxopropan-2-yl)(methyl)carbamate was used as the amine in the amide coupling step. LCMS for C₆₉H₈₈N₁₃O₉(M+H)⁺: m/z=1242.7; Found: 1242.7.

### Example 49. Methyl((1R,3R)-3-(7-(4-((2-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

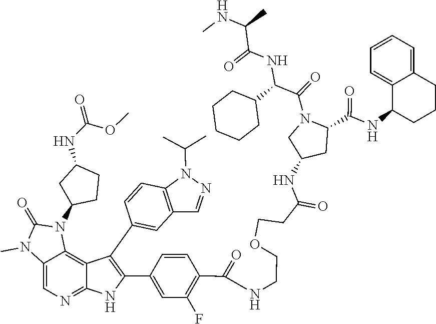 [SMILES: CN[C@@H](C)C(=O)N[C@H](C(=O)N1C[C@@H](NC(=O)CCOCCNC(=O)c2ccc(-c3[nH]c4ncc5c(c4c3-c3ccc4c(cnn4C(C)C)c3)n([C@@H]3CC[C@@H](NC(=O)OC)C3)c(=O)n5C)cc2F)C[C@H]1C(=O)N[C@@H]1CCCc2ccccc21)C1CCCCC1]

A procedure analogous to that described in Example 45 was used with the exception that tert-butyl ((S)-1-(((S)-2-((2S,4S)-4-(3-(2-aminoethoxy)propanamido)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate was used as the amine in the amide coupling step. LCMS for C₆₅H₈₁FN₁₃O₉(M+H)⁺: m/z=1206.6; Found: 1206.4.

### Example 50. Methyl((1R,3R)-3-(7-(4-((2-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

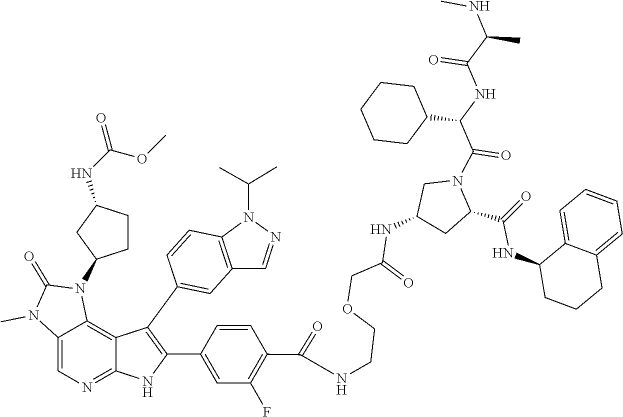 [SMILES: CN[C@@H](C)C(=O)N[C@H](C(=O)N1C[C@@H](NC(=O)COCCNC(=O)c2ccc(-c3[nH]c4ncc5c(c4c3-c3ccc4c(cnn4C(C)C)c3)n([C@@H]3CC[C@@H](NC(=O)OC)C3)c(=O)n5C)cc2F)C[C@H]1C(=O)N[C@@H]1CCCc2ccccc21)C1CCCCC1]

A procedure analogous to that described in Example 45 was used with the exception that tert-butyl ((S)-1-(((S)-2-((2S,4S)-4-(2-(2-aminoethoxy)acetamido)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate was used as the amine in the amide coupling step. LCMS for C₆₄H₇₉FN₁₃O₉(M+H)⁺: m/z=1192.6; Found: 1192.4.

### Example 51. Methyl((1R,3R)-3-(7-(4-((2-(2-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

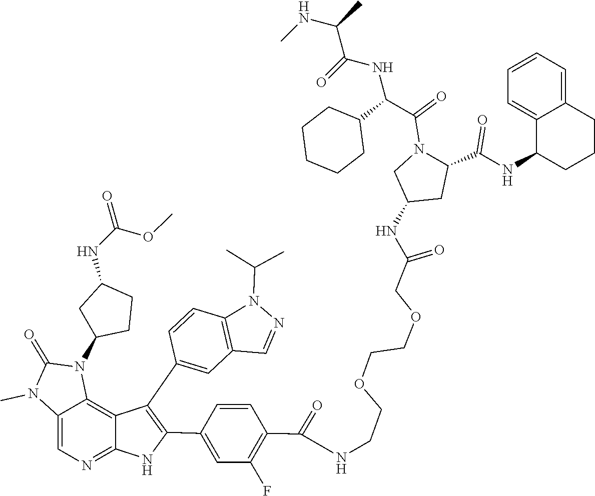 [SMILES: CN[C@@H](C)C(=O)N[C@H](C(=O)N1C[C@@H](NC(=O)COCCOCCNC(=O)c2ccc(-c3[nH]c4ncc5c(c4c3-c3ccc4c(cnn4C(C)C)c3)n([C@@H]3CC[C@@H](NC(=O)OC)C3)c(=O)n5C)cc2F)C[C@H]1C(=O)N[C@@H]1CCCc2ccccc21)C1CCCCC1]

A procedure analogous to that described in Example 45 was used with the exception that tert-butyl ((S)-1-(((S)-2-((2S,4S)-4-(2-(2-(2-aminoethoxy)ethoxy)acetamido)-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate was used as the amine in the amide coupling step. LCMS for C₆₆H₈₃FN₁₃O₁₀ (M+H)⁺: m/z=1236.6; Found: 1236.4.

### Example 52. Methyl((1R,3R)-3-(7-(4-((2-(2-(3-((2-(2,6-dioxopiperidin-3-yl)-1-oxoisoindolin-5-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

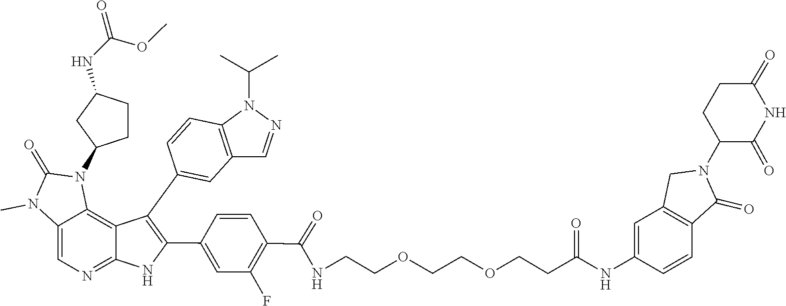 [SMILES: COC(=O)N[C@@H]1CC[C@@H](n2c(=O)n(C)c3cnc4[nH]c(-c5ccc(C(=O)NCCOCCOCCC(=O)Nc6ccc7c(c6)CN(C6CCC(=O)NC6=O)C7=O)c(F)c5)c(-c5ccc6c(cnn6C(C)C)c5)c4c32)C1]

### Step 1. tert-Butyl (2-(2-(3-((2-(2,6-dioxopiperidin-3-yl)-1-oxoisoindolin-5-yl)amino)-3-oxopropoxy)ethoxy)ethyl)carbamate

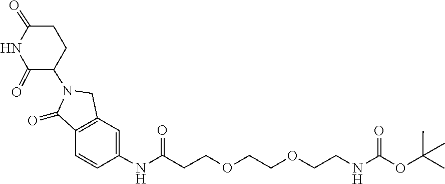 [SMILES: CC(C)(C)OC(=O)NCCOCCOCCC(=O)Nc1ccc2c(c1)CN(C1CCC(=O)NC1=O)C2=O]

To a mixture of 2,2-dimethyl-4-oxo-3,8,11-trioxa-5-azatetradecan-14-oic acid (22 mg, 0.079 mmol, Apollo Scientific BIPG 1800) in DMF (0.7 ml) was added sequentially HATU (45 mg, 0.12 mmol), DIEA (0.035 ml, 0.20 mmol), and 3-(5-amino-1-oxoisoindolin-2-yl)piperidine-2,6-dione (20 mg, 0.079 mmol, Sigma-Aldrich 911690). Upon completion, the crude reaction mixture was purified by CombiFlash chromatography (12 g silica gel column, eluting with 0-15% methanol/dichloromethane) to afford the desired product (17 mg, 41% yield). LCMS for C₂₀H₂₇N₄O₆ (M−Boc+H)⁺: m/z=419.2; Found: 419.2.

### Step 2. 3-(2-(2-Aminoethoxy)ethoxy)-N-(2-(2,6-dioxopiperidin-3-yl)-1-oxoisoindolin-5-yl)propanamide

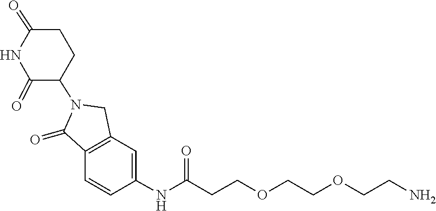 [SMILES: NCCOCCOCCC(=O)Nc1ccc2c(c1)CN(C1CCC(=O)NC1=O)C2=O]

To a solution of tert-butyl (2-(2-(3-((2-(2,6-dioxopiperidin-3-yl)-1-oxoisoindolin-5-yl)amino)-3-oxopropoxy)ethoxy)ethyl)carbamate in DCM (1 ml) was added TFA (200 μl, 2.6 mmol) and the solution was stirred at ambient temperature. The volatiles were removed in-vacuo and the residue was azeotropically washed with acetonitrile several times and used directly in the subsequent step without further purification. LCMS for C₂₀H₂₇N₄O₆ (M+H)⁺: m/z=419.2; Found: 419.2.

### Step 3. Methyl((1R,3R)-3-(7-(4-((2-(2-(3-((2-(2,6-dioxopiperidin-3-yl)-1-oxoisoindolin-5-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

A procedure analogous to that described in Example 1 was used with the exception that 3-(2-(2-aminoethoxy)ethoxy)-N-(2-(2,6-dioxopiperidin-3-yl)-1-oxoisoindolin-5-yl)propanamide was used as the amine in the amide coupling step 11. LCMS for C₅₃H₅₇FN₁₁O₁₀ (M+H)⁺: m/z=1026.4; Found: 1026.4.

### Example 53. Methyl((1R,3R)-3-(7-(4-(4-(2-((2-(2,6-dioxopiperidin-3-yl)-1-oxoisoindolin-5-yl)amino)-2-oxoethyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

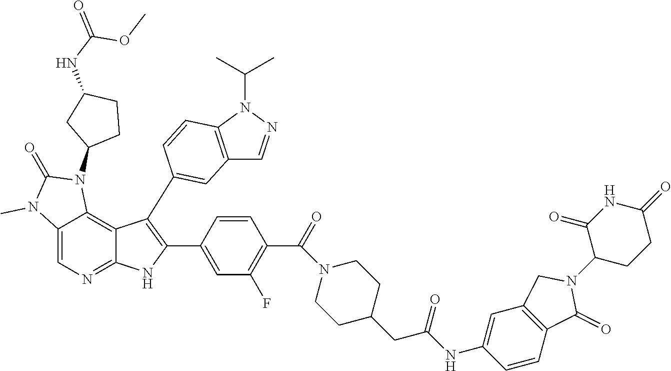 [SMILES: COC(=O)N[C@@H]1CC[C@@H](n2c(=O)n(C)c3cnc4[nH]c(-c5ccc(C(=O)N6CCC(CC(=O)Nc7ccc8c(c7)CN(C7CCC(=O)NC7=O)C8=O)CC6)c(F)c5)c(-c5ccc6c(cnn6C(C)C)c5)c4c32)C1]

A procedure analogous to that described in Example 52 was used with the exception that 2-(1-(tert-butoxycarbonyl)piperidin-4-yl)acetic acid was used as the carboxylic acid in step 1. LCMS for C₅₃H₅₅FN₁₁O₈(M+H)⁺: m/z=992.4; Found: 992.4.

### Example 54. Methyl((1R,3R)-3-(7-(4-((((trans-)-4-((2-(2,6-dioxopiperidin-3-yl)-1-oxoisoindolin-5-yl)carbamoyl)cyclohexyl)methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

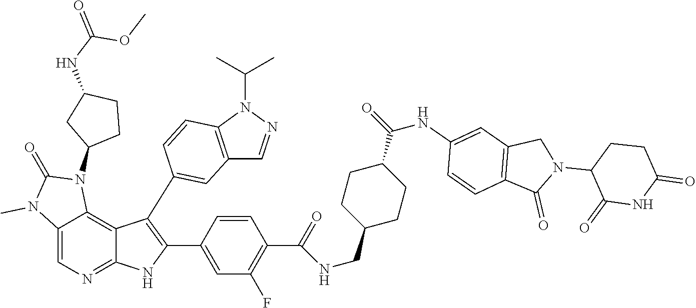 [SMILES: COC(=O)N[C@@H]1CC[C@@H](n2c(=O)n(C)c3cnc4[nH]c(-c5ccc(C(=O)NC[C@H]6CC[C@H](C(=O)Nc7ccc8c(c7)CN(C7CCC(=O)NC7=O)C8=O)CC6)c(F)c5)c(-c5ccc6c(cnn6C(C)C)c5)c4c32)C1]

A procedure analogous to that described in Example 52 was used with the exception that (trans)-4-(((tert-butoxycarbonyl)amino)methyl)cyclohexane-1-carboxylic acid was used as the carboxylic acid in step 1. LCMS for C₅₄H₅₇FN₁₁O₈(M+H)⁺: m/z=1006.4; Found: 1006.5.

### Example 55. Methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

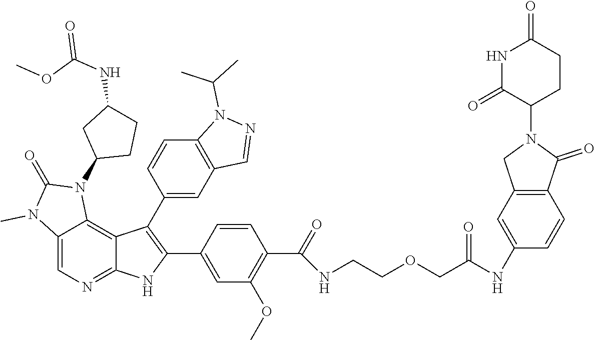 [SMILES: COC(=O)N[C@@H]1CC[C@@H](n2c(=O)n(C)c3cnc4[nH]c(-c5ccc(C(=O)NCCOCC(=O)Nc6ccc7c(c6)CN(C6CCC(=O)NC6=O)C7=O)c(OC)c5)c(-c5ccc6c(cnn6C(C)C)c5)c4c32)C1]

A procedure analogous to that described in Example 52 was used with the exception that 2-(2-((tert-butoxycarbonyl)amino)ethoxy)acetic acid was used as the carboxylic acid in step 1. LCMS for C₅₁H₅₄N₁₁O₁₀ (M+H)⁺: m/z=980.4; Found: 980.6.

### Example 56. Methyl((1R,3R)-3-(7-(4-((((trans)-4-((2-(2,6-dioxopiperidin-3-yl)-1-oxoisoindolin-5-yl)carbamoyl)cyclohexyl)methyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

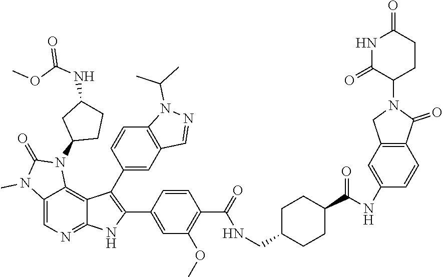 [SMILES: COC(=O)N[C@@H]1CC[C@@H](n2c(=O)n(C)c3cnc4[nH]c(-c5ccc(C(=O)NC[C@H]6CC[C@H](C(=O)Nc7ccc8c(c7)CN(C7CCC(=O)NC7=O)C8=O)CC6)c(OC)c5)c(-c5ccc6c(cnn6C(C)C)c5)c4c32)C1]

A procedure analogous to that described in Example 52 was used with the exception that trans-4-(((tert-butoxycarbonyl)amino)methyl)cyclohexane-1-carboxylic acid was used as the carboxylic acid in step 1. LCMS for C₅₅H₆₀N₁₁O₉(M+H)⁺: m/z=1018.4; Found: 1018.5.

### Example 57. Methyl((1R,3R)-3-(7-(4-(2-((2-(2,6-dioxopiperidin-3-yl)-1-oxoisoindolin-5-yl)carbamoyl)morpholine-4-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

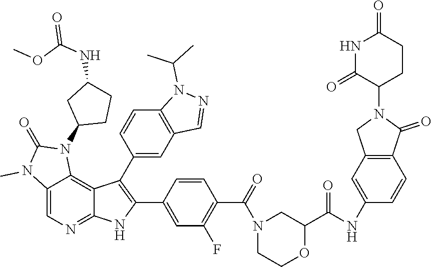 [SMILES: COC(=O)N[C@@H]1CC[C@@H](n2c(=O)n(C)c3cnc4[nH]c(-c5ccc(C(=O)N6CCOC(C(=O)Nc7ccc8c(c7)CN(C7CCC(=O)NC7=O)C8=O)C6)c(F)c5)c(-c5ccc6c(cnn6C(C)C)c5)c4c32)C1]

A procedure analogous to that described in Example 52 was used with the exception that 4-(tert-butoxycarbonyl)morpholine-2-carboxylic acid was used as the carboxylic acid in step 1. LCMS for C₅₁H₅₁FN₁₁O₉(M+H)⁺: m/z=980.4; Found: 980.5.

### Example 58. Methyl((1R,3R)-3-(7-(4-(4-((2-(2,6-dioxopiperidin-3-yl)-1-oxoisoindolin-5-yl)carbamoyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

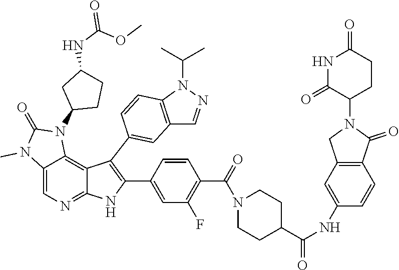 [SMILES: COC(=O)N[C@@H]1CC[C@@H](n2c(=O)n(C)c3cnc4[nH]c(-c5ccc(C(=O)N6CCC(C(=O)Nc7ccc8c(c7)CN(C7CCC(=O)NC7=O)C8=O)CC6)c(F)c5)c(-c5ccc6c(cnn6C(C)C)c5)c4c32)C1]

A procedure analogous to that described in Example 52 was used with the exception that 1-(tert-butoxycarbonyl)piperidine-4-carboxylic acid was used as the carboxylic acid in step 1. LCMS for C₅₂H₅₃FN₁₁O₈(M+H)⁺: m/z=978.4; Found: 978.4.

### Example 59. Methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

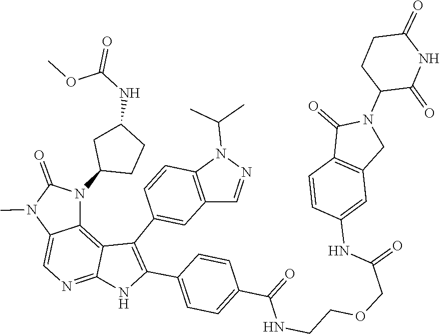 [SMILES: COC(=O)N[C@@H]1CC[C@@H](n2c(=O)n(C)c3cnc4[nH]c(-c5ccc(C(=O)NCCOCC(=O)Nc6ccc7c(c6)CN(C6CCC(=O)NC6=O)C7=O)cc5)c(-c5ccc6c(cnn6C(C)C)c5)c4c32)C1]

A procedure analogous to that described in Example 52 was used with the exception that 2-(2-((tert-butoxycarbonyl)amino)ethoxy)acetic acid was used as the carboxylic acid in step 1. LCMS for C₅₀H₅₂N₁₁O₉(M+H)⁺: m/z=950.4; Found: 950.4.

### Example 60. Methyl((1R,3R)-3-(7-(4-(4-((2-(2,6-dioxopiperidin-3-yl)-1-oxoisoindolin-5-yl)carbamoyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

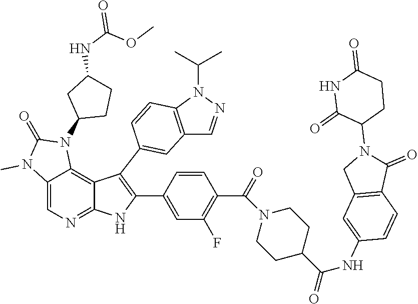 [SMILES: COC(=O)N[C@@H]1CC[C@@H](n2c(=O)n(C)c3cnc4[nH]c(-c5ccc(C(=O)N6CCC(C(=O)Nc7ccc8c(c7)CN(C7CCC(=O)NC7=O)C8=O)CC6)c(F)c5)c(-c5ccc6c(cnn6C(C)C)c5)c4c32)C1]

A procedure analogous to that described in Example 52 was used with the exception that 1-(tert-butoxycarbonyl)piperidine-4-carboxylic acid was used as the carboxylic acid in step 1. LCMS for C₅₂H₅₄N₁₁O₈(M+H)⁺: m/z=960.4; Found: 960.6.

### Example 61. Methyl((1R,3R)-3-(7-(4-(4-(2-((2-(2,6-dioxopiperidin-3-yl)-1-oxoisoindolin-5-yl)amino)-2-oxoethyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

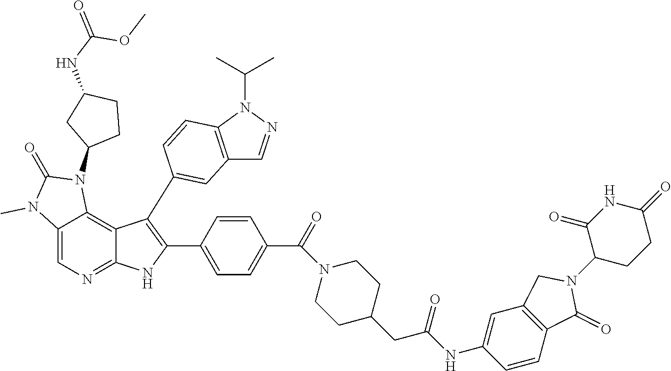 [SMILES: COC(=O)N[C@@H]1CC[C@@H](n2c(=O)n(C)c3cnc4[nH]c(-c5ccc(C(=O)N6CCC(CC(=O)Nc7ccc8c(c7)CN(C7CCC(=O)NC7=O)C8=O)CC6)cc5)c(-c5ccc6c(cnn6C(C)C)c5)c4c32)C1]

A procedure analogous to that described in Example 52 was used with the exception that 2-(1-(tert-butoxycarbonyl)piperidin-4-yl)acetic acid was used as the carboxylic acid in step 1. LCMS for C₅₃H₅₆N₁₁O₈(M+H)⁺: m/z=974.4; Found: 974.5.

### Example 62. Methyl((1R,3R)-3-(7-(4-(4-(2-(2,6-dioxopiperidin-3-yl)-1,3-dioxoisoindolin-5-yl)piperazine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

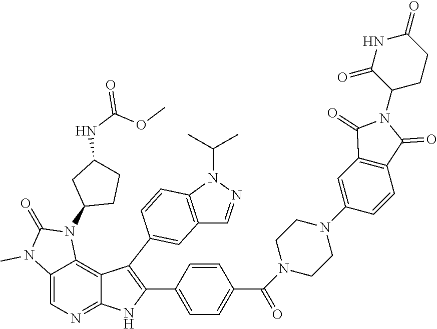 [SMILES: COC(=O)N[C@@H]1CC[C@@H](n2c(=O)n(C)c3cnc4[nH]c(-c5ccc(C(=O)N6CCN(c7ccc8c(c7)C(=O)N(C7CCC(=O)NC7=O)C8=O)CC6)cc5)c(-c5ccc6c(cnn6C(C)C)c5)c4c32)C1]

A procedure analogous to that described in Example 52 was used with the exception that tert-butyl 4-(2-(2,6-dioxopiperidin-3-yl)-1,3-dioxoisoindolin-5-yl)piperazine-1-carboxylate (Angene AG01X0W1 [2222114-64-7] was used as the starting material in step 2. LCMS for C₅₀H₅₀N₁₁O₈(M+H)⁺: m/z=932.4; Found: 932.5.

### Example 63. Methyl((1R,3R)-3-(7-(4-(4-((4-(2-(2,6-dioxopiperidin-3-yl)-3-oxoisoindolin-4-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

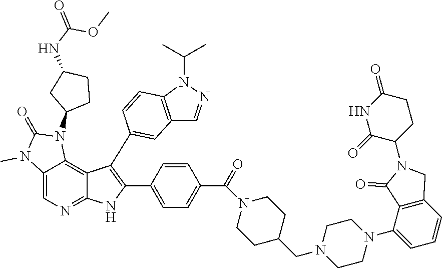 [SMILES: COC(=O)N[C@@H]1CC[C@@H](n2c(=O)n(C)c3cnc4[nH]c(-c5ccc(C(=O)N6CCC(CN7CCN(c8cccc9c8C(=O)N(C8CCC(=O)NC8=O)C9)CC7)CC6)cc5)c(-c5ccc6c(cnn6C(C)C)c5)c4c32)C1]

### Step 1. tert-butyl 4-((4-(2-(2,6-dioxopiperidin-3-yl)-3-oxoisoindolin-4-yl)piperazin-1-yl)methyl)piperidine-1-carboxylate

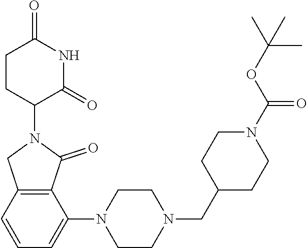 [SMILES: CC(C)(C)OC(=O)N1CCC(CN2CCN(c3cccc4c3C(=O)N(C3CCC(=O)NC3=O)C4)CC2)CC1]

A solution of 3-(7-bromo-1-oxoisoindolin-2-yl)piperidine-2,6-dione (40 mg, 0.124 mmol), tert-butyl 4-(piperazin-1-ylmethyl)piperidine-1-carboxylate (70 mg, 0.25 mmol), Pd-PEPPSI™-IPent catalyst (4.9 mg, 6.2 μmol, [1158652-41-5]), and cesium carbonate (121 mg, 0.371 mmol) in 1,4-dioxane (1.2 mL) (0.1 M) was de-gassed and purged with N₂ (g) several times prior to heating at 100° C. in a sealed vial. Upon completion, the crude reaction mixture was purified by CombiFlash chromatography (12 g silica gel column, eluting with 0-15% methanol (spiked with 7% ammonia)/dichloromethane) to afford the desired product. LCMS for C₂₈H₄₀N₅O₅ (M+H)⁺: m/z=526.3; Found: 526.3.

### Step 2. 3-(1-Oxo-7-(4-(piperidin-4-ylmethyl)piperazin-1-yl)isoindolin-2-yl)piperidine-2,6-dione

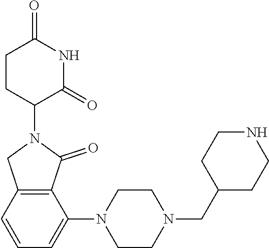 [SMILES: O=C1CCC(N2Cc3cccc(N4CCN(CC5CCNCC5)CC4)c3C2=O)C(=O)N1]

A procedure analogous to that described in Example 52 step 2 was used starting with tert-butyl 4-((4-(2-(2,6-dioxopiperidin-3-yl)-3-oxoisoindolin-4-yl)piperazin-1-yl)methyl)piperidine-1-carboxylate. LCMS for C₂₃H₃₂N₅O₃ (M+H)⁺: m/z=426.2; Found: 426.3.

### Step 3. Methyl((1R,3R)-3-(7-(4-(4-((4-(2-(2,6-dioxopiperidin-3-yl)-3-oxoisoindolin-4-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

A procedure analogous to that described in Example 52 step 3 was used starting with 3-(1-oxo-7-(4-(piperidin-4-ylmethyl)piperazin-1-yl)isoindolin-2-yl)piperidine-2,6-dione, TFA. LCMS for C₅₆H₆₃N₁₂O₇(M+H)⁺: m/z=1015.5; Found: 1015.7.

### Example A. JAK2 LanthaScreen JH1 Binding Assay

JAK2 JH1 binding assay utilizes catalytic domain (JH1, amino acids 826-1132) of human JAK2 expressed as N-terminal FLAG-tagged, biotinylated protein in a baculovirus expression system (Carna Biosciences, Product #08-445-20N). The assay was conducted in black 384-well polystyrene plates in a final reaction volume of 20 μL. JAK2 JH1 (1.5 nM) was incubated with compounds (100 nL serially diluted in DMSO) in the presence of 50 nM fluorescent JAK2-JH1 tracer and 0.5 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 2 hours at 25° C., LanthaScreen signals were read on a PHERAstar FS plate reader (BMG LABTECH). Data was analyzed with IDBS XLfit and GraphPad Prism 5.0 software using a four parameter dose response curve to determine IC₅₀ for each compound.

### Example B. JAK2 LanthaScreen JH2-V617F Binding Assay

JAK2 JH2-V617F binding assay utilizes pseudo-kinase domain (JH2, amino-acids 536-812 with 3 surface mutations W659A, W777A, F₇₉₄H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC₅₀ for each compound.

### Example C. STAT5 (Tyr694) Phosphorylation Cell Based Assay

SET-2 cells was purchased from DSMZ (Germany). RPMI1640 medium, Fetal Bovine Serum and 384-white sold flat-bottom small volume plate were purchased from Thermo Fisher Scientific (Waltham, MA). Phospho-STAT5 (Tyr694) HTRF kit was purchased from Perkin Elmer (Waltham, MA).

SET-2 cells were cultured in RPMI media with 20% FBS at 37° C. in humidified incubator supplied with 5% CO₂. On the day of assay, the cells were centrifuged to remove the culture media and resuspended with prewarmed RPMI with 10% FBS. Testing compounds were prepared by serial dilution in DMSO and 50 nL/well test compounds were transferred to the 384 white low volume cell culture plate (Greiner Bio-one) by ECHO liquid handler (Labcyte). The cells were then dispensed with Multidrop (Thermo Fisher, Waltham, MA) at 10 μL/well (7×10⁶ cells/mL) with 0.5% DMSO in the final assay. After the treated cells were incubated for 2 hours at 37° C./5% CO₂ incubator, 4 μL/well supplemented lysis buffer (100× blocking buffer diluted 25 fold in 4× lysis buffer, Perkin Elmer) were added and incubated at room temperature for 90 min on orbital shaker at 600 rpm. Phospho-STAT5 Cryptate antibody and Phospho-STAT5 d2 antibody (1:1 vol/vol) were premixed and diluted 20 fold with in the detection buffer (Perkin Elmer). 4 μL of the premixed antibody solution were added to each well followed with 24 hours incubation at room temperature on orbital shaker at 600 rpm. The product activity was determined by measuring the fluorescence at 620 nm and 665 nm on Pherastar microplate reader (BMG Labtech). A ratio is calculated (665/620) for each well. Wells with DMSO only served as the positive controls and wells containing high concentration of control compound were used as negative controls. IC₅₀ determination was performed by fitting the curve of percent control activity versus the log of the compound concentration using the GraphPad Prism 7.0 software.

### Example D. pSTAT5 SET-2 Whole Blood MSD Assay

The following were used in this example: SET-2 cells shipped from ABS, RPMI1640 medium, Fetal Bovine Serum from Gibco, Phospho-STAT5a,b Whole Cell Lysate Kit from Mesoscale, Retronectin (Recombinant Human Fibronectin Fragment) from TaKaRa, 96 well cell culture plate flat bottom from Corning, lysis buffer from Cell Signaling Technology, sterile PBS from Gibco, and Whole Blood from by BioIVT.

SET-2 cells were cultured in RPMI media with 20% FBS at 37° C. humidified incubator supplied with 5% CO₂. On the day of the assay, 40 uL per well of diluted retronectin working solution was added to sterile 96-well clear flat bottom tissue culture plates (1:200 dilution of retronectin in sterile PBS) and incubated at 37° C. for 1 hour. After 1 hour, the retronectin working solution was aspirated and SET-2 cells were seeded at 100,000/well in RPMI+20% FBS. Cells were incubated overnight at 37° C. and 5% CO₂. The next day, the medium was removed and 40 μL of diluted compounds in serum-free RPMI and whole blood was added to the cells for a 2 hour incubation at 37° C. and 5% CO₂. The compound starting concentration is 20 μM and 2.5 fold serial diluted down to 0.21 nM. The whole blood and compound mixture was then aspirated off using the BlueCat plate washer and washed 1× with PBS. Cells were lysed by adding 40 μL of 3× complete lysis buffer and shaken at room temperature for 45-60 minutes (complete lysis buffer consists of Cell Signaling Technology lysis buffer diluted to 3× and supplemented with Mesoscale's protease inhibitor, phosphatase I inhibitor, and phosphatase II inhibitor). The pSTAT5 MSD plates were blocked by adding 150 μL of Blocker A solution per well to the MSD plates and incubated at room temperature with shaking at 400 rpm for one hour or longer. After blocking, plates were washed 3× with 300 μL/well of 1× Tris Wash Buffer. After lysis, added 25 μL per well of lysed sample was added to MSD plate and incubated with shaking at 4° C. wrapped in foil overnight. The next day, plates were washed again and 25 μL/well of diluted 1× Detection Antibody Solution, wrapped in foil, and incubated at room temperature with shaking at 400 rpm for 1 hour. Plates were then washed again and lastly, 150 μL/well of 1× Read Buffer T was added to all wells. Plates were analyzed on MSD Discovery within 5 minutes. Inhibition of pSTAT5 signaling was calculated against a control as inhibitory concentration for 50% inhibition (IC₅₀). Data analysis was performed in GraphPad Prism using a 4-parameter fit and data was reported as Average±SD.

### Example E. Western Blot Analysis of JAK2 Dependent SET2 Cells Treated with Compounds of the Present Disclosure

SET2 cells (DSMZ, catalog number ACC 608) are isolated from a 71-year old woman with human essential thrombocythemia and carry the JAK2 V617F mutation.

SET2 cells were cultured in RPMI culture medium (Gibco, catalog number 11875085) supplemented with 1% penicillin-streptomycin-glutamine (Thermo Fisher Scientific, catalog number 10378016) and 20% fetal bovine serum (Gibco, catalog number 10438034). Cultured cells were maintained at 37° C., 5% CO₂ in the incubator.

Approximately 1,000,000 cells/well were added in 6-well plate containing 1.5 ml culture media with specified concentrations of compounds of the present disclosure or DMSO control. Cells were treated with concentrations of 10 uM, 5 uM, 1 uM, and 0.5 uM. Proteasome inhibitor MG-132 (Cell signaling, catalog number 2194) was added at 20 uM to an additional 10 uM compound treatment. The treatment period was 8 hours. After 8 hours, cells were collected in 1.5 mL microcentrifuge tubes. Tubes were centrifuged at 1500 rpm for 5 min. Supernatant was aspirated and samples were washed with 1 mL of cold PBS. The samples were centrifuged again at 1500 rpm for 5 min, supernatant was aspirated. The sample pellets were lysed using 150 μL of 1× lysing buffer (Cell Signaling Technologies—catalog number 9803S) supplemented with 1× Halt™ Protease and Phosphatase Inhibitor Cocktail (Thermo Fisher Scientific—catalog number 78447). Using Pierce™ BCA Protein Assay Kit (Thermo Fisher Scientific—catalog number 23225), protein concentration of the samples were quantitated. After normalizing sample concentration to approximately 20 ug/well, samples were loaded onto 4-12% Tris-glycine gels. Gels were transferred to nitrocellulose membranes using iBlot 2 Dry Blotting System and iBlot™ 2 Transfer Stacks, nitrocellulose (Thermo Fisher Scientific—catalog number IB23001). Following transfer, membranes were blocked with StartingBlock (Thermo Fisher Scientific—catalog number 37543). After 1 hour of blocking membranes, they were probed for JAK2, pSTAT5, STAT5, CRBN, and GAPDH (Cell Signaling—catalog numbers 3230, 4322, 25656, 71810, and 5174 respectively).

Compounds of the present disclosure were screened at various concentrations to determine their effect on JAK2 protein degradation in SET2 cells, using methods as described above. DMSO was used as the vehicle control with no PROTAC® added. Co-administration of 20 μM of the proteasome inhibitor MG-132 with the PROTAC® compound at its highest concentration (i.e., 5 or 10 μM) tested was conducted to support the hypothesis that the degradation was driven by the ubiquitin-proteosome system (UPS). Degradation was evaluated by western blot densitometry quantitation using the FluorChem M instrument software to analyze the intensity of the JAK2 band signal at various concentrations of PROTAC® compound vs. the JAK2 band signal of the DMSO vehicle. Table 3 summarizes the JAK2 degradation data for selected PROTAC® compounds in SET2 cells. It is noted that ˜82% of JAK2 exists as the V617F mutant, with the remaining 18% of JAK2 being WT. Therefore, if the V617F mutant JAK2 is selectively degraded over the WT, the maximum degradation is ˜80%. Degradation was graded as A: ≥600% degradation; B: ≥40% to <60% degradation; C: ≥20% to <40% degradation, D: <20% to ≥10%; E<10%. ** refers to degradation at 0.5 μM.

TABLE 3
Western Blot Quantitation of JAK2 Degradation
of Selected Compounds in SET2 Cells

| | | JAK2 % Degradation | |
|---|---|---|---|
| | E3 Ligase | in SET2 | |
| Example | target | 5 μM | 10 μM |
| 33 | CRBN | | A |
| 1 | CRBN | | A |
| 35 | CRBN | | A |
| 36 | CRBN | | A |
| 32 | CRBN | B** | |
| 34 | CRBN | A** | |
| 28 | CRBN | | B |
| 51 | IAP | | B |
| 47 | IAP | | C |
| 14 | VHL | | C |
| 61 | CRBN | | C |
| 31 | IAP | | C |
| 24 | CRBN | | C |
| 53 | CRBN | | C |
| 54 | CRBN | | C |
| 60 | CRBN | | C |
| 27 | CRBN | | D |
| 46 | IAP | | D |
| 17 | CRBN | | E |
| 54 | CRBN | | C |
| 58 | CRBN | | E |
| 37 | CRBN | C | |
| 38 | CRBN | A | |
| 40 | CRBN | C | |
| 41 | CRBN | C | |
| 42 | CRBN | A | |
| 43 | CRBN | A | |
| 44 | CRBN | C | |
| 62 | CRBN | E | |
| 63 | CRBN | C | |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 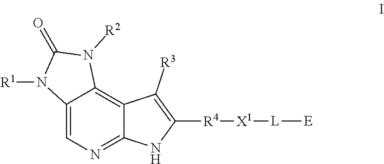
or a pharmaceutically acceptable salt thereof, wherein:
R¹ is selected from H, C₁₋₆ alkyl, C₂₋₆ alkenyl, and C₂₋₆ alkynyl, wherein the C₁₋₆ alkyl, C₂₋₆ alkenyl, and C₂₋₆ alkynyl of R¹ are each optionally substituted with 1, 2, or 3 independently selected R^{1A} substituents;
each R^{1A} is independently selected from halo, oxo, CN, NO₂, OR^{a11}, SR^{a11}, NHOR^{a11}, C(O)R^{b11}, C(O)NR^{c11}R^{d11}, C(O)NR^{c11}(OR^{a11}), C(O)OR^{a11}, OC(O)R^{b11}, OC(O)NR^{c11}R^{d11}, NR^{c11}R^{d11}, NR^{c11}NR^{c11}R^{d11}, NR^{c11}C(O)R^{b11}, NR^{c11}C(O)OR^{a11}, NR^{c11}C(O)NR^{c11}R^{d11}, C(═NR^{e11})R^{b11}, C(═NR^{e11})NR^{c11}R^{d11}, NR^{c11}C(═NR^{e11})NR^{c11}R^{d11}, NR^{c11}C(═NR^{e11})R^{b11}, NR^{c11}S(O)R^{b11}, NR^{c11}S(O)NR^{c11}R^{d11}, NR^{c11}S(O)₂R^{b11}, NR^{c11}S(O)(═NR^{e11})R^{b11}, NR^{c11}S(O)₂NR^{c11}R^{d11}, S(O)R^{b11}, S(O)NR^{c11}R^{d11}, S(O)₂R^{b11}, S(O)₂NR^{c11}R^{d11} OS(O)(═NR^{e11})R^{b11}, and OS(O)₂R^{b11};
each R^{a11}, R^{b11}, R^{c11}, and R^{d11} is independently selected from H, C₁₋₆ alkyl, C₁₋₆ haloalkyl, C₂₋₆ alkenyl, and C₂₋₆ alkynyl, wherein the C₁₋₆ alkyl, C₂₋₆ alkenyl, and C₂₋₆ alkynyl of R^{a11}, R^{b11}, R^{c11} and R^{d11} are each optionally substituted with 1, 2, 3, 4, 5, 6, 7, or 8 independently selected R^{M} substituents;
each R^{e11} is independently selected from H, OH, CN, C₁₋₆ alkyl, C₁₋₆ alkoxy, C₁₋₆ haloalkyl, C₁₋₆ haloalkoxy, C₂₋₆ alkenyl, and C₂₋₆ alkynyl;
R² is selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, 4, 5, 6, 7, or 8 independently selected R^{2A} substituents;
each R^{2A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a21}, SR^{a21}, NHOR^{a21}, C(O)R^{b21}, C(O)NR^{c21}R^{d21}, C(O)NR^{c21}(OR^{a21}), C(O)OR^{a21}, OC(O)R^{b21}, OC(O)NR^{c21}R^{d21} NR^{c21}R^{d21}, NR^{c21}NR^{c21}R^{d21}, NR^{c21}C(O)R^{b21}, NR^{c21}C(O)OR^{a21}, NR^{c21}C(O)NR^{c21}R^{d21}, C(═NR^{e21})R^{b21}, C(═NR^{e21})NR^{c21}R^{d21}, NR^{c21}C(═NR^{e21})R^{c21}R^{d21}, NR^{c21}C(═NR^{e21})R^{b21}, NR^{c21}S(O)R^{b21}, NR^{c21}S(O)NR^{c21}R^{d21}, NR^{c21}S(O)₂R^{b21}, NR^{c21}S(O)(═NR^{e21})R^{b21}, NR^{c21}S(O)₂NR^{c21}R^{d21} S(O)R^{b21}, S(O)NR^{c21}R^{d21}, S(O)₂R^{b21}, S(O)₂NR^{c21}R^{d21} OS(O)(═NR^{e21})R^{b21}, OS(O)₂R^{b21}, SF₅, P(O)R^{f21}R^{g21}, OP(O)(OR^{h21})(ORⁱ²¹), P(O)(OR^{h21})(OR²¹), and BR^{j21}R^{k21}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2A} are each optionally substituted with 1, 2, 3, 4, 5, 6, 7, or 8 independently selected R^{2B} substituents;
each R^{a21}, R^{c21}, and R^{d21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a21}, R^{c21} and R^{d21} are each optionally substituted with 1, 2, 3, 4, 5, 6, 7, or 8 independently selected R^{2B} substituents;
or, any R^{c21} and R^{d21} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2B} substituents;
each R^{b21} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-membered heterocycloalkyl)-C₁₋₆ alkyl-, wherein the C₁₋₆ alkyl, C₂₋₆ alkenyl, C₂₋₆ alkynyl, C₆-10 aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b21} are each optionally substituted with 1, 2, 3, 4, 5, 6, 7, or 8 independently selected R^{2B} substituents;
each R^{e21} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f21} and R^{g21} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h21} and Rⁱ²¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j21} and R^{k21} is independently selected from OH, C₁₋₆ alkoxy, and C₁₋₆ haloalkoxy;
or any R^{j21} and R^{k21} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a22}, SR^{a22}, NHOR^{a22}, C(O)R²², C(O)NR^{c22}R^{d22}, C(O)NR^{c22}(OR^{a22}), C(O)OR^{a22}, OC(O)R²², OC(O)NR^{c22}R^{d22}, NR^{c22}R^{d22} NR R^{e2}R^{d22}, NR^{c22}C(O)R^{b22}, R^{c22}C(O)OR^{a22}, NR^{c22}C(O)NR^{c22}R^{d22}, C(═NR^{e22})R^{b22}, C(═NR^{e22})NR^{c22}R^{d22}, NR^{c22}C(═NR^{e22})NR^{c22}R^{d22}, NR^{c22}C(═NR^{e22})R^{b22}, NR^{c22}S(O)R^{b22}, NR^{c22}S(O)NR^{c22}R^{d22}, NR^{c22}S(O)₂R^{b22}, NR^{c22}S(O)(═NR^{e22})R^{b22}, NR^{c22}S(O)₂NR^{c22}R^{d22} S(O)R^{b22}, S(O)NR^{c22}R^{d22}, S(O)₂R^{b22}, S(O)₂NR^{c22}R^{d22} OS(O)(═NR^{e22})R^{b22}, OS(O)₂R^{b22}, SF₅, P(O)R^{f22}R^{g22}, OP(O)(OR^{h22})(ORⁱ²²), P(O)(OR^{h22})(ORⁱ²²) and BR^{j22}R^{k22}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{2B} are each optionally substituted with 1, 2, 3, 4, 5, 6, 7, or 8 independently selected R^{2C} substituents;
each R^{a22}, R^{c22}, and R^{d22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a22}, R^{c22} and R^{d22} are each optionally substituted with 1, 2, 3, 4, 5, 6, 7, or 8 independently selected R^{2C} substituents;
or, any R^{c22} and R^{d22} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{2C} substituents;
each R^{b22} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆-10 aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b22} are each optionally substituted with 1, 2, 3, 4, 5, 6, 7, or 8 independently selected R^{2C} substituents;
each R^{e22} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f22} and R^{g22} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h22} and Rⁱ²² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j22} and R^{k22} is independently selected from OH, C₁₋₆ alkoxy, and C₁₋₆ haloalkoxy;
or any R^{j22} and R^{k22} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{2C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a23}, SR^{a23}, NHOR^{a23}, C(O)R^{b23}, C(O)NR^{c23}R^{d23}, C(O)NR^{c23}(OR^{a23}), C(O)OR^{a23}, OC(O)R^{b23}, OC(O)NR^{c23}R^{d23}, NR^{c23}R^{d23}, NR^{c23}NR^{c23}R^{d23}, NR^{c23}C(O)R^{b23}, NR^{c23}C(O)OR^{a23}, NR^{c23}C(O)NR²³R^{d23}, C(═NR^{e23})R^{b23}, C(═NR^{e23})NR²³R^{d23}, NR^{e23}C(═NR^{e23})NR^{c23}R^{d23}, NR^{c23}C(═NR^{e23})R^{b23}, NR^{c23}S(O)R^{b23}, NR^{c23}S(O)NR^{c23}R^{d23}, NR^{c23}S(O)₂R^{b23}, NR^{c23}S(O)(═NR^{e23})R^{b23}, NR^{c23}S(O)₂NR^{c23}R^{d23}, S(O)R^{b23}, S(O)NR^{c23}R^{d23}, S(O)₂R^{b23}, S(O)₂NR^{c23}R^{d23}, OS(O)(═NR^{e23})R^{b23}, and OS(O)₂R^{b23}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{2C} are each optionally substituted with 1, 2, 3, or 4 independently selected R^{M} substituents;
each R^{a23}, R^{c23}, and R^{d23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a23}, R^{c23} and R^{d23} are each optionally substituted with 1, 2, 3, or 4 independently selected R^{M} substituents;
or, any R^{c23} and R^{d23} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b23} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b23} are each optionally substituted with 1, 2, 3, or 4 independently selected R^{M} substituents;
each R^{e23} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R³ is selected from H, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a3}, SR^{a3}, NHOR^{a3}, C(O)R^{b3}, C(O)NR^{c3}R^{d3}, C(O)NR^{c3}(OR^{a3}), C(O)OR^{a3}, OC(O)R^{b3}, OC(O)NR^{c3}R^{d3}, NR^{c3}R^{d3}, NR^{c3}NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}C(═NR^{e3})R^{b3}, NR^{c3}S(O)R^{b3}, NR^{c3}S(O)NR^{c3}R^{d3}, NR^{c3}S(O)₂R^{b3}, NR^{c3}S(O)(═NR^{e3})R^{b3}, NR^{c3}S(O)₂NR^{c3}R^{d3}, S(O)R^{b3}, S(O)NR^{c3}R^{d3}, S(O)₂R^{b3}, S(O)₂NR^{c3}R^{d3}, OS(O)(═NR^{e3})R^{b3}, OS(O)₂R^{b3}, SF₅, P(O)R^{f3}R^{g3}, OP(O)(OR^{h3})(ORⁱ³), P(O)(OR^{h3})(ORⁱ³), and BR^{j3}R^{k3}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, 4, 5, 6, 7, or 8 independently selected R^{3A} substituents;
each R^{a3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a3}, R^{c3} and R^{d3} are each optionally substituted with 1, 2, 3, 4, 5, 6, 7, or 8 independently selected R^{3A} substituents;
or, any R^{c3} and R^{d3} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3A} substituents;
each R^{b3} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b3} are each optionally substituted with 1, 2, 3, 4, 5, 6, 7, or 8 independently selected R^{3A} substituents;
each R^{e3} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f3} and R^{g3} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h3} and Rⁱ³ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j3} and R^{B3} is independently selected from OH, C₁₋₆ alkoxy, and C₁₋₆ haloalkoxy;
or any R^{j3} and R^{k3} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a31}, SR^{a31}, NHOR^{a31}, C(O)R^{b31}, C(O)NR^{c31}R^{d31}, C(O)NR^{c31}(OR^{a31}) C(O)OR^{a31}, OC(O)R^{b31}, OC(O)NR^{c31}R^{d31}, NR^{c31}R^{d31}, NR^{c31}NR^{c31}R^{d31}, NR^{c31}C(O)R^{b31}, NR^{c31}C(O)OR^{a31}, NR^{c31}(O)NR^{c31}R^{d31}, C(═NR^{e31})R^{b31}, C(═NR^{e31})NR^{c31}R^{d31}, NR^{c31}C(═NR^{e31})NR^{c31}R^{d31}, NR^{c31}C(═NR^{e11})R^{b31}, NR^{c31}S(O)R^{b31}, NR^{c31}S(O)NR^{c31}R^{d31}, NR^{c31}S(O)₂R^{b31}, NR^{c31}S(O)(═NR^{e31})R^{b31}, NR^{c31}S(O)₂NR^{c31}R^{d31}, S(O)R^{b31}, S(O)NR^{c31}R^{d31}, S(O)₂R^{b31}, S(O)₂NR^{c31}R^{d31}, OS(O)(═NR^{e31})R^{b31}, OS(O)₂R^{b31}, SF₅, P(O)R^{f31}R^{g31}, OP(O)(OR^{h31})(ORⁱ³¹), P(O)(OR^{h31})(ORⁱ³¹) and BR^{j31}R^{k31}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{3A} are each optionally substituted with 1, 2, 3, 4, 5, 6, 7, or 8 independently selected R^{3B} substituents;
each R^{a31}, R^{c31}, and R^{d31} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a}, R^{c31} and R^{d31} are each optionally substituted with 1, 2, 3, 4, 5, 6, 7, or 8 independently selected R^{3B} substituents;
or, any R^{c31} and R^{d31} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3B} substituents;
each R^{b31} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆-10 aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b31} are each optionally substituted with 1, 2, 3, 4, 5, 6, 7, or 8 independently selected R^{3B} substituents;
each R^{e31} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f31} and R^{g31} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h31} and Rⁱ³¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j31} and R^{k31} is independently selected from OH, C₁₋₆ alkoxy, and C₁₋₆ haloalkoxy;
or any R^{j31} and R^{k31} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a32}, SR^{a32}, NHOR^{a32}, C(O)R^{b32}, C(O)NR^{c32}R^{d32}, C(O)NR^{c32}(OR^{a32}), C(O)OR^{a32}, OC(O)R^{b32}, OC(O)NR^{c32}R^{d32}, NR^{c32}R^{d32}, NR^{c32}NR^{c32}R^{d32}, NR^{c32}C(O)R^{b32}, NR^{c32}C(O)OR^{a32}, NR^{c32}C(O)NR^{c32}R^{d32}, C(═NR^{e32})R^{b32}, C(═NR^{e32})NR^{c32}R^{d32}, NR^{c32}C(═NR^{e32}NR^{c32}R^{d32}, NR^{c32}C(═NR^{e32})R^{b32}, NR^{c32}S(O)R^{b32}, NR^{c32}S(O)NR^{c32}R^{d32}, NR^{c32}S(O)₂R^{b32}, NR^{c32}S(O)(═NR^{e32})R^{b32}, NR^{c32}S(O)₂NR^{c32}R^{d32}, S(O)R^{b32}, S(O)NR^{c32}R^{d32}, S(O)₂R^{b32}, S(O)₂NR^{c32}R^{d32} OS(O)(═N^{e32})R^{b32}, OS(O)₂R^{b32}, SF₅, P(O)R^{f32}R^{g32}, OP(O)(OR^{h32})(ORⁱ³²), P(O)(OR^{h32})(ORⁱ³²) and BR^{j32}R^{k32}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{3B} are each optionally substituted with 1, 2, 3, 4, 5, 6, 7, or 8 independently selected R^{3C} substituents;
each R^{a32}, R^{c32}, and R^{d32} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a32}, R^{c32} and R^{d32} are each optionally substituted with 1, 2, 3, 4, 5, 6, 7, or 8 independently selected R^{3C} substituents;
or, any R^{c32} and R^{d32} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{3C} substituents;
each R^{b32} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆-10 aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b32} are each optionally substituted with 1, 2, 3, 4, 5, 6, 7, or 8 independently selected R^{3C} substituents;
each R^{e32} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f32} and R^{g32} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h32} and Rⁱ³² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j32} and R^{k32} is independently selected from OH, C₁₋₆ alkoxy, and C₁₋₆ haloalkoxy;
or any R^{j32} and R^{k32} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{3C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a33}, SR^{a33}, NHOR^{a33}, C(O)R^{b33}, C(O)NR^{c33}R^{d33}, C(O)NR^{c33}(OR^{a33}), C(O)OR^{a33}, OC(O)R^{b33}, OC(O)NR^{c33}R^{d33}, NR^{c33}R^{d33}, NR^{c33}NR^{c33}R^{d33}, NR^{c33}C(O)R^{b33}, NR^{c33}C(O)OR^{a33}, NR^{c33}C(O)NR^{c33}R^{d33}, C(═NR^{e33})R^{b33}, C(═NR^{e33})NR^{c33}R^{d33}, NR^{c33}C(═NR^{e33})NR^{c33}R^{d33}, NR^{c33}C(═NR^{e33})R^{b33}, NR^{c33}S(O)R^{b33}, NR^{c33}S(O)NR^{c33}R^{d33}, NR^{c33}S(O)₂R^{b33}, NR^{c33}S(O)(═NR^{e33})R^{b33}, NR^{c33}S(O)₂NR^{c33}R^{d33}, S(O)R^{b33}, S(O)NR^{c33}R^{d33}, S(O)₂R^{b33}, S(O)₂NR^{c33}R^{d33}, OS(O)(═NR^{e33})R^{b33}, and OS(O)₂R^{b33}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{3C} are each optionally substituted with 1, 2, 3, or 4 independently selected R^{M} substituents;
each R^{a33}, R^{c33}, and R^{d33} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a3}, R^{c33} and R^{d33} are each optionally substituted with 1, 2, 3, or 4 independently selected R^{M} substituents;
or, any R^{c33} and R^{d33} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b33} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b33} are each optionally substituted with 1, 2, 3, or 4 independently selected R^{M} substituents;
each R^{e33} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
R⁴ is selected from C₁₋₆ alkylene, C₁₋₆ haloalkylene, C₂₋₆ alkenylene, C₂₋₆ alkynylene, C₆₋₁₀ arylene, C₃₋₁₀ cycloalkylene, 5-10 membered heteroarylene, 4-10 membered heterocycloalkylene, —C₆₋₁₀ arylene-C₁₋₆ alkylene-, —C₃₋₁₀ cycloalkylene-C₁₋₆ alkylene-, -(5-10 membered heteroarylene)-C₁₋₆ alkylene-, and -(4-10 membered heterocycloalkylene)-C₁₋₆ alkylene-, wherein the C₁₋₆ alkylene, C₂₋₆ alkenylene, C₂₋₆ alkynylene, C₆₋₁₀ arylene, C₃₋₁₀ cycloalkylene, 5-10 membered heteroarylene, 4-10 membered heterocycloalkylene, —C₆₋₁₀ aryl-C₁₋₆ alkylene-, —C₃₋₁₀ cycloalkyl-C₁₋₆ alkylene-, -(5-10 membered heteroaryl)-C₁₋₆ alkylene-, and -(4-10 membered heterocycloalkyl)-C₁₋₆ alkylene- of R⁴ are each optionally substituted with 1, 2, 3, 4, 5, 6, 7, or 8 independently selected R^{4A} substituents;
each R^{4A} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a41}, SR^{a41}, NHOR^{a41}C(O)R^{b41}, C(O)NR^{c41}R^{d41}, C(O)NR^{c41}(OR^{a41}), C(O)OR^{a41}, OC(O)R^{b41}, OC(O)NR^{c41}R^{d41}, NR^{c41}R^{d41}, NR^{c41}NR^{c41}R^{d41}, NR^{c41}C(O)R^{b41}, NR^{c41}C(O)OR^{a41}, NR^{c41}C(O)NR^{c41}R^{d41}, C(═NR^{e41})R^{b41}, C(═NR^{e41})NR^{c41}R^{d41}, NR^{c41}C(═NR^{e41})NR^{c41}R^{d41}, NR^{c41}C(═NR^{e41})R^{b41}, NR^{c41}S(O)R^{b41}, NR^{c41}S(O)NR^{c41}R^{d41}, NR^{c41}S(O)₂R^{b41}, NR^{c41}S(O)(═NR^{e41})R^{b41}, NR^{c41}S(O)₂NR^{c41}R^{d41}, S(O)R^{b41}, S(O)NR^{c41}R^{d41}, S(O)₂R^{b41}, S(O)₂NR^{c41}R^{d41} OS(O)(═NR^{e41})R^{b41}, OS(O)₂R^{b41}, SF₅, P(O)R^{f41}R^{g41}, OP(O)(OR^{h41})(ORⁱ⁴¹), P(O)(OR^{h41})(ORⁱ⁴¹), and BR^{j41}R^{k41}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{4A} are each optionally substituted with 1, 2, 3, 4, 5, 6, 7, or 8 independently selected R^{4B} substituents;
each R^{a41}, R^{c41}, and R^{d41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a41}, R^{c41} and R^{d41} are each optionally substituted with 1, 2, 3, 4, 5, 6, 7, or 8 independently selected R^{4B} substituents;
or, any R^{c41} and R^{d41} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4B} substituents;
each R^{b41} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆-10 aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b41} are each optionally substituted with 1, 2, 3, 4, 5, 6, 7, or 8 independently selected R^{4B} substituents;
each R^{e41} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f41} and R^{g41} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h41} and Rⁱ⁴¹ is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j41} and R^{k41} is independently selected from OH, C₁₋₆ alkoxy, and C₁₋₆ haloalkoxy;
or any R^{j41} and R^{k41} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4B} is independently selected from halo, oxo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, (4-10 membered heterocycloalkyl)-C₁₋₆ alkyl-, CN, NO₂, OR^{a42}, SR^{a42}, NHOR^{a42}C(O)R^{b42}, C(O)NR^{c42}R^{d42}, C(O)NR^{c42}(OR^{a42}), C(O)OR^{a42}, OC(O)R^{b42}, OC(O)NR^{c42}R^{d42}, NR^{c42}R^{d42}, NR^{c42}NR^{c42}R^{d42}, NR^{c42}C(O)R^{b42}, NR^{c42}C(O)OR^{a42}, NR^{c42}C(O)NR^{c42}R^{d42}, C(═NR^{e42})R^{b42}, C(═NR^{e42})NR^{c42}R^{d42}, NR^{c42}C(═NR^{e42})NR^{c42}R^{d42}, NR^{c42}C(═NR^{e42})R^{b42}, NR^{c42}S(O)R^{b42}, NR^{c42}S(O)NR^{c42}R^{d42}, NR^{c42}S(O)₂R^{b42}, NR^{c42}S(O)(═NR^{e42})R^{b42}, NR^{c42}S(O)₂NR^{c42}R^{d42} S(O)R^{b42}, S(O)NR^{c42}R^{d42}, S(O)₂R^{b42}, S(O)₂NR^{c42}R^{d42} OS(O)(═NR^{e42})R^{b42}, OS(O)₂R^{b42}, SF₅, P(O)R^{f42}R^{g42}, OP(O)(OR^{h42})(ORⁱ⁴²), P(O)(OR^{h42})(ORⁱ⁴²), and BR^{j42}R^{k42}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{4B} are each optionally substituted with 1, 2, 3, 4, 5, 6, 7, or 8 independently selected R^{4C} substituents;
each R^{a42}, R^{c42}, and R^{d42} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{a42}, R^{c42} and R^{d42} are each optionally substituted with 1, 2, 3, 4, 5, 6, 7, or 8 independently selected R^{4C} substituents;
or, any R^{c42} and R^{d42} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, or 4 independently selected R^{4C} substituents;
each R^{b42} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₁₋₆ alkyl, C₂₋₆ alkenyl, C₂₋₆ alkynyl, C₆-10 aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R^{b42} are each optionally substituted with 1, 2, 3, 4, 5, 6, 7, or 8 independently selected R^{4C} substituents;
each R^{e42} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{f42} and R^{g42} is independently selected from H, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{h42} and Rⁱ⁴² is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-;
each R^{j42} and R^{k42} is independently selected from OH, C₁₋₆ alkoxy, and C₁₋₆ haloalkoxy;
or any R^{j42} and R^{k42} attached to the same B atom, together with the B atom to which they are attached, form a 5- or 6-membered heterocycloalkyl group optionally substituted with 1, 2, 3, or 4 substituents independently selected from C₁₋₆ alkyl and C₁₋₆ haloalkyl;
each R^{4C} is independently selected from halo, oxo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, (4-7 membered heterocycloalkyl)-C₁₋₆ alkyl-, CN, NO₂, OR^{a43}, SR^{a43}, NHOR^{a43}, C(O)R⁴³, C(O)NR^{c43}R^{d43}, C(O)NR^{c43}(OR^{a43}), C(O)OR^{a43}, OC(O)R⁴³, OC(O)NR^{c43}R^{d43}, NR^{c43}R^{d43}, NR^{c43}NR^{c43}R^{d43}, NR^{c43}C(O)R^{b43}, NR^{c43}C(O)OR^{a43}, NR^{c43}C(O)NR^{c43}R^{d43}, C(═NR^{e43})R^{b43}, C(═NR^{e43})NR^{c43}R^{d43}, NR^{c43}C(═NR^{e43})NR^{c43}R^{d43}, NR^{c43}C(═NR^{e43})R^{b43}, NR^{c43}S(O)R^{b43}, NR^{c43}S(O)NR^{c43}R^{d43}, NR^{c43}S(O)₂R^{b43}, NR⁴³S(O)(═NR^{e43})R^{b43}, NR^{c43}S(O)₂NR^{c43}R^{d43}, S(O)R^{b43}, S(O)NR^{c43}R^{d43}, S(O)₂R^{b43}, S(O)₂NR^{c43}R^{d43}, OS(O)(═NR^{e43})R^{b43}, and OS(O)₂R^{b43}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{4C} are each optionally substituted with 1, 2, 3, or 4 independently selected R^{M} substituents;
each R^{a43}, R^{c43}, and R^{d43} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{a43}, R^{c43} and R^{d43} are each optionally substituted with 1, 2, 3, or 4 independently selected R^{M} substituents;
or, any R^{c43} and R^{d43} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b43} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl- of R^{b43} are each optionally substituted with 1, 2, 3, or 4 independently selected R^{M} substituents;
each R^{c43} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkyl-, C₃₋₇ cycloalkyl-C₁₋₆ alkyl-, (5-6 membered heteroaryl)-C₁₋₆ alkyl-, and (4-7 membered heterocycloalkyl)-C₁₋₆ alkyl-;
X¹ is selected from —CH₂—, C(O), and —CH₂C(O)—;
L is selected from —N(R^{N1})—R⁵—N(R^{N2})—, —N(R^{N1})—R⁵—O— 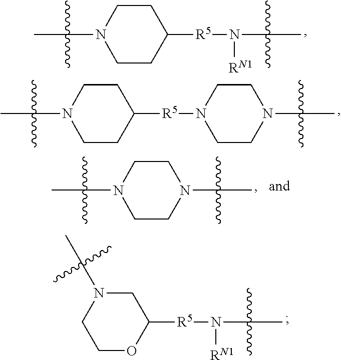
R^{N1} is H or C₁₋₆ alkyl;
R^{N2} is H or C₁₋₆ alkyl;
R⁵ is selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ haloalkylene-O—C₁₋₆ alkylene-C(O)—, —C₁₋₆ alkylene-C₃₋₇ cycloalkylene-C(O)—, and —C₁₋₆ alkylene-phenyl-C(O)—;
E is selected from: 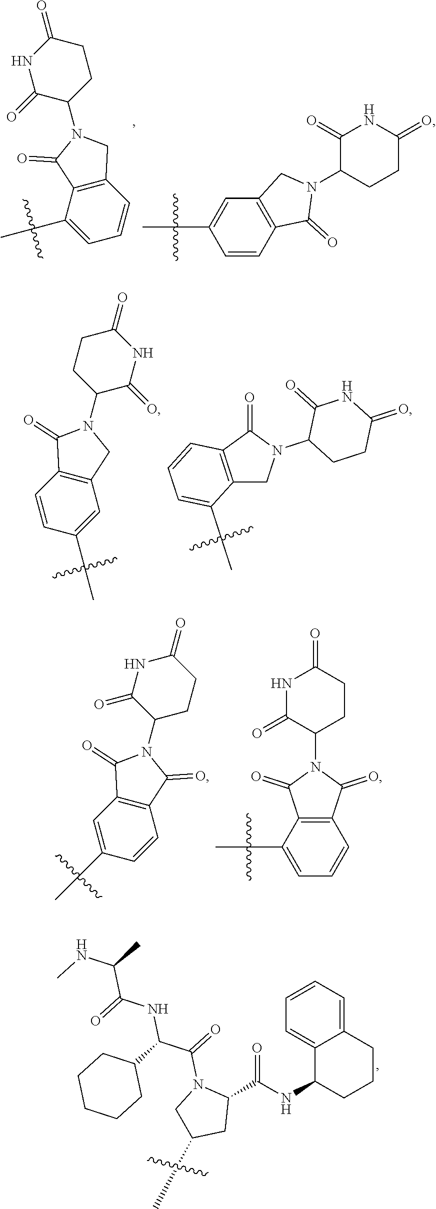 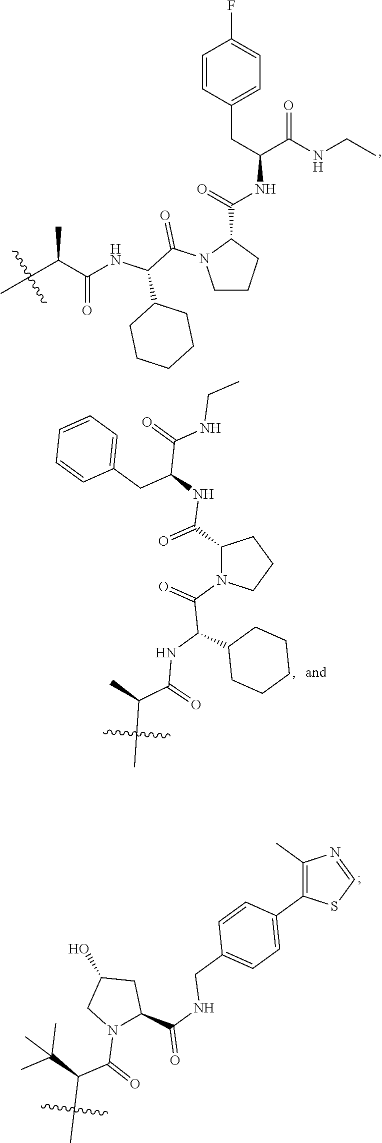
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-.

2. The compound of claim 1, or a pharmaceutically acceptable salt thereof, wherein R¹ is selected from H, and C₁₋₆ alkyl.

3. The compound of claim 1, or a pharmaceutically acceptable salt thereof, wherein R¹ is C₁₋₆ alkyl.

4. The compound of claim 1, or a pharmaceutically acceptable salt thereof, wherein R¹ is methyl.

5. The compound of claim 1, or a pharmaceutically acceptable salt thereof, wherein R² is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents.

6. The compound of claim 1, or a pharmaceutically acceptable salt thereof, wherein R² is selected from phenyl, C₃₋₇ cycloalkyl, 5-6 membered heteroaryl, and 4-7 membered heterocycloalkyl, wherein the phenyl, C₃₋₇ cycloalkyl, 5-6 membered heteroaryl, and 4-7 membered heterocycloalkyl of R² are each optionally substituted with 1, 2, 3, or 4 independently selected R^{2A} substituents.

7. The compound of claim 1, or a pharmaceutically acceptable salt thereof, wherein R² is C₃₋₇ cycloalkyl, which is optionally substituted with 1, 2, 3, or 4 independently selected R^{2A} substituents.

8. The compound of claim 1, or a pharmaceutically acceptable salt thereof, wherein R² is cyclopentyl, which is optionally substituted with 1, 2, 3, or 4 independently selected R^{2A} substituents.

9. The compound of claim 1, or a pharmaceutically acceptable salt thereof, wherein each R^{2A} is independently selected from NR^{c21}C(O)R^{b21}, NR^{c21}C(O)OR^{a21}, and NR^{c21}C(O)NR^{c21}R^{d21}; and
each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl.

10. The compound of claim 1, or a pharmaceutically acceptable salt thereof, wherein each R^{2A} is independently selected from NR^{c21}C(O)OR^{a21}; and
each R^{a21} and R^{c21} is independently selected from H and C₁₋₆ alkyl.

11. The compound of claim 1, or a pharmaceutically acceptable salt thereof, wherein each R^{2A} is NHC(O)OCH₃.

12. The compound of claim 1, or a pharmaceutically acceptable salt thereof, wherein R³ is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, or 4 independently selected R^{3A} substituents.

13. The compound of claim 1, or a pharmaceutically acceptable salt thereof, wherein R³ is selected from phenyl, 8-10 membered heteroaryl, and 8-10 membered heterocycloalkyl, wherein the phenyl, 8-10 membered heteroaryl, and 8-10 membered heterocycloalkyl of R³ are each optionally substituted with 1, 2, 3, or 4 independently selected R^{3A} substituents.

14. The compound of claim 1, or a pharmaceutically acceptable salt thereof, wherein R³ is selected from phenyl, indazolyl, and dihydroisobenzofuranyl, wherein the phenyl, indazolyl, and dihydroisobenzofuranyl of R³ are each optionally substituted with 1, 2, 3, or 4 independently selected R^{3A} substituents.

15. The compound of claim 1, or a pharmaceutically acceptable salt thereof, wherein R³ is selected from

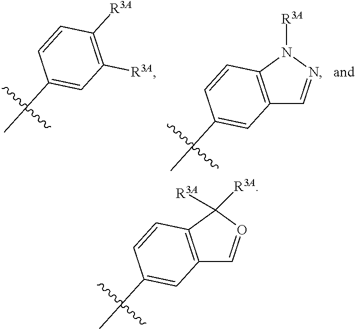

16. The compound of claim 1, or a pharmaceutically acceptable salt thereof, wherein each R^{3A} is independently selected from halo, oxo, C₁₋₆ alkyl, C₁₋₆ haloalkyl, C₂₋₆ alkenyl, C₂₋₆ alkynyl, and OR^{a31}; and
each R^{a31} is independently selected from H, C₁₋₆ alkyl, C₁₋₆ haloalkyl, C₂₋₆ alkenyl, and C₂₋₆ alkynyl.

17. The compound of claim 1, or a pharmaceutically acceptable salt thereof, wherein each R^{3A} is independently selected from halo, C₁₋₆ alkyl, C₁₋₆ haloalkyl, and OR^{a31}; and
each R^{a31} is independently selected from H and C₁₋₆ alkyl.

18. The compound of claim 1, or a pharmaceutically acceptable salt thereof, wherein each R^{3A} is independently selected from methyl, isopropyl, fluoro, and methoxy.

19. The compound of claim 1, or a pharmaceutically acceptable salt thereof, wherein R³ is selected from

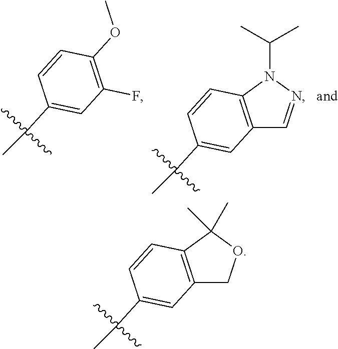

20. The compound of claim 1, wherein R⁴ is selected from C₆₋₁₀ arylene, C₃₋₁₀ cycloalkylene, 5-10 membered heteroarylene, and 4-10 membered heterocycloalkylene, wherein the C₆₋₁₀ arylene, C₃₋₁₀ cycloalkylene, 5-10 membered heteroarylene, and 4-10 membered heterocycloalkylene of R⁴ are each optionally substituted with 1, 2, 3, or 4 independently selected R^{4A} substituents.

21. The compound of claim 1, or a pharmaceutically acceptable salt thereof, wherein R⁴ is selected from phenylene and 5-6 membered heteroarylene, wherein the phenylene and 5-6 membered heteroarylene of R⁴ are each optionally substituted with 1, 2, 3, or 4 independently selected R^{4A} substituents.

22. The compound of claim 1, or a pharmaceutically acceptable salt thereof, wherein R⁴ is selected from

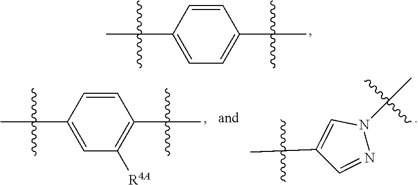

23. The compound of claim 1, or a pharmaceutically acceptable salt thereof, wherein each R^{4A} is independently selected from halo, C₁₋₆ alkyl, C₁₋₆ haloalkyl, C₂₋₆ alkenyl, C₂₋₆ alkynyl, and OR^{a41}; and
each R^{a41} is independently selected from H, C₁₋₆ alkyl, C₁₋₆ haloalkyl, C₂₋₆ alkenyl, and C₂₋₆ alkynyl.

24. The compound of claim 1, or a pharmaceutically acceptable salt thereof, wherein each R^{4A} is independently selected from halo and OR^{a41}; and
each R^{a41} is independently selected from H and C₁₋₆ alkyl.

25. The compound of claim 1, or a pharmaceutically acceptable salt thereof, wherein each R^{4A} is independently selected from fluoro and methoxy.

26. The compound of claim 1, or a pharmaceutically acceptable salt thereof, wherein R⁴ is selected from

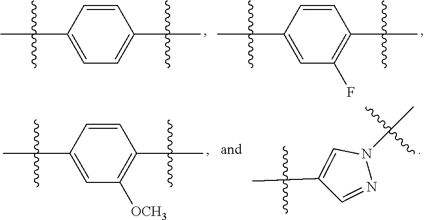 [SMILES: *c1ccc(*)cc1 ; *c1ccc(*)c(F)c1 ; *c1ccc(*)c(OC)c1 ; *c1cnn(*)c1]

27. The compound of claim 1, or a pharmaceutically acceptable salt thereof, wherein X¹ is —CH₂—.

28. The compound of claim 1, or a pharmaceutically acceptable salt thereof, wherein X¹ is C(O).

29. The compound of claim 1, or a pharmaceutically acceptable salt thereof, wherein X¹ is —CH₂C(O)—.

30. The compound of claim 1, or a pharmaceutically acceptable salt thereof, wherein R^{N1} is selected from H and methyl.

31. The compound of claim 1, or a pharmaceutically acceptable salt thereof, wherein R^{N2} is selected from H and methyl.

32. The compound of claim 1, or a pharmaceutically acceptable salt thereof, wherein R^{N2} is H.

33. The compound of claim 1, or a pharmaceutically acceptable salt thereof, wherein R⁵ is selected from —C(O)—, —CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂C(O)—, —CH₂CH₂CH₂CH₂CH₂CH₂C(O)—, —CH₂CH₂CH₂CH₂—O—CH₂CF₂CH₂—O—CH₂C(O)—, —CH₂CH₂—O—CH₂CH₂—, —CH₂CH₂—O—CH₂CH₂—O—CH₂C(O)—, —CH₂CH₂—O—CH₂CH₂—O—CH₂CH₂C(O)—, —CH₂CH₂—O—CH₂C(O)—, —CH₂CH₂—O—CH₂CH₂C(O)—,

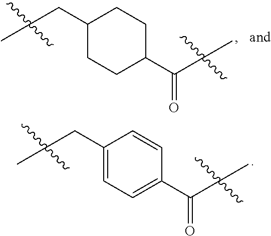

34. The compound of claim 1, or a pharmaceutically acceptable salt thereof, wherein L is selected from:

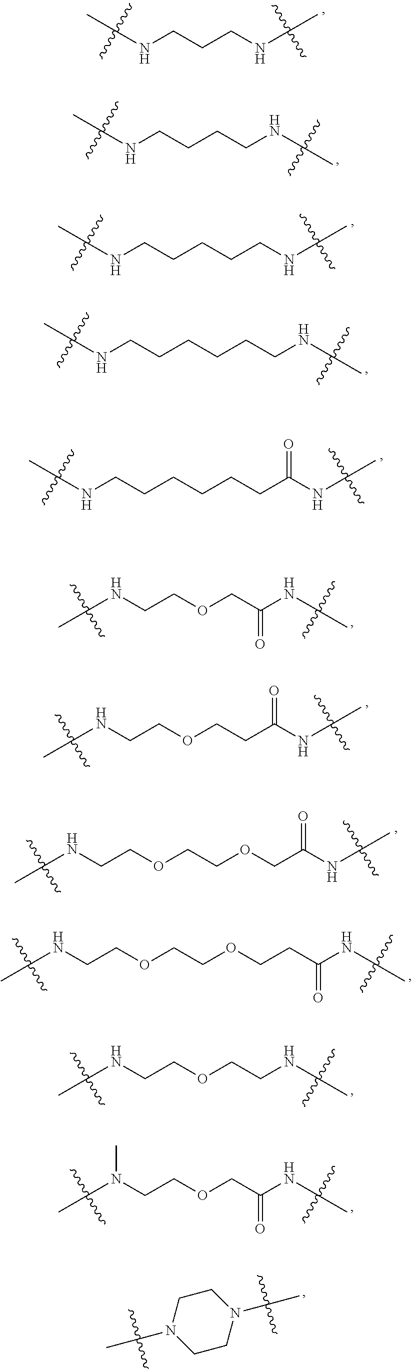

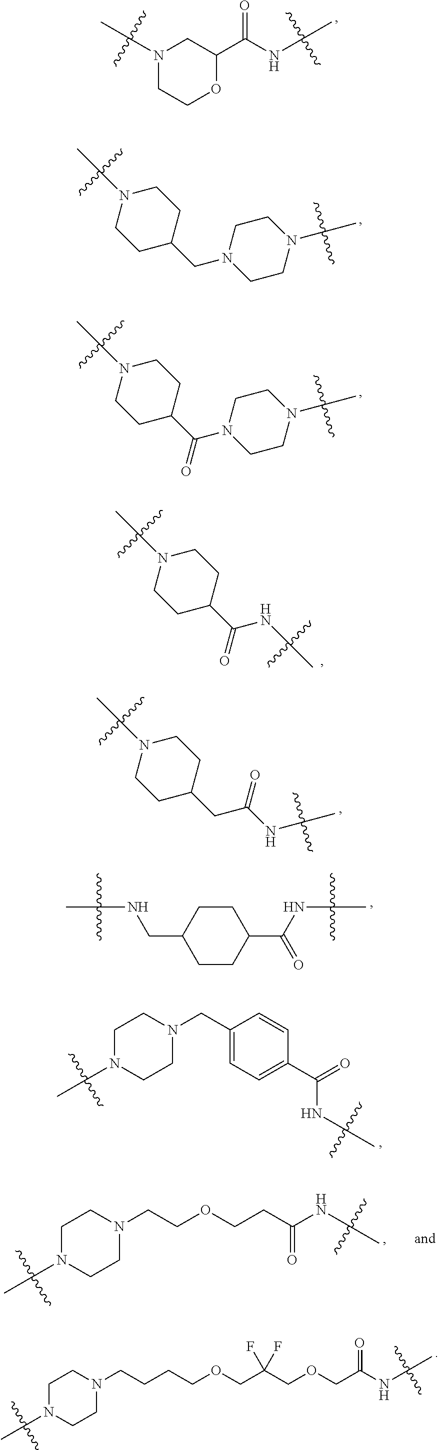

35. The compound of claim 1, or a pharmaceutically acceptable salt thereof, wherein:
R¹ is selected from H, C₁₋₆ alkyl, C₂₋₆ alkenyl, and C₂₋₆ alkynyl, wherein the C₁₋₆ alkyl, C₂₋₆ alkenyl, and C₂₋₆ alkynyl;
R² is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R² are each optionally substituted with 1, 2, 3, or 4 independently selected R^{2A} substituents;
each R^{2A} is independently selected from NR^{c21}C(O)R^{b21}, NR^{c21}C(O)OR^{a21}, and NR^{c21}C(O)NR^{c21}R^{d21};
each R^{a21}, R^{b21}, R^{c21}, and R^{d21} is independently selected from H, C₁₋₆ alkyl, C₁₋₆ haloalkyl, C₂₋₆ alkenyl, and C₂₋₆ alkynyl;
R³ is selected from C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl-, wherein the C₆₋₁₀ aryl, C₃₋₁₀ cycloalkyl, 5-10 membered heteroaryl, 4-10 membered heterocycloalkyl, C₆₋₁₀ aryl-C₁₋₆ alkyl-, C₃₋₁₀ cycloalkyl-C₁₋₆ alkyl-, (5-10 membered heteroaryl)-C₁₋₆ alkyl-, and (4-10 membered heterocycloalkyl)-C₁₋₆ alkyl- of R³ are each optionally substituted with 1, 2, 3, or 4 independently selected R^{3A} substituents;
each R^{3A} is independently selected from halo, oxo, C₁₋₆ alkyl, C₁₋₆ haloalkyl, C₂₋₆ alkenyl, C₂₋₆ alkynyl, and OR^{a31};
each R^{a31} is independently selected from H, C₁₋₆ alkyl, C₁₋₆ haloalkyl, C₂₋₆ alkenyl, and C₂₋₆ alkynyl;
R⁴ is selected from C₆₋₁₀ arylene, C₃₋₁₀ cycloalkylene, 5-10 membered heteroarylene, and 4-10 membered heterocycloalkylene, wherein the C₆₋₁₀ arylene, C₃₋₁₀ cycloalkylene, 5-10 membered heteroarylene, and 4-10 membered heterocycloalkylene of R⁴ are each optionally substituted with 1, 2, 3, or 4 independently selected R^{4A} substituents;
each R^{4A} is independently selected from halo, C₁₋₆ alkyl, C₁₋₆ haloalkyl, C₂₋₆ alkenyl, C₂₋₆ alkynyl, and OR^{a41};
each R^{a41} is independently selected from H, C₁₋₆ alkyl, C₁₋₆ haloalkyl, C₂₋₆ alkenyl, and C₂₋₆ alkynyl;
X¹ is selected from —CH₂—, C(O), and —CH₂C(O)—;
L is selected from —N(R^{N1})—R⁵—N(R^{N2})—, —N(R^{N1})—R⁵—O—, 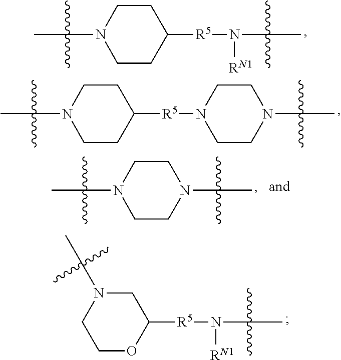
R^{N1} is H or C₁₋₃ alkyl;
R^{N2} is H or C₁₋₃ alkyl;
R⁵ is selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ haloalkylene-O—C₁₋₆ alkylene-C(O)—, —C₁₋₆ alkylene-C₃₋₇ cycloalkylene-C(O)—, and —C₁₋₆ alkylene-phenyl-C(O)—; and
E is selected from:

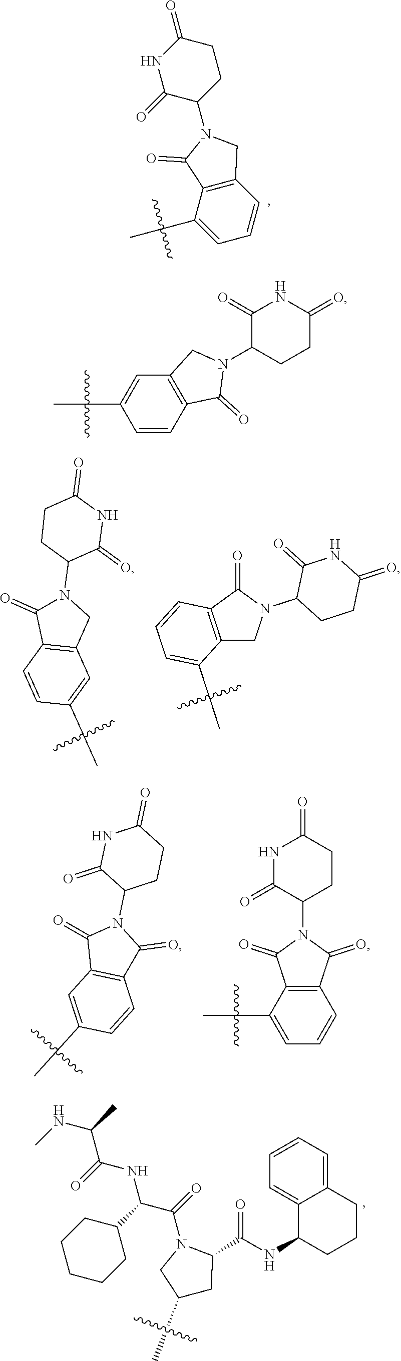

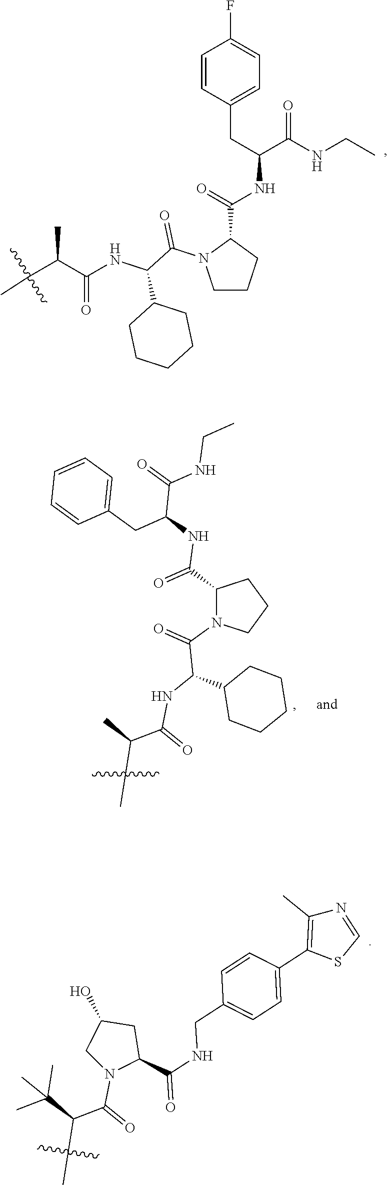

36. The compound of claim 1, or a pharmaceutically acceptable salt thereof, wherein:
R¹ is C₁₋₆ alkyl;
R² is C₃₋₇ cycloalkyl, which is optionally substituted with 1, 2, 3, or 4 independently selected R^{2A} substituents;
each R^{2A} is independently selected from NR^{c21}C(O)OR^{a21};
each R^{a21} and R^{c21} is independently selected from H and C₁₋₆ alkyl;
R³ is selected from phenyl, 8-10 membered heteroaryl, and 8-10 membered heterocycloalkyl, wherein the phenyl, 8-10 membered heteroaryl, and 8-10 membered heterocycloalkyl of R³ are each optionally substituted with 1, 2, 3, or 4 independently selected R^{3A} substituents;
each R^{3A} is independently selected from halo, C₁₋₆ alkyl, C₁₋₆ haloalkyl, and OR^{a31};
each R^{a31} is independently selected from H and C₁₋₆ alkyl;
R⁴ is selected from phenylene and 5-6 membered heteroarylene, wherein the phenylene and 5-6 membered heteroarylene of R⁴ are each optionally substituted with 1, 2, 3, or 4 independently selected R^{4A} substituents;
each R^{4A} is independently selected from halo and OR^{a41};
each R^{a31} is independently selected from H and C₁₋₆ alkyl;
X¹ is selected from —CH₂—, C(O), and —CH₂C(O)—;
L is selected from —N(R^{N1})—R⁵—N(R^{N2})—, —N(R^{N1})—R⁵—O—, 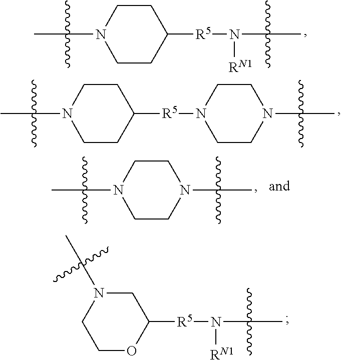
R^{N1} is H or C₁₋₃ alkyl;
R^{N2} is H or C₁₋₃ alkyl;
R⁵ is selected from C(O), C₁₋₈ alkylene, C₁₋₈ alkylene-C(O)—, —C₁₋₆ alkylene-O—C₁₋₆ alkylene-, —C₁₋₆ alkylene-O—C₁₋₆ alkylene-C(O)—, —C₁₋₆ alkylene-O—C₁₋₆ alkylene-O—C₁₋₆ alkylene-C(O)—, —C₁₋₆ alkylene-O—C₁₋₆ haloalkylene-O—C₁₋₆ alkylene-C(O)—, —C₁₋₆ alkylene-C₃₋₇ cycloalkylene-C(O)—, and —C₁₋₆ alkylene-phenyl-C(O)—; and
E is selected from:

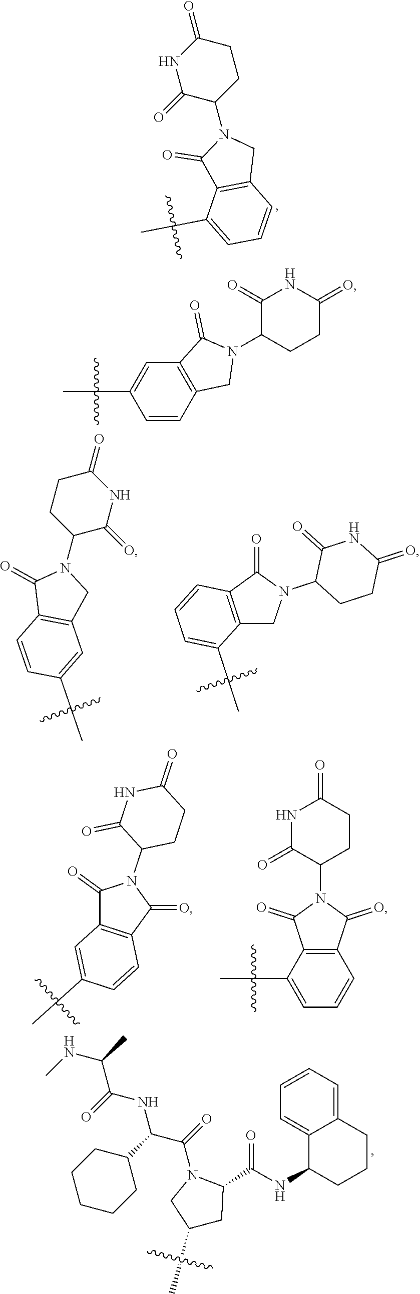

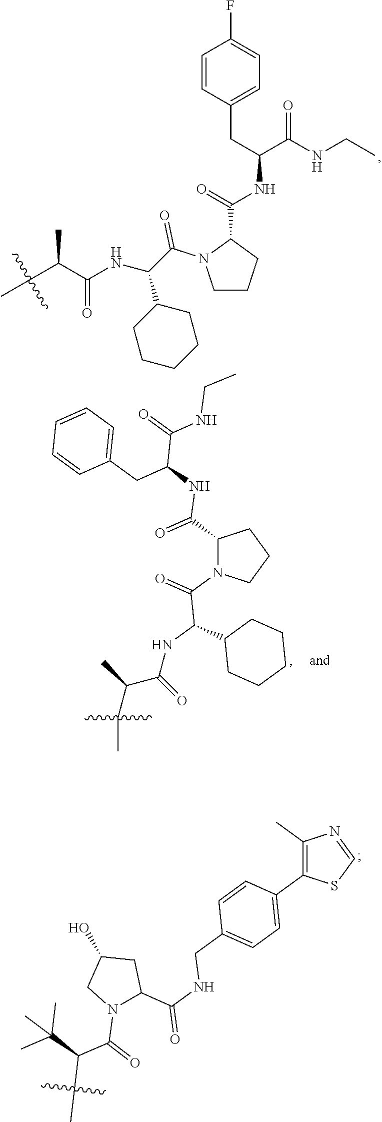

37. The compound of claim 1, wherein the compound of Formula I is a compound of Formula II: 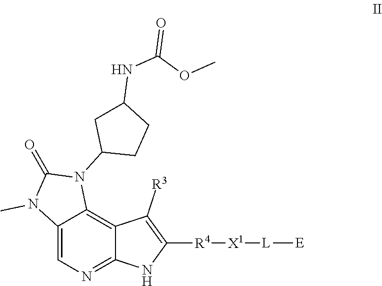
or a pharmaceutically acceptable salt thereof.

38. The compound of claim 1, wherein the compound of Formula I is a compound of Formula III: 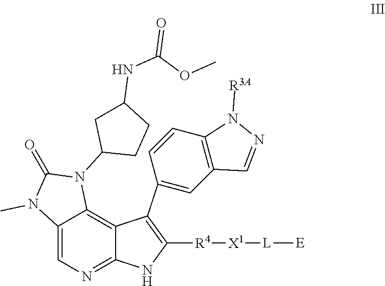
or a pharmaceutically acceptable salt thereof.

39. The compound of claim 1, wherein the compound of Formula I is a compound of Formula IV: 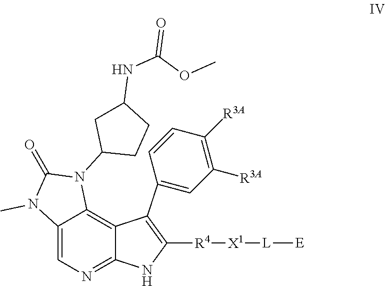
or a pharmaceutically acceptable salt thereof.

40. The compound of claim 1, wherein the compound of Formula I is a compound of Formula IV: 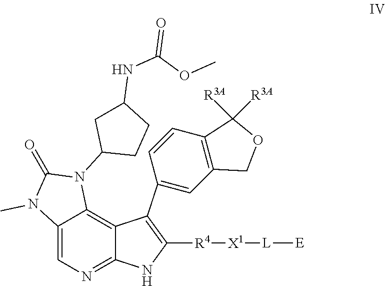
or a pharmaceutically acceptable salt thereof.

41. The compound of claim 1, which is selected from:
methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((3-((2-(2,6-dioxopiperidin-3-yl)-1,3-dioxoisoindolin-5-yl)amino)propyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((2-(2,6-dioxopiperidin-3-yl)-1,3-dioxoisoindolin-5-yl)amino)butyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((5-((2-(2,6-dioxopiperidin-3-yl)-1,3-dioxoisoindolin-5-yl)amino)pentyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((4-((2-(2,6-dioxopiperidin-3-yl)-1,3-dioxoisoindolin-5-yl)amino)butyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1,3-dioxoisoindolin-4-yl)amino)-2-oxoethoxy)ethyl)carbamoyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((3-((2-(2,6-dioxopiperidin-3-yl)-1,3-dioxoisoindolin-5-yl)amino)propyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((5-((2-(2,6-dioxopiperidin-3-yl)-1,3-dioxoisoindolin-5-yl)amino)pentyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(8-(1,1-dimethyl-1,3-dihydroisobenzofuran-5-yl)-7-(4-((2-(2-((2-(2,6-dioxopiperidin-3-yl)-1-oxoisoindolin-5-yl)amino)-2-oxoethoxy)ethyl)carbamoyl)-3-fluorophenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((2-(2,6-dioxopiperidin-3-yl)-1,3-dioxoisoindolin-5-yl)amino)butyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((5-((2-(2,6-dioxopiperidin-3-yl)-1,3-dioxoisoindolin-5-yl)amino)pentyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(3-fluoro-4-((2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(3-fluoro-4-((2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)ethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-(3-((2-(2,6-dioxopiperidin-3-yl)-1-oxoisoindolin-5-yl)amino)-3-oxopropoxy)ethyl)piperazine-1-carbonyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-((2-(2,6-dioxopiperidin-3-yl)-1-oxoisoindolin-5-yl)carbamoyl)benzyl)piperazine-1-carbonyl)-3-fluorophenyl)-8-(3-fluoro-4-methoxyphenyl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-((2-(2,6-dioxopiperidin-3-yl)-1-oxoisoindolin-5-yl)carbamoyl)benzyl)piperazine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((7-((2-(2,6-dioxopiperidin-3-yl)-1-oxoisoindolin-5-yl)amino)-7-oxoheptyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-((2-(2,6-dioxopiperidin-3-yl)-1,3-dioxoisoindolin-4-yl)carbamoyl)benzyl)piperazine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1,3-dioxoisoindolin-4-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-(3-(2-((2-(2,6-dioxopiperidin-3-yl)-1,3-dioxoisoindolin-4-yl)amino)-2-oxoethoxy)-2,2-difluoropropoxy)butyl)piperazine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-3-oxoisoindolin-4-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-4-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-3-oxoisoindolin-5-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1,3-dioxoisoindolin-5-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)(methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(((S)-1-(((S)-1-cyclohexyl-2-((S)-2-(((S)-1-(ethylamino)-1-oxo-3-phenylpropan-2-yl)carbamoyl)pyrrolidin-1-yl)-2-oxoethyl)amino)-1-oxopropan-2-yl)amino)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((6-(((S)-1-(((S)-1-cyclohexyl-2-((S)-2-(((S)-1-(ethylamino)-1-oxo-3-phenylpropan-2-yl)carbamoyl)pyrrolidin-1-yl)-2-oxoethyl)amino)-1-oxopropan-2-yl)amino)hexyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(((S)-1-(((S)-1-cyclohexyl-2-((S)-2-(((S)-1-(ethylamino)-3-(4-fluorophenyl)-1-oxopropan-2-yl)carbamoyl)pyrrolidin-1-yl)-2-oxoethyl)amino)-1-oxopropan-2-yl)amino)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-5-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-4-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1,3-dioxoisoindolin-5-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1-oxoisoindolin-5-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((4-(2-(2,6-dioxopiperidin-3-yl)-1-oxoisoindolin-5-yl)piperazin-1-yl)methyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(4-(2-(2,6-dioxopiperidin-3-yl)-1,3-dioxoisoindolin-4-yl)piperazine-1-carbonyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((1-(2-(2,6-dioxopiperidin-3-yl)-1,3-dioxoisoindolin-4-yl)piperidin-4-yl)methyl)piperazine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((2-(2,6-dioxopiperidin-3-yl)-1,3-dioxoisoindolin-4-yl)amino)butyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(1-(2-(4-((4-(2-(2,6-dioxopiperidin-3-yl)-1,3-dioxoisoindolin-4-yl)piperazin-1-yl)methyl)piperidin-1-yl)-2-oxoethyl)-1H-pyrazol-4-yl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(1-(2-(4-((4-(2-(2,6-dioxopiperidin-3-yl)-1-oxoisoindolin-5-yl)piperazin-1-yl)methyl)piperidin-1-yl)-2-oxoethyl)-1H-pyrazol-4-yl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((1-(2-(2,6-dioxopiperidin-3-yl)-1,3-dioxoisoindolin-4-yl)piperidin-4-yl)methyl)piperazin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((4-(2-(2,6-dioxopiperidin-3-yl)-1-oxoisoindolin-5-yl)piperazin-1-yl)methyl)piperidin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((4-((4-(2-(2,6-dioxopiperidin-3-yl)-1,3-dioxoisoindolin-4-yl)piperazin-1-yl)methyl)piperidin-1-yl)methyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans)-4-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)carbamoyl)cyclohexyl)methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans-)-4-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)carbamoyl)cyclohexyl)methyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(3-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-3-oxopropoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(2-(((3S,5S)-1-((S)-2-cyclohexyl-2-((S)-2-(methylamino)propanamido)acetyl)-5-(((R)-1,2,3,4-tetrahydronaphthalen-1-yl)carbamoyl)pyrrolidin-3-yl)amino)-2-oxoethoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-(3-((2-(2,6-dioxopiperidin-3-yl)-1-oxoisoindolin-5-yl)amino)-3-oxopropoxy)ethoxy)ethyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-((2-(2,6-dioxopiperidin-3-yl)-1-oxoisoindolin-5-yl)amino)-2-oxoethyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans-)-4-((2-(2,6-dioxopiperidin-3-yl)-1-oxoisoindolin-5-yl)carbamoyl)cyclohexyl)methyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((((trans)-4-((2-(2,6-dioxopiperidin-3-yl)-1-oxoisoindolin-5-yl)carbamoyl)cyclohexyl)methyl)carbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(2-((2-(2,6-dioxopiperidin-3-yl)-1-oxoisoindolin-5-yl)carbamoyl)morpholine-4-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((2-(2,6-dioxopiperidin-3-yl)-1-oxoisoindolin-5-yl)carbamoyl)piperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-((2-(2-((2-(2,6-dioxopiperidin-3-yl)-1-oxoisoindolin-5-yl)amino)-2-oxoethoxy)ethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-((2-(2,6-dioxopiperidin-3-yl)-1-oxoisoindolin-5-yl)carbamoyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-((2-(2,6-dioxopiperidin-3-yl)-1-oxoisoindolin-5-yl)amino)-2-oxoethyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl((1R,3R)-3-(7-(4-(4-(2-(2,6-dioxopiperidin-3-yl)-1,3-dioxoisoindolin-5-yl)piperazine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate; and
methyl((1R,3R)-3-(7-(4-(4-((4-(2-(2,6-dioxopiperidin-3-yl)-3-oxoisoindolin-4-yl)piperazin-1-yl)methyl)piperidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
or a pharmaceutically acceptable salt thereof.

42. The compound of claim 1, or a pharmaceutically acceptable salt thereof, which is deuterated.

43. A pharmaceutical composition, comprising a compound of claim 1, or a pharmaceutically acceptable salt thereof, and a pharmaceutically acceptable carrier.

44. A method of inhibiting an activity of the V617F variant of JAK2 kinase, comprising contacting the kinase with a compound of claim 1, or a pharmaceutically acceptable salt thereof.

45. A method of treating cancer in a patient in need thereof, the method comprising administering to the patient a therapeutically effective amount of a compound of claim 1, or a pharmaceutically acceptable salt thereof.

46. The method of claim 45, wherein the cancer is selected from bladder cancer, breast cancer, cervical cancer, colorectal cancer, cancer of the small intestine, colon cancer, rectal cancer, cancer of the anus, endometrial cancer, gastric cancer, head and neck cancer, kidney cancer, liver cancer, lung cancer, ovarian cancer, prostate cancer, testicular cancer, uterine cancer, vulvar cancer, esophageal cancer, gall bladder cancer, pancreatic cancer, stomach cancer, thyroid cancer, parathyroid cancer, neuroendocrine cancer, skin cancer, and brain cancer.

47. The method of claim 45, wherein the cancer is a hematological cancer.

48. The method of claim 45, wherein the cancer is selected from leukemia,
lymphoma, multiple myeloma, chronic lymphocytic lymphoma, adult T cell leukemia, acute myeloid leukemia, B-cell lymphoma, cutaneous T-cell lymphoma, acute myelogenous leukemia, Hodgkin's or non-Hodgkin's lymphoma, a myeloproliferative neoplasm), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphoma, and Burkitt's lymphoma.

49. A method of treating a myeloproliferative disorder in a patient in need thereof, the method comprising administering to the patient a therapeutically effective amount of a compound of claim 1, or a pharmaceutically acceptable salt thereof.

50. The method of claim 49, wherein the myeloproliferative disorder is selected from polycythemia vera, essential thrombocythemia, myelofibrosis with myeloid metaplasia, primary myelofibrosis, post-essential thrombocythemia myelofibrosis, post polycythemia vera myelofibrosis, chronic myelogenous leukemia, chronic myelomonocytic leukemia, hypereosinophilic syndrome, and systemic mast cell disease.

51. The method of claim 49, wherein the myeloproliferative disorder is selected from polycythemia vera, essential thrombocythemia, myelofibrosis with myeloid metaplasia, primary myelofibrosis, post-essential thrombocythemia myelofibrosis, and post polycythemia vera myelofibrosis.

52. A method of treating myelodysplastic syndrome in a patient in need thereof, the method comprising administering to the patient a therapeutically effective amount of a compound of claim 1, or a pharmaceutically acceptable salt thereof.
